(19) United States
(12) Patent Application Publication
(10) Pub. No.: US 2024/0316059 A1
(43) Pub. Date: Sep. 26, 2024

(54) PEPTIDE DRUG CONJUGATES

(71) Applicant: Oncopeptides AB, Stockholm (SE)

(72) Inventors: Antonio BERMEJO GOMEZ, Stockholm (SE); Praveen KUMAR CHINTHAKINDI, Stockholm (SE); Charles DEMMER, Stockholm (SE); Katarina FARNEGARDH, Stockholm (SE); Mathias FARNEGARDH, Stockholm (SE); Fredrik LEHMANN, Stockholm (SE); Ellen SANTANGELO, Stockholm (SE)

(73) Assignee: Oncopeptides AB, Stockholm (SE)

(21) Appl. No.: 18/570,814

(22) PCT Filed: Jun. 20, 2022
(86) PCT No.: PCT/EP2022/066756

§ 371 Date: Dec. 15, 2023

(30) Foreign Application Priority Data
Jun. 18, 2021 (GB) 2108818.2

(51) Int. Cl. A61K 31/5377; A61K 31/4184; A61K 31/437; A61K 45/06; A61P 35/00; C07D 235/16; C07D 471/04

(52) U.S. Cl. CPC A61K 31/5377; A61K 31/4184; A61K 31/437; A61K 45/06; A61P 35/00; C07D 235/16; C07D 471/04

(57) ABSTRACT

The invention provides compounds of formula (I), that are peptidase enhanced cytotoxics. The invention further provides pharmaceutical compositions comprising the compounds, and the use of the compounds or pharmaceutical compositions as a medicament, particularly for use in the treatment or prophylaxis of cancers such as multiple myeloma, osteosarcoma, breast cancer, lung cancer, ovarian cancer, leukaemia and lymphoma.

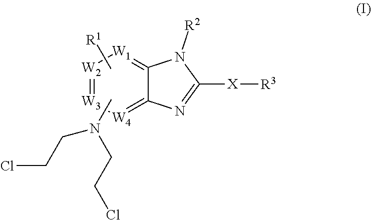

### FIELD OF THE INVENTION

The present invention relates to compounds that are peptide drug conjugates (PDCs), and the use of such compounds in the treatment of diseases such as cancer.

### BACKGROUND OF THE INVENTION

An increased expression of various hydrolytic enzymes like peptidases, esterases, and proteases has been described in several types of human malignancies, especially those characterized by fast-growing and aggressive phenotypes (Pharmacol Ther. 1999; 83: 67-123). The Zn²⁺-dependent membrane-bound ectopeptidase aminopeptidase N (APN, also known as CD13), widely expressed in mammalian cells, plays an important role in the development of cancer, including processes like tumor cell invasion, differentiation, proliferation, apoptosis, motility, and angiogenesis (Cancer Lett. 2006; 243: 135-43; Curr Med Chem. 2007; 14: 639-47; Curr Med Chem. 2008; 15: 2850-65). The multiple functions of APN have led to its designation as a “moonlighting ectoenzyme” (Trends Mol Med. 2008; 14: 361-71). Together, these abilities suggest APN as a potential therapeutic target in the treatment of cancer. Different approaches have been used to develop new drugs directed at this target, including enzyme inhibitors and APN-targeted carrier constructs (Cancer Sci. 2011; 102: 501-8).

One class of drug that may find use in the treatment of cancers by exploitation of hydrolytic enzymes, such as peptidases, esterases, and proteases, which are present in malignancies, is peptide drug conjugates (PDCs). The present inventors have found a new family of peptide drug conjugate, which finds utility in the treatment of cancers.

### Summary of the Invention

The present invention provides a compound of formula (I), or a pharmaceutically acceptable salt, ester, amide or carbamate thereof, including a salt of such an ester, amide or carbamate, according to claim 1.

The present inventors have found that compounds of formula (I) are potent anticancer agents. In particular, the present inventors have demonstrated in an in vitro cytotoxicity assay that compounds of formula (I) display excellent in vitro cytotoxicity towards various haematological cancer cell lines. The present inventors have also found that compounds of formula (I) were effective at reducing tumor growth in an in ovo chicken embryo xenograft model of lymphoma. Furthermore, the present inventors have found that compounds of formula (I) are readily hydrolysed within cancer cells to form metabolites that are preferentially sequestered and retained within cells and have strong alkylating activity.

The invention further provides a pharmaceutical composition comprising a compound according to the invention, together with a pharmaceutically acceptable carrier. The pharmaceutical composition may optionally comprise one or more additional therapeutic agents, for example a steroid, checkpoint inhibitor, nuclear transport inhibitor, anti-apoptotic inhibitor, cell therapy (including adoptive cell therapy), bi-specific T-cell engager (BiTE), immunomodulatory imide drug (IMiD), proteasome inhibitor (PI), histone deacetylase (HDAC) inhibitor, peptide drug conjugate (PDC), an alkylator or DNA intercalator.

The invention further provides a compound or a pharmaceutical composition according to the invention for use as a medicament. Further, there is also provided a compound or a pharmaceutical composition according to the invention for use in the treatment or prophylaxis of cancer, for example multiple myeloma, osteosarcoma, breast cancer, lung cancer, ovarian cancer, leukaemia and lymphoma.

The invention further provides a method of treating a patient which comprises administering a pharmaceutically effective amount of a compound or pharmaceutical composition according to the invention.

The invention further provides the use of a compound according to the invention for the manufacture of a medicament for the treatment or prophylaxis of cancer.

The present invention further provides further compounds of the invention according to claim 25.

### BRIEF DESCRIPTION OF THE DRAWINGS

FIG. 1 shows the results of intracellular and extracellular concentration measurements of the metabolite compound (Example compound 18) of the invention (identified after treatment with Example compound 1 of the invention) (FIG. 1A), and the bendamustine (FIG. 1A) and melphalan (FIG. 1B) comparators following in vitro treatment of MM.1S cells.

FIG. 2 shows the effect of Example compound 1 on tumour growth on xenografts initiated from SU-DHL-4 human lymphoma cell line in in ovo chicken embryo model.

FIGS. 3(A) to (G) show the results of intracellular and extracellular concentration measurements of compounds of the invention and their metabolites following in vitro treatment of MM.1S cells.

FIGS. 4 (A) to (C) show the results of an assay assessing DNA damage caused by compounds of the invention and controls.

FIGS. 5 (A) and (B) show the results of an assay assessing DNA fragmentation caused by compounds of the invention and controls.

### DETAILED DESCRIPTION OF THE INVENTION

The present inventors have found a new class of PDC that are highly cytotoxic towards human cancer cells, and particularly human haematological cancer cells. As described in the Examples section, various example compounds of the invention have been synthesised and their cytotoxicity towards several haematological cancer cell lines was tested in an in vitro cytotoxicity assay. The present inventors found that the compounds of the present invention were highly potent and display selectivity towards haematological cancer cells, as shown by the lower cytotoxicity of the compounds towards the fibroblast cell line, BJ. The present inventors have also found that Example compound 1 is particularly effective at inhibiting tumor growth in an in ovo chicken embryo xenograft model using the human lymphoma cell line, SU-DHL-4.

In a further study, the present inventors found that example compounds of the invention were readily hydrolysed within MM.1S cancer cells to form metabolites that were sequestered and retained within the cells and had strong alkylating activity, thus demonstrating that the compounds of the present invention are an effective new class of PDC for the treatment or prophylaxis of cancer, and particularly the treatment or prophylaxis of haematological cancers.

The present invention provides compounds of formula (I):

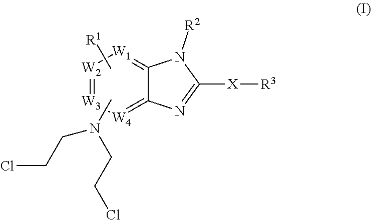

W₁, W₂, W₃ and W₄ are each CH, or one of W₁, W₂, W₃ and W₄ is N and the others are CH.

In an embodiment, W₂, W₃ and W₄ are each CH, and W₁ is N.

It is readily seen that the —N(CH₂CH₂Cl)₂ group is attached to the core part of the molecule through a carbon atom on the ring. For the relevant W group, the H in the CH group is correspondingly absent.

In preferred embodiments, W₁, W₂, W₃ and W₄ are each CH and so formula (I) has the formula (Ia):

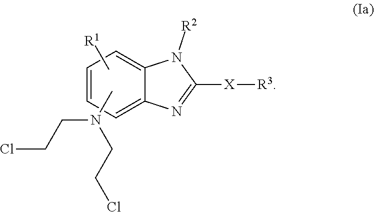

In preferred embodiments, formula (I) is according to formula (Ib),

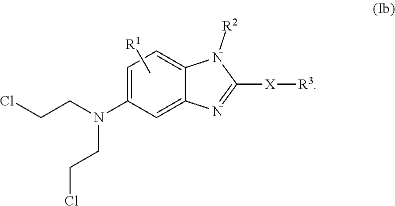

In the compounds of formula (I), X is C₁₋₆alkylene. For example, X may be C₁₋₄alkylene, C₁₋₃alkylene, C₁₋₂alkylene, C₂alkylene or C₁alkylene. X may be a linear or branched alkylene. The alkylene at position X forms a link between the imidazole moiety and the peptide portion of the compound of the invention (i.e. the R³ portion of the compound, as described below). Alkylene linkers at the X position that are one carbon or two carbons in length have been found to be particularly effective. As such, in preferred embodiments, X is a C₁alkylene (i.e. —CH₂—) or linear C₂alkylene (i.e. —CH₂—CH₂—).

In the compounds of formula (I), R¹ is selected from the group consisting of H; C₁₋₄alkyl, optionally substituted by 1, 2 or 3 groups independently selected from halogen; and halogen (for example selected from the group consisting of H; C₁₋₄alkyl; and halogen). For example, R¹ may be selected from the group consisting of H, methyl, ethyl, propyl, isopropyl, butyl, isobutyl, sec-butyl, tert-butyl, F, Cl, Br and I. Optionally, those groups may be substituted by 1, 2 or 3 groups independently selected from halogen. In certain embodiments, R¹ is selected from the group consisting of H, methyl, ethyl, propyl, isopropyl, butyl, isobutyl, sec-butyl, tert-butyl, F and Cl. In certain preferred embodiments, R¹ is H.

In the compounds of formula (I), R² is selected from the group consisting of H; phenyl optionally substituted by 1, 2 or 3 groups independently selected from halogen; and —C₁₋₆alkyl optionally substituted by 1, 2 or 3 groups independently selected from halogen. For example, R² may be selected from the group consisting of H; phenyl optionally substituted by 1, 2 or 3 F or Cl; and —C₁₋₄alkyl (for example, methyl, ethyl, propyl, isopropyl, butyl, isobutyl, sec-butyl or tert-butyl) optionally substituted by 1, 2 or 3 F or Cl. In an embodiment, R² is selected from the group consisting of phenyl optionally substituted by 1, 2 or 3 groups independently selected from halogen; and —C₁₋₆alkyl optionally substituted by 1, 2 or 3 groups independently selected from halogen, for example —C₁₋₄alkyl (for example methyl) optionally substituted by 1, 2 or 3 groups selected from halogen. In certain embodiments, R² is an unsubstituted —C₁₋₂alkyl (for example, methyl) or an unsubstituted phenyl.

In the compounds of formula (I), R³ is a group according to formula (II):

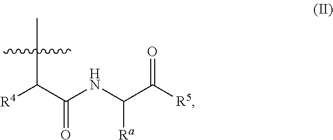

or formula (III):

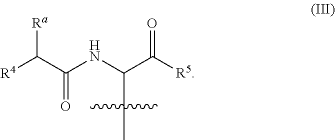

In preferred embodiments, R³ is a group according to formula (IIa):

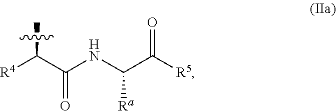

or formula (IIIa):

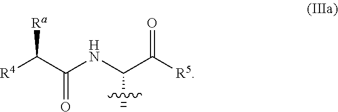

For the avoidance of doubt, [symbol] in formulas (II), (III), (IIa) and (IIIa) denotes the point of attachment of formula (II), (III), (IIa) or (IIIa) to formula (I), (Ia) or (IIa).

In formulas (II), (IIa), (III) and (IIIa), R⁴ is selected from the group consisting of N(R^{c})(R^{d}) and formula (IV):

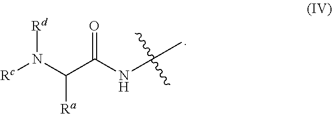

In preferred embodiments, R⁴ is selected from the group consisting of N(R^{c})(R^{d}) and formula (IVa):

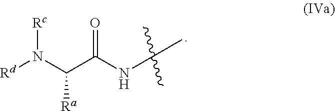

For the avoidance of doubt, [symbol] in formula (IV) and (IVa) denotes the point of attachment of formula (IV) or (IVa) to formula (II), (III), (IIa) or (IIIa).

In compounds of formula (I), when R⁴ is formula (IV) or (IVa), R⁵ is R^{b}.

R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀ aryl, or a 3 to 12 membered heterocycle comprising one or more O, N or S atoms and optionally substituted by 1, 2 or 3 halogens, and/or wherein said alkyl is optionally interrupted by 1, 2 or 3 O, N or S atoms.

For example, when R⁴ is formula (IV) or (IVa), R⁵ (which is R^{b}) may be selected from the group consisting of —OH and —OC₁₋₄alkyl (for example, methoxy, ethoxy, propoxy, isopropoxy, butoxy, isobutoxy, sec-butoxy, or tert-butoxy). Preferably, when R⁴ is formula (IV) or (IVa), R⁵ is selected from the group consisting of —OH, and —OC₁₋₃alkyl (for example, methoxy, ethoxy, propoxy, or isopropoxy). More preferably, when R⁴ is formula (IV) or (IVa), R⁵ is selected from the group consisting of methoxy, ethoxy or isopropoxy.

In compounds of formula (Ia), when R⁴ is N(R^{c})(R^{d}), R⁵ is selected from the group consisting of R^{b} and formula (V):

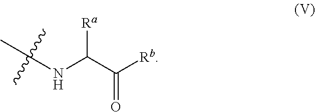

In preferred embodiments, when R⁴ is N(R^{c})(R^{d}), R⁵ is —OH, —OC₁₋₆alkyl or formula (Va):

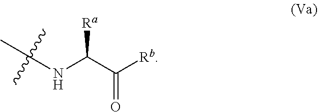

For the avoidance of doubt, [symbol] in formula (V) and (Va) denotes the point of attachment of formula (V) or (Va) to formula (II), (III), (IIa) or (IIIa).

In embodiments wherein R⁴ is N(R^{c})(R^{d}), R⁵ (being R^{b}) may be selected from the group consisting of —OH, —OC₁₋₄alkyl (for example, methoxy, ethoxy, propoxy, isopropoxy, butoxy, isobutoxy, sec-butoxy or tert-butoxy), or R⁵ may be selected from formula (V) and formula (Va). Preferably, when R⁴ is N(R^{c})(R^{d}), R⁵ is selected from the group consisting of —OH, —OC₁₋₃alkyl (for example, methoxy, ethoxy, propoxy or isopropoxy) and formula (Va). More preferably, when R⁴ is N(R^{c})(R^{d}), R⁵ is selected from the group consisting of methoxy, ethoxy or isopropoxy.

The present inventors have found that compounds of formula (I), in which R⁴ is N(R^{c})(R^{d}), formula (IV) or (IVa), and R⁵ (being R^{b}) is —OC₁₋₃alkyl (for example, methoxy, ethoxy or isopropoxy), are especially cytotoxic towards cancer cells. Thus, in certain preferred embodiments, in the compounds of formula (I), R⁵ is —OC₁₋₃alkyl (for example, methoxy, ethoxy or isopropoxy). That is to say that, when R⁴ is N(R^{c})(R^{d}), formula (IV) or (IVa), R⁵ is preferably —OC₁₋₃alkyl (for example, methoxy, ethoxy or isopropoxy).

In certain embodiments, R⁵ is —OH.

In compounds of formula (I), each R^{a} is independently selected from the group consisting of H; C₁₋₆alkyl; —CH₂-phenyl; or —CH₂-3 to 12-membered heterocyclyl comprising 1, 2, 3 or 4 heteroatoms selected from N, O and S; wherein said C₁₋₆alkyl is optionally substituted by 1, 2 or 3 substituents independently selected from the group consisting of —OH, —OC₁₋₆alkyl, —NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂. In certain embodiments, each R^{a} is independently selected from the group consisting of H; —C₁₋₆alkyl; —CH₂-indolyl; —CH₂-phenyl; and —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen (for example, F, Cl, Br or 1); and phenyl is optionally substituted by 1, 2 or 3 substituents independently selected from the group consisting of halogen (for example, F, Cl, Br or 1); —NH₂; —OH; —OC₁₋₆alkyl; and —NO₂. Preferably, each R^{a} is independently selected from the group consisting of H; C₁₋₄alkyl; and —CH₂-phenyl; wherein alkyl or phenyl is optionally substituted by 1 or 2 halogen (for example, F or Cl). For example, each R^{a} may independently be methyl, ethyl, propyl, isopropyl, butyl, isobutyl, sec-butyl, —CH₂-phenyl, —CH₂-fluorophenyl, —CH₂-chlorophenyl, —CH₂— difluorophenyl, or —CH₂-dichlorophenyl. In certain preferred embodiments, each R^{a} may independently be isopropyl, isobutyl, sec-butyl, —CH₂-phenyl, or —CH₂— fluorophenyl (i.e. 2-fluorobenzyl, 3-fluorobenzyl or 4-fluorobenzyl).

In compounds of formula (I), R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or a 3 to 12 membered heterocycle comprising one or more O, N or S atoms and optionally substituted by 1, 2 or 3 halogens, and/or wherein said alkyl is optionally interrupted by 1, 2 or 3 O, N or S atoms. In certain embodiments, R^{b} is —OH or —OC₁₋₆alkyl, wherein said alkyl is optionally interrupted by 1, 2 or 3 O or N atoms. In certain further embodiments, R^{b} is —OH or —OC₁₋₆alkyl, for example —OC₁₋₆alkyl. Preferably, R^{b} is —OC₁₋₄alkyl. For example, R^{b} may be methoxy, ethoxy, propoxy, isopropoxy, butoxy, isobutoxy or sec-butoxy. In certain preferred embodiments, R^{b} is methoxy, ethoxy or isopropoxy. More preferably, R^{b} is ethoxy.

In certain embodiments, R^{b} is —OH.

R^{c} and R^{d} are each independently selected from the group consisting of H, —C₁₋₆alkyl, —C(O)C₁₋₆alkyl, and —CH₂-phenyl, wherein said alkyl or said phenyl is optionally substituted by 1, 2 or 3 groups selected from halogen. In preferred compounds, R^{c} is H and R^{d} is selected from H, —C₁₋₆alkyl and —C(O)C₁₋₆alkyl, wherein said alkyl is optionally substituted by 1, 2 or 3 groups selected from halogen. For example, R^{c} is H and R^{d} is selected from H, —C₁₋₄alkyl and C(O)C₁₋₄alkyl. For example, R^{c} is H and R^{d} is selected from H, methyl, ethyl, propyl, isopropyl, butyl, isobutyl, sec-butyl, —C(O)methyl, —C(O)ethyl, —C(O)propyl, —C(O)isopropyl, —C(O)butyl, —C(O)isobutyl and —C(O)sec-butyl. Preferably, R^{c} is H and R^{d} is selected from H, methyl and —C(O)methyl.

Preferably, R^{c} is H and R^{d} is H.

In compounds of formula (I), R^{e} and R^{f} are each independently selected from the group consisting of H and —C₁₋₆alkyl, wherein said alkyl is optionally substituted by 1, 2 or 3 groups selected from halogen; or R^{e} and R^{f} together with the nitrogen atom to which they are attached form a 4-, 5- or 6-membered heterocycle which is optionally substituted by 1, 2 or 3 groups selected from halogen. In preferred compounds, R^{e} and R^{f} are each independently selected from the group consisting of H, methyl, ethyl, propyl, isopropyl, butyl, isobutyl and sec-butyl. For example, R^{e} and R^{f} are each independently selected from the group consisting of H and methyl. Preferably, R^{e} and R^{f} are both H, both methyl, or one of R^{e} and R^{f} is H and the other is methyl.

In certain embodiments, the compounds of the invention are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIa) or (IIIa);
R⁴ is selected from the group consisting of N(R^{c})(R^{d}) and formula (IVa);
when R⁴ is formula (IVa), R⁵ (being R^{b}) is —OC₁₋₄alkyl; and when R⁴ is N(R^{c})(R^{d}), R⁵ is —OC₁₋₄alkyl or formula (Va);
each R^{a} is independently selected from the group consisting of H; —C₁₋₆alkyl;
—CH₂-indolyl; —CH₂-phenyl; or —CH₂-5-membered heteroaryl comprising 1, 2, 3 or 4 N or S atoms; wherein said —C₁₋₆alkyl is optionally substituted by —OH; —NH₂; —NHC(═NH)NH₂; —C(O)OH; —C(O)NH₂; —SH; —SCH₃; or halogen; and said phenyl is optionally substituted by 1, 2 or 3 substituents independently selected from the group consisting of halogen; —NH₂; —OH; —O—C₁₋₆alkyl; and —NO₂;
R^{b} is —OC₁₋₆alkyl; and
R^{c} is H and R^{d} is selected from H, —C₁₋₄alkyl and —C(O)C₁₋₄alkyl;
or a pharmaceutically acceptable salt, ester, amide or carbamate thereof, including a salt of such an ester, amide or carbamate.

In certain other embodiments, the compound of the present invention is according to formula (Ia), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is N(R^{c})(R^{d}) or formula (IVa);
R⁵ (being R^{b}) is —OC₁₋₃alkyl; and
each R^{a} is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —OC₁₋₆alkyl; and —NO₂;
or a pharmaceutically acceptable salt, ester, amide or carbamate thereof, including a salt of such an ester, amide or carbamate.

The present inventors have found that compounds of present invention are especially cytotoxic towards haematological cancer cells in an in vitro cytotoxicity assay and in ovo chicken embryo xenograft model of lymphoma, when the compound is according to formula (Ia), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is N(R^{c})(R^{d}) or formula (IVa);
R⁵ (being R^{b}) is —OC₁₋₃alkyl; and
each R¹ is independently selected from the group consisting of isopropyl, isobutyl and —CH₂-phenyl optionally substituted by 1 F (for example, 2-fluorobenzyl, 3-fluorobenzyl or 4-fluorobenzyl);
or a pharmaceutically acceptable salt, ester, amide or carbamate thereof, including a salt of such an ester, amide or carbamate.

In certain other embodiments, the compounds of the invention are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIa);
R⁴ is NH₂;
R⁵ (being R^{b}) is —OC₁₋₄alkyl; and
each R¹ is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain other embodiments, the compounds of the invention are according to formula (Ia), wherein,
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIIa);
R⁴ is NH₂;
R⁵ is —OC₁₋₄alkyl; and
each R^{a} is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain other embodiments, the compounds of the invention are according to formula (Ia), wherein
X is —C₁₋₂alkylene (i.e. —CH₂— or —CH₂—CH₂—);
R¹ is H;
R² is —C₁₋₂alkyl optionally substituted by 1, 2 or 3 F, or phenyl optionally substituted by 1, 2 or 3 F, for example, R² may be an unsubstituted —C₁₋₂alkyl or an unsubstituted phenyl;
R³ is formula (IIIa);
R⁴ is formula (IVa);
R⁵ is —OC₁₋₄alkyl; and
each R^{a} is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein —C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂;
or a pharmaceutically acceptable salt, ester, amide or carbamate thereof, including a salt of such an ester, amide or carbamate.

In certain preferred embodiments, the compound of the invention is:
Ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1);
Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate (Example compound 2);
Ethyl (2S)-2-[[(2S)-2-amino-4-[5-[bis(2-chloroethyl)amino]-1-methyl-benzimidazol-2-yl]butanoyl]amino]-3-methyl-butanoate (Example compound 3);
Ethyl (2S)-2-[[(2S)-2-amino-3-[5-[bis(2-chloroethyl)amino]-1-methyl-benzimidazol-2-yl]propanoyl]amino]-4-methyl-pentanoate (Example compound 4);
Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate (Example compound 5);
Ethyl (2S)-2-[[(2S)-2-amino-3-[5-[bis(2-chloroethyl)amino]-1-methyl-benzimidazol-2-yl]propanoyl]amino]-3-methyl-butanoate (Example compound 6);
Ethyl (2S)-2-[[(2S)-2-amino-4-methyl-pentanoyl]amino]-4-[5-[bis(2-chloroethyl)amino]-1-methyl-benzimidazol-2-yl]butanoate (Example compound 7);
Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate (Example compound 8);
Ethyl (2S)-2-[[(2S)-2-amino-3-methyl-butanoyl]amino]-4-[5-[bis(2-chloroethyl)amino]-1-methyl-benzimidazol-2-yl]butanoate (Example compound 9);
Ethyl (2S)-2-[[(2S)-2-amino-4-methyl-pentanoyl]amino]-3-[5-[bis(2-chloroethyl)amino]-1-methyl-benzimidazol-2-yl]propanoate (Example compound 10);
Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate (Example compound 11);
Ethyl (2S)-2-[[(2S)-2-amino-3-methyl-butanoyl]amino]-3-[5-[bis(2-chloroethyl)amino]-1-methyl-benzimidazol-2-yl]propanoate (Example compound 12);
Ethyl (2S)-2-[[(2S)-2-amino-4-[5-[bis(2-chloroethyl)amino]-1-phenyl-benzimidazol-2-yl]butanoyl]amino]-4-methyl-pentanoate (Example compound 13);
Methyl (2S)-2-[[(2S)-2-amino-3-[5-[bis(2-chloroethyl)amino]-1-methyl-benzimidazol-2-yl]propanoyl]amino]-4-methyl-pentanoate (Example compound 14);
Isopropyl (2S)-2-[[(2S)-2-amino-3-[5-[bis(2-chloroethyl)amino]-1-methyl-benzimidazol-2-yl]propanoyl]amino]-4-methyl-pentanoate (Example compound 15);
Ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 16);
Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 21)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate (Example compound 22)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate (Example compound 23)
2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate (Example compound 24) 2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate (Example compound 25)
Isopropyl (2S)-2-[[(2S)-2-amino-4-[5-[bis(2-chloroethyl)amino]-1-methyl-benzimidazol-2-yl]butanoyl]amino]-4-methyl-pentanoate (Example compound 26)
Methyl (2S)-2-[[(2S)-2-amino-4-[5-[bis(2-chloroethyl)amino]-1-methyl-benzimidazol-2-yl]butanoyl]amino]-4-methyl-pentanoate (Example compound 27)
3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate (Example compound 28)
(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate (Example compound 29)
(2S)-2-[[(2S)-2-Amino-4-[5-[bis(2-chloroethyl)amino]-1-methyl-benzimidazol-2-yl]butanoyl]amino]-N,N,4-trimethyl-pentanamide (Example compound 30)
(2S)-2-[[(2S)-2-amino-4-[5-[bis(2-chloroethyl)amino]-1-methyl-benzimidazol-2-yl]butanoyl]amino]-N,4-dimethyl-pentanamide (Example compound 31)
Ethyl (2S)-2-[[(2S)-4-[5-[bis(2-chloroethyl)amino]-1-methyl-benzimidazol-2-yl]-2-(methylamino)butanoyl]amino]-4-methyl-pentanoate (Example compound 32)
Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate (Example compound 34)
Ethyl (2S)-2-[[(2S)-2-acetamido-3-[5-[bis(2-chloroethyl)amino]-1-ethyl-benzimidazol-2-yl]propanoyl]amino]-4methyl-pentanoate (Example compound 35)
Ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate (Example compound 36)
Ethyl (2S)-2-amino-4-[5-[bis(2-chloroethyl)amino]-1-methyl-benzimidazol-2-yl]butanoate (Example compound 38)
Ethyl (2R)-2-[[(2S)-2-amino-4-[5-[bis(2-chloroethyl)amino]-1-methyl-benzimidazol-2-yl]butanoyl]amino]-4-methyl-pentanoate (Example compound 45)
Ethyl (2S)-2-[[(2R)-2-amino-4-[5-[bis(2-chloroethyl)amino]-1-methyl-benzimidazol-2-yl]butanoyl]amino]-4-methyl-pentanoate (Example compound 46)
Ethyl (2R)-2-[[(2)-2-amino-4-[5-[bis(2-chloroethyl)amino]-1-methyl-benzimidazol-2-yl]butanoyl]amino]-4-methyl-pentanoate (Example compound 47)
Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate (Example compound 48); and
Ethyl (2S)-2-[[(2S)-2-amino-4-[5-[bis(2-chloro-1,1,2,2-tetraeuterio-ethyl)amino]-1-methyl-benzimidazol-2-yl]butanoyl]amino]-4-methyl-pentanoate (Example compound 50)
or a pharmaceutically acceptable salt, amide or carbamate thereof, including a salt of such a amide or carbamate.

The compounds of the present invention may be prepared using methods known to those skilled in the art of organic chemistry. Exemplary procedures for the preparation of compounds of formula (I) are described in the Examples section.

In embodiments, the compound of the invention may comprise an isotope atom. As defined herein, an isotope atom is an atom of an element that is not the most common naturally occurring isotope. Deuterium is a safe and stable isotope of hydrogen. In one embodiment, the compound of the invention has a deuterium abundance level greater than the naturally occurring abundance of deuterium. The naturally occurring abundance of deuterium is 0.0156 mol %, wherein mol % is the percentage of the total moles of a sample's hydrogen that is deuterium. Therefore, in 1 mole of naturally occurring hydrogen 0.156 mmol is deuterium, or in a sample of 6.022×10²³ naturally occurring hydrogen atoms there are 9.39×10¹⁹ atoms of deuterium, or in a sample of 6413 naturally occurring hydrogen atoms there is one atom of deuterium. A deuterium abundance level greater than the naturally occurring abundance of deuterium may be at least 1 mol %, 5 mol %, 10 mol %, 50 mol %, 90 mol % or 98 mol % deuterium. In certain embodiments, the compound of the invention has a deuterium abundance level of at least 1 mol %, 5 mol %, 10 mol %, 50 mol %, 90 mol % or 98 mol % deuterium. Procedures for preparing deuterated compounds are known in the art. See for example Sajiki, New Horizons of Process Chemistry (2017), Springer, pg 29-40, and Hanson, The Organic Chemistry of Isotopic Labelling (2011), Chapter 3, RSC Publishing.

Depending upon the substituents present in compounds of the invention, the compounds may form esters, amides, carbamates and/or salts. Salts of compounds of the invention which are suitable for use in medicine are those wherein a counterion is pharmaceutically acceptable. However, salts having non-pharmaceutically acceptable counterions are within the scope of the present invention, for example, for use as intermediates in the preparation of the compounds of the invention and their pharmaceutically acceptable salts, and physiologically functional derivatives. The term “physiologically functional derivative” refers to a chemical derivative of a compound of the invention that has the same physiological function as the compound of the invention, for example, by being convertible in the body thereto. Esters, amides and carbamates are examples of physiologically functional derivatives.

Suitable salts forms suitable for use in the present invention include those formed with organic or inorganic acids or bases. In particular, suitable salts formed with acids according to the invention include those formed with mineral acids, strong organic carboxylic acids, such as alkanecarboxylic acids of 1 to 4 carbon atoms which are unsubstituted or substituted, for example, by halogen, or such as saturated or unsaturated dicarboxylic acids, or such as hydroxycarboxylic acids, or such as amino acids, or with organic sulfonic acids, such as (C₁-C₄)-alkyl- or aryl-sulfonic acids which are unsubstituted or substituted, for example by halogen. Pharmaceutically acceptable acid addition salts include those formed from hydrochloric, hydrobromic, sulphuric, nitric, citric, tartaric, acetic, phosphoric, lactic, pyruvic, acetic, trifluoroacetic, succinic, perchloric, fumaric, maleic, glycolic, lactic, salicylic, oxaloacetic, methanesulfonic, ethanesulfonic, p-toluenesulfonic, formic, benzoic, malonic, naphthalene-2-sulfonic, benzenesulfonic, isethionic, ascorbic, malic, phthalic, aspartic, and glutamic acids, lysine and arginine. Other acids such as oxalic, while not in themselves pharmaceutically acceptable, may be useful as intermediates in obtaining the compounds of the invention and their pharmaceutical acceptable acid addition salts.

Pharmaceutically acceptable base salts include ammonium salts, alkali metal salts, for example those of potassium and sodium, alkaline earth metal salts, for example those of calcium and magnesium, and salts with organic bases, for example dicyclohexylamine, N-methyl-D-glucomine, morpholine, thiomorpholine, piperidine, pyrrolidine, a mono-, di- or tri-lower alkylamine, for example ethyl-, tert-butyl-, diethyl-, diisopropyl-, triethyl-, tributyl- or dimethyl-propylamine, or a mono-, di- or trihydroxy-lower alkylamine, for example mono-, di- or triethanolamine. Corresponding internal salts may furthermore be formed.

Preferred salts of a compound of the present invention include acid addition salts such as those formed from hydrochloric, hydrobromic, acetic, p-toluenesulfonic, tartaric, sulphuric, succinic, phosphoric, oxalic, nitric, methanesulfonic, malic, maleic and citric acid. More preferably, the salt of a compound of the present invention is the hydrochloride salt (i.e. the addition salt formed from hydrochloric acid).

A compound which is itself inactive, but which, upon administration to the recipient, is capable of being converted into an active drug compound is known as a “prodrug”.

A prodrug may, for example, be converted within the body, e.g. by hydrolysis in the blood, into an active form that has medical effects. Pharmaceutically acceptable prodrugs are described in T. Higuchi and V. Stella, Prodrugs as Novel Delivery Systems, Vol. 14 of the A. C. S. Symposium Series (1976); “Design of Prodrugs” ed. H. Bundgaard, Elsevier, 1985; and in Edward B. Roche, ed., Bioreversible Carriers in Drug Design, American Pharmaceutical Association and Pergamon Press, 1987.

The compounds of claim 1 or claim 25 of the present invention (and their corresponding dependent claims) can be provided in the form of a prodrug. Examples of prodrugs include esters, amides and carbamates.

Compounds of the invention may have an appropriate group converted to an ester, an amide or a carbamate. Thus typical ester and amide groups formed from an acid group in a compound of the invention include —COOR^{G}, —CONR^{G}₂, —SO₂OR^{G}, or —SO₂N(R^{G})₂, while typical ester and amide and carbamate groups formed from an —OH or —NHR^{G} group in the compound of the invention include —OC(O)R^{G}, —NR^{G}C(O)R^{G}, —NR^{G}CO₂R^{G}, —OSO₂R^{G}, and —NR^{G}SO₂R^{G}, where R^{G} is selected from the group consisting of C₁₋₈alkyl, C₂₋₈alkenyl, C₂₋₈alkynyl, C₃₋₈cycloalkyl and C₃₋₈ cycloalkylC₁₋₈alkyl, haloC₁₋₈alkyl, dihaloC₁₋₈alkyl, trihaloC₁₋₈alkyl, phenyl and phenylC₁₋₄alkyl; more preferably R^{G} is selected from the group consisting of C₁₋₆alkyl, C₂₋₆alkenyl, C₂₋₆alkynyl, C₃₋₈cycloalkyl and C₃₋₈cycloalkylC₁₋₆alkyl.

Those skilled in the art of organic chemistry will appreciate that many organic compounds can form complexes with solvents in which they are reacted or from which they are precipitated or crystallized. These complexes are known as “solvates”. For example, a complex with water is known as a “hydrate”. The complex may incorporate a solvent in stoichiometric or non-stoichiometric amounts. Solvates are described in Water-Insoluble Drug Formulation, 2^{nd} edn, R. Lui, C R^{c} Press, page 553, and Byrn et al., Pharm. Res., 12(7), 1995, 945-954. Before it is made up in solution, a compound of the present invention, as well as esters, amides, carbamates and/or salts thereof, may be in the form of a solvate. Solvates of a compound of the present invention that are suitable for use as a medicament are those wherein the associated solvent is pharmaceutically acceptable. For example a hydrate is pharmaceutically acceptable solvate.

The present inventors have found that compounds of the present invention in which R⁵ is selected from the group consisting of —OC₁₋₆alkyl formula (V)

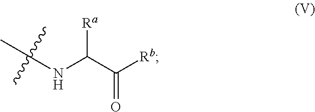

and formula (Va)

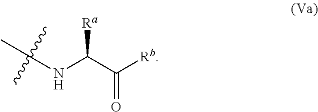

and R^{b} is a group selected from —N(R^{e})(R^{f}) and —OC₁₋₆alkyl, are readily hydrolysed within cancer cells to form metabolites that are preferentially sequestered and retained within cells and have strong alkylating activity.

Thus, the present invention also provides a metabolite, wherein said metabolite has a structure according to formula (I), (Ia) or (Ib), wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen;
R³ is a group according to formula (II), (Ill), (IIa) or (IIIa);
R⁴ is selected from the group consisting of N(R^{c})(R^{d}), formula (IV) or (IVa); when R⁴ is formula (IV) or (IVa), R⁵ is —OH; and when R⁴ is N(R^{c})(R^{d}), R⁵ is —OH, formula (V) or (Va);
each R^{a} is independently selected from the group consisting of H; —C₁₋₆alkyl; —CH₂— phenyl; or —CH₂-3 to 12-membered heterocyclyl comprising 1, 2, 3 or 4 heteroatoms selected from N, O and S; wherein said —C₁₋₆alkyl is optionally substituted by 1, 2 or 3 substituents independently selected from the group consisting of —OH, —OC₁₋₆alkyl, —NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂ and
R^{b} is OH;
or a salt thereof.

Preferred radicals within the metabolite compounds as described immediately above are as described hereinabove regarding compounds of Formula (I), (Ia) and (Ib). For example, in certain embodiments, the metabolite has a structure according to formula (I), (Ia) or (Ib), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is selected from the group consisting of N(R^{c})(R^{d}) or formula (IVa);
when R⁴ is formula (IVa), R⁵ is —OH; and when R⁴ is N(R^{c})(R^{d}), R⁵ is —OH or formula (Va);
each R^{a} is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein —C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —O—C₁₋₆alkyl; and —NO₂; and
R^{b} is OH;
or a salt thereof.

In certain embodiments, the metabolite has a structure according to formula (I), (Ia) or (Ib), wherein,
X is —C₁₋₂alkylene (i.e. —CH₂— or —CH₂—CH₂—);
R¹ is H;
R² is methyl or phenyl;
R³ is formula (IIa) or (IIIa);
R⁴ is NH₂ or formula (IVa);
R⁵ is —OH;
each R^{a} is independently selected from the group consisting of H; —C₁₋₆alkyl; —CH₂— indolyl; —CH₂-phenyl; or —CH₂-5-membered heteroaryl comprising 1, 2, 3 or 4 N or S atoms; wherein C₁₋₆alkyl is optionally substituted by —OH; —NH₂; —NHC(═NH)NH₂; —C(O)OH; —C(O)NH₂; —SH; —SCH₃; or halogen; and phenyl is optionally substituted by 1, 2 or 3 substituents independently selected from the group consisting of halogen; —NH₂; —OH; —OC₁₋₆alkyl; and —NO₂. Preferably, each R^{a} is independently selected from the group consisting of isopropyl, isobutyl and —CH₂-phenyl optionally substituted by 1 F (for example, 2-fluorobenzyl, 3-fluorobenzyl or 4-fluorobenzyl); or a salt thereof.

In certain preferred embodiments, the metabolite is:
(2S)-2-[[(2S)-2-amino-4-[5-[bis(2-chloroethyl)amino]-1-methyl-benzimidazol-2-yl]butanoyl]amino]-3-methyl-butanoic acid (Example compound 19); and
(2S)-2-[[(2S)-2-amino-4-[5-[bis(2-chloroethyl)amino]-1-methyl-benzimidazol-2-yl]butanoyl]amino]-4-methyl-pentanoic acid (Example compound 20);
(2S)-2-[[(2S)-4-[5-[bis(2-chloroethyl)amino]-1-methyl-benzimidazol-2-yl]-2-(methylamino)butanoyl]amino]-4-methyl-pentanoic acid (Example compound 33)
(2S)-2-[[(2S)-2-Amino-4-methyl-pentanoyl]amino]-4-[5-[bis(2-chloroethyl)amino]-1-methyl-benzimidazol-2-yl]butanoic acid (Example compound 37)
(2S)-2-[[(2S)-2-Amino-4-[5-[bis(2-chloroethyl)amino]-1-methyl-benzimidazol-2-yl]butanoyl]amino]-3-(4-fluorophenyl)propanoic acid (Example compound 39)
(2S)-2-[[(2S)-2-Amino-4-[5-[bis(2-chloroethyl)amino]-1-phenyl-benzimidazol-2-yl]butanoyl]amino]-4-methyl-pentanoic acid (Example compound 40) 2S)-2-[[(2S)-2-amino-3-[5-[bis(2-chloroethyl)amino]-1-methyl-benzimidazol-2-yl]propanoyl]amino]-3-methyl-butanoic acid (Example compound 42)
(2S)-2-[[(2S)-2-amino-4-methyl-pentanoyl]amino]-3-[5-[bis(2-chloroethyl)amino]-1-methyl-benzimidazol-2-yl]propanoic acid (Example compound 43)
(2S)-2-[[(2S)-2-amino-3-[5-[bis(2-chloroethyl)amino]-1-methyl-benzimidazol-2-yl]propanoyl]amino]-4-methyl-pentanoic acid (Example compound 44); and
(2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoic acid (Example compound 51),
or a salt thereof.

The present inventors have further found that compounds of the present invention are readily further hydrolysed within cancer cells to form further, more advanced metabolites that are preferentially sequestered and retained within cells and have strong alkylating activity. Thus, the present invention also provides a metabolite that has a structure according to formula (I), (Ia) or (Ib), wherein,
X is C₁₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl, optionally substituted by 1, 2 or 3 groups independently selected from halogen; and halogen (for example, F or Cl);
R² is selected from the group consisting of H; phenyl optionally substituted by 1, 2 or 3 groups independently selected from halogen (for example, F or Cl); and —C₁₋₆alkyl optionally substituted by 1, 2 or 3 groups independently selected from halogen (for example, F or Cl); and
R³ is formula (VIa)

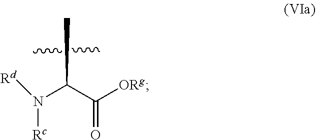

wherein, R^{c} and R^{d} are each independently selected from the group consisting of H, —C₁₋₆alkyl, —C(O)C₁₋₆alkyl and —CH₂-phenyl, wherein said alkyl or said phenyl is optionally substituted by 1, 2 or 3 groups selected from halogen (preferably, R^{c} and R^{d} are both H); and
R^{g} is selected from the group consisting of H and —C₁₋₆alkyl, wherein said alkyl is optionally substituted by 1, 2 or 3 groups selected from halogen (preferably R^{g} is H).

For the avoidance of doubt, [symbol] in formulas (VI) and (VIa) denotes the point of attachment of formula (VI) or (VIa) to formula (I), (Ia) or (Ib).

In certain embodiments, the metabolite has a structure according to formula (I), (Ia) or (Ib), wherein,
X is C₁₋₆alkylene;
R¹ is selected from the group consisting of H; —C₁₋₄alkyl; and halogen (for example, F or Cl);
R² is selected from the group consisting of H; phenyl substituted by 1, 2 or 3 halogens (for example, F or Cl); and —C₁₋₆alkyl substituted by 1, 2 or 3 halogens (for example, F or Cl) (for example selected from the group consisting of phenyl substituted by 1, 2 or 3 halogens (for example, F or Cl); and —C₁₋₆alkyl substituted by 1, 2 or 3 halogens (for example, F or Cl));
R³ is formula (VIa); and
R^{c} and R^{d} are both H;
or a salt thereof.

In certain other embodiments, the metabolite has a structure according to formula (I), (Ia) or (Ib), wherein,
X is C₁alkylene (i.e. —CH₂—) or C₂alkylene (i.e. —CH₂—CH₂—);
R¹ is selected from the group consisting of H; —C₁₋₄alkyl; and halogen (for example, F or Cl);
R² is selected from the group consisting of H; phenyl optionally substituted by 1, 2 or 3 halogens (for example, F or Cl); and —C₁₋₆alkyl optionally substituted by 1, 2 or 3 halogens (for example, F or Cl); and
R³ is formula (VIa); and
R^{c} and R^{d} are both H;
or a salt thereof.

Preferably, the compound is one of formula (Ia) or (Ib).

Thus, the present invention also provides a compound that has a structure according to formula (I), (Ia) or (Ib), wherein,
X is C₂₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl, optionally substituted by 1, 2 or 3 groups independently selected from halogen; and halogen (for example, F or Cl);
R² is selected from the group consisting of H; phenyl optionally substituted by 1, 2 or 3 groups independently selected from halogen (for example, F or Cl); and —C₁₋₆alkyl optionally substituted by 1, 2 or 3 groups independently selected from halogen (for example, F or Cl); and
R³ is formula (VI)

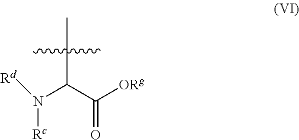

wherein, R^{c} and R^{d} are each independently selected from the group consisting of H, —C₁₋₆alkyl, —C(O)C₁₋₆alkyl and —CH₂-phenyl, wherein said alkyl or said phenyl is optionally substituted by 1, 2 or 3 groups selected from halogen (for example R^{c} and R^{d} are both H); and
R^{g} is selected from the group consisting of H and —C₁₋₆alkyl, wherein said alkyl is optionally substituted by 1, 2 or 3 groups selected from halogen (for example R^{g} is H).

The more advanced metabolite compounds can be used in medical treatments. The invention thus further provides a compound according to formula (I), or a salt or solvate thereof

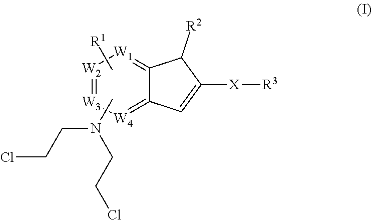

wherein,
X is C₁₋₆alkylene;
W₁, W₂, W₃ and W₄ are each CH, or one of W₁, W₂, W₃ and W₄ is N and the others are CH;
R¹ is selected from the group consisting of H; —C₁₋₄alkyl optionally substituted by 1, 2 or 3 groups independently selected from halogen; and halogen;
R² is selected from the group consisting of H; phenyl optionally substituted by 1, 2 or 3 groups independently selected from halogen; and —C₁₋₆alkyl optionally substituted by 1, 2 or 3 groups independently selected from halogen;
R³ is formula (VI)

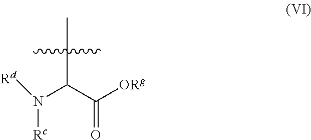

wherein, R^{c} and R^{d} are each independently selected from the group consisting of H, —C₁₋₆alkyl, —C(O)C₁₋₆alkyl and —CH₂-phenyl, wherein said alkyl or said phenyl is optionally substituted by 1, 2 or 3 groups selected from halogen; and
R^{g} is selected from the group consisting of H and —C₁₋₆alkyl, wherein said alkyl is optionally substituted by 1, 2 or 3 groups selected from halogen,
for use as a medicament, for example in the treatment of a cancer selected from the group consisting of multiple myeloma, osteosarcoma, breast cancer, lung cancer, ovarian cancer, leukaemia and lymphoma; for example multiple myeloma, acute myeloid leukemia or lymphoma.

Preferred embodiments of these compounds for use as a medicament are as described above in relation to other aspects of the invention.

In certain preferred embodiments, the further, more advanced metabolite is:
(2S)-2-amino-3-[5-[bis(2-chloroethyl)amino]-1-methyl-benzimidazol-2-yl]propanoic acid (Example compound 17);
(S)-2-amino-4-(5-(bis(2-chloroethyl)amino)-1-methyl-1H-benzo[d]imidazol-2-yl)butanoic acid (Example compound 18);
Ethyl (2S)-2-amino-4-[5-[bis(2-chloroethyl)amino]-1-methyl-benzimidazol-2-yl]butanoate (Example compound 38)
(2S)-2-Amino-4-[5-[bis(2-chloroethyl)amino]-1-phenyl-benzimidazol-2-yl]butanoic acid (Example compound 41); or
(2S)-2-amino-4-[5-[bis(2-chloro-1,1,2,2-tetradeutero-ethyl)amino]-1-methyl-benzimidazol-2-yl]butanoic acid (Example compound 49);
or a salt thereof.

The metabolites described herein may be isolated from cells treated with one or more of the compounds of formula (I) and/or may be prepared using standard organic chemistry techniques. Exemplary procedures for the preparation of metabolites of the present invention are described in the Examples section. For the avoidance of doubt, the metabolites described herein are further examples of compounds of the present invention and may be used in the form of a composition and/or as medicaments in the same manner as described herein for other compounds of formula (I).

The following definitions apply to the terms as used throughout this specification, unless otherwise limited in specific instances.

As used herein, the term “alkyl” means both straight and branched chain saturated hydrocarbon groups. Examples of alkyl groups include methyl, ethyl, n-propyl, iso-propyl, n-butyl, t-butyl, iso-butyl, sec-butyl, pentyl and hexyl groups. Among unbranched alkyl groups, there are preferred methyl, ethyl, n-propyl, iso-propyl and n-butyl groups. Among branched alkyl groups, there may be mentioned t-butyl, i-butyl, 1-ethylpropyl and 1-ethylbutyl groups.

As used herein, the term “cycloalkyl” means a saturated group in a ring system. Examples of cycloalkyl groups include cyclopropyl, cyclobutyl, cyclopentyl and cyclohexyl.

As used herein, the term “halogen” means fluorine, chlorine, bromine or iodine. Fluorine, chlorine and bromine are preferred, and fluorine and chlorine are particularly preferred.

As used herein, the term “heteroaryl comprising 1, 2, 3 or 4 nitrogen, oxygen or sulphur atoms” means an aromatic cyclic group of carbon atoms wherein one, two, three or four of the carbon atoms is/are replaced by one, two, three or four heteroatoms independently selected from nitrogen, oxygen and sulphur (preferably nitrogen and sulphur). Examples of 5-membered heteroaryl comprising 1, 2 or 3 nitrogen and/or sulphur atoms include thiophene, thiazole, isothiazole, pyrrole, pyrroline, pyrazole, pyrazoline, imidazole, imidazoline, triazole and thiadiazole.

### Pharmaceutical Compositions

Compounds, compositions and pharmaceutical compositions according to the invention may be used in the treatment and/or prophylaxis of cancer, reducing tumor growth and/or killing tumor cells. Thus, a compound of the invention may be used for curing and/or prolonging the survival of patients afflicted with cancer diseases. The present invention is especially useful in the treatment and/or prophylaxis of a carcinoma, a sarcoma, a myeloma, a leukemia, a lymphoma or a mixed type of cancer. For example, multiple myeloma, osteosarcoma, breast cancer, lung cancer, ovarian cancer, leukaemia and lymphoma.

While it is possible for a compound according to the invention to be administered alone, it is preferable for it to be present in a composition and particularly in a pharmaceutical composition. Pharmaceutical compositions include those suitable for oral, parenteral (including subcutaneous, intradermal, intraosseous infusion, intramuscular, intravascular (bolus or infusion), intravitreal and intramedullary), intraperitoneal, transmucosal, transdermal, rectal and topical (including dermal, buccal, sublingual and intraocular) administration, although the most suitable route may depend upon for example the condition and disorder of the subject under treatment.

Pharmaceutical compositions of the present invention suitable for oral administration may be presented as discrete units such as capsules, cachets or tablets each containing a predetermined amount of the active ingredient; as a powder or granules; as a solution or a suspension in an aqueous liquid or a non-aqueous liquid; or as an oil-in-water liquid emulsion or a water-in-oil liquid emulsion. A compound of the invention may also be presented as a bolus, electuary or paste.

Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2S, 1988.

Pharmaceutical compositions of the present invention suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. Preferably the formulations may be presented in unit dosage or divided dosage containers, for example sealed ampoules and vials. The formulation may be stored in a freeze-dried (lyophilised) condition requiring only the addition of the sterile liquid carrier, for example saline or water-for-injection, immediately prior to use. Extemporaneous injection and infusion solutions and suspensions may be prepared from sterile powders, granules or other dry composition. Exemplary compositions for parenteral administration include injectable solutions or suspensions which can contain, for example, suitable non-toxic, parenterally acceptable solutes, diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor.

Pharmaceutical compositions of the present invention suitable for nasal, aerosol or inhalation administration include solutions in saline, which can contain, for example, benzyl alcohol or other suitable preservatives, absorption promoters to enhance bioavailability, and/or other solubilizing or dispersing agents such as those known in the art.

Pharmaceutical compositions of the present invention suitable for rectal administration may be presented as a suppository with the usual carriers such as cocoa butter, synthetic glyceride esters or polyethylene glycol. Such carriers are typically solid at ordinary temperatures, but liquefy and/or dissolve in the rectal cavity to release the drug.

Pharmaceutical compositions of the present invention suitable for topical administration in the mouth, for example buccally or sublingually, include lozenges comprising the active ingredient in a flavoured basis such as sucrose and acacia or tragacanth, and pastilles comprising the active ingredient in a basis such as gelatin and glycerine or sucrose and acacia. Exemplary compositions for topical administration include a topical carrier such as Plastibase (mineral oil gelled with polyethylene).

Preferred unit dosage compositions are those containing an exploratory dose or therapeutic dose, or an appropriate fraction thereof, of a compound of the invention.

In preferred embodiments, a composition of the invention consists essentially of a compound of the invention and at least one pharmaceutically acceptable excipient.

It should be understood that in addition to the ingredients particularly mentioned above, the compositions for use in this invention may include other agents conventional in the art having regard to the type of composition in question.

The compositions of the invention may comprise one or more further therapeutic agents. Examples of further therapeutic agents that may be present in a composition of the present invention include, but are not limited, to steroids (prednisone and dexamethasone), checkpoint inhibitors (e.g. CTLA-4 inhibitors and PD-1/PD-L1 inhibitors), nuclear transport inhibitors (e.g. selinexor), anti-apoptotic inhibitors (e.g. venetoclax), adoptive cell therapy (e.g., tumor-Infiltrating lymphocyte (TIL) therapy, Natural Killer (NK) cell therapy, chimeric antigen receptor (CAR) T-cell therapy, and engineered T-cell receptor therapy), bi-specific T-cell engagers (BiTEs), immunomodulatory imide drugs (IMiDs) (e.g. thalidomide, lenalidomide and pomalidomide), proteasome inhibitors (PIs) (e.g. bortezomib, carfilzomib, ixazomib and marizomib), histone deacetylase (HDAC) inhibitors (e.g. panobinostat), a peptide drug conjugate (PDC) other than a compound of the present invention (e.g. melflufen), and conventional chemotherapy, such as alkylators (e.g. melphalan, cyclophosphamide) and DNA intercalators (e.g. doxorubicin).

### Treatments

As mentioned above, compounds of the present invention are peptide drug conjugates (PDCs). The invention provides a compound according to the invention, or a composition comprising a compound according to the invention together with a pharmaceutically acceptable carrier, for use as a medicament.

In particular, compounds and compositions of the present invention find use in the treatment and/or prophylaxis of cancer. Particular examples of cancers that may be treated or prevented by administering a compound or composition of the invention include carcinoma, a sarcoma, a myeloma, a leukemia, a lymphoma or a mixed type of cancer. Exemplary cancers that may be treated or prevented by administering a compound or composition of the invention include, but are not limited to, hematologic/blood cell cancers such as a leukemia (for example, acute lymphoblastic leukemia including adult and childhood acute lymphoblastic leukemia; acute myeloid leukemia including adult and childhood acute myeloid leukemia; chronic lymphocytic leukemia such as B Cell chronic lymphocytic leukemia; chronic myelogenous leukemia; and hairy cell leukemia); lymphoma (e.g., AIDS-related lymphoma; cutaneous T-cell lymphoma; Hodgkin's lymphoma including adult and childhood Hodgkin's lymphoma and Hodgkin's lymphoma during pregnancy; non-Hodgkin's lymphoma including adult and childhood non-Hodgkin's lymphoma and non-Hodgkin's lymphoma during pregnancy; mycosis fungoides; Sezary syndrome; Waldenstrom's macroglobulinemia; primary mediastinal large B cell lymphoma; mantle cell lymphoma; diffuse large B cell lymphoma; and primary central nervous system lymphoma); and other hematologic cancers (for example, chronic myeloproliferative disorders; multiple myeloma/plasma cell neoplasm; myelodysplastic syndromes; and myelodysplastic/myeloproliferative disorders); osteosarcoma, ovarian cancer, breast cancer; lung cancer; glioblastoma; retinoblastoma and metastases of the aforementioned cancers.

The present inventors have found that the compounds of the present invention are particularly cytotoxic towards hematological cancer cells. Thus, the compounds of the present invention are particularly beneficial in the treatment or prevention of one or more of the aforementioned hematological cancers.

Furthermore, the compounds may also be used in the treatment of solid tumors. Compounds and compositions of the invention also find utility in a method of treating or preventing a disease or disorder, said method comprising a step of administering a compound of the invention, or a composition of the invention, to a subject in need thereof. As such, a compound or composition of the invention may be administered to a subject suffering, or at risk of developing, a cancer, particularly a haematological cancer.

Compounds of the invention also find use in the manufacture of a medicament, particularly use in the manufacture of a medicament to be administered to a subject suffering, or at risk of developing, a cancer, particularly a haematological cancer.

The amount of a compound of the invention which is required to achieve a therapeutic effect will vary with particular route of administration and the characteristics of the subject under treatment, for example the species, age, weight, sex, medical conditions, the particular disease or condition and its severity, and other relevant medical and physical factors. An ordinarily skilled physician can readily determine and administer an effective amount of the compound of the invention required for treatment or prophylaxis of a disease or condition.

The compound of the invention, or salt and/or solvate thereof, may be administered daily (including several times daily), every second or third day, weekly, every second, third or fourth week or even as a high single dose depending on the subject and disease or disorder to be treated.

Preferably, a compound of the invention, or metabolite of the invention, or salt and/or solvate thereof (excluding the mass of any counterion or solvent), may be administered in an amount of about 1 mg to 150 mg per administration. For example, 1 mg, 2 mg, 3 mg, 5 mg, 10 mg, 15 mg, 20 mg, 25 mg, 40 mg, 50 mg, 60 mg, 70 mg, 80 mg, 90 mg, 100 mg, 110 mg, 120 mg, 130 mg, 140 mg or 150 mg. Alternatively, a compound of the invention, or salt and/or solvate thereof (excluding the mass of any counterion or solvent), may be administered in a single high dose. A single high dose may be about 150 mg to 800 mg. For example, 150 mg, 200 mg, 300 mg, 400 mg, 500 mg, 600 mg, 700 mg or 800 mg.

Whilst a compound of the invention may be used as the sole active ingredient in the present invention, it is also possible for it to be used in combination with one or more further therapeutic agent(s), and the use of such combinations provides one preferred embodiment of the invention. Such further therapeutic agents may be agents useful in the treatment or prophylaxis of cancer, or other pharmaceutically active materials. Such agents are known in the art. Examples of further therapeutic agents suitable for use in the present invention are described herein.

The one or more further therapeutic agent(s) may be used simultaneously, sequentially or separately with/from the administration of a compound of the invention. The individual components of such combinations can be administered separately at different times during the course of therapy or concurrently in divided or single combination forms.

Preferred unit dosage compositions for use according to the invention are those containing an effective dose, or an appropriate fraction thereof, of a compound of the invention.

### Kits

The present invention provides a kit comprising a compound of formula (I), one or more pharmaceutically acceptable excipients, and optionally one or more further therapeutic agents. Examples of such further therapeutic agents include those described herein as being suitable for use in the present invention, and being optionally present in a pharmaceutical composition of the invention as a further therapeutic agent.

Kits of the present invention find use in the treatment and/or prophylaxis of cancers, particularly haematological cancers.

In certain embodiments the kit comprises one or more containers and may also include sampling equipment, for example, bottles, bags (such as intravenous fluid bags), vials, syringes, and test tubes. Other components may include needles, diluents, wash reagents and buffers. Usefully, the kit may include at least one container comprising a pharmaceutically acceptable organic solvent or a pharmaceutically-acceptable buffer, such as phosphate-buffered saline, Ringer's solution and dextrose solution.

Preferably, the kit of the invention comprises instructions, for example instructions that instruct a user to admix a stated amount of compound or composition of the present invention with a stated amount physiologically acceptable aqueous solvent or diluent, pharmaceutically acceptable organic solvent, and/or optional one or more further therapeutic agents. Such instructions may also provide guidance on the storage conditions and/or administration instructions.

For the avoidance of doubt, the compound or composition of the present invention, optional physiologically acceptable aqueous solvent or diluent, optional one or more pharmaceutically acceptable organic solvents, and optional one or more further therapeutic agents, are present in a kit according to the present invention in a form and quantity suitable for the preparation of a pharmaceutical preparation according to the invention. The skilled person can readily determine a quantity of a compound or composition of the present invention, physiologically acceptable aqueous solvent or diluent, pharmaceutically acceptable organic solvent, and optional one or more further therapeutic agents, suitable for the use according the present invention.

### EQUIVALENTS

The invention has been described broadly and generically herein. Those of ordinary skill in the art will readily appreciate that all parameters, dimensions, materials, and configurations described herein are meant to be exemplary and that the actual parameters, dimensions, materials, and/or configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the foregoing embodiments are presented by way of example only and that, within the scope of the appended claims and equivalents thereto, the invention may be practiced otherwise than as specifically described and claimed. The present invention is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present invention. Further, each of the narrower species and subgeneric groupings falling within the generic disclosure also form part of the invention. This includes the generic description of the invention with a proviso or negative limitation removing any subject matter from the genus, regardless of whether or not the excised material is specifically recited herein.

### INCORPORATION BY REFERENCE

The contents of the articles, patents, and patent applications, and all other documents and electronically available information mentioned or cited herein, are hereby incorporated by reference in their entirety to the same extent as if each individual publication was specifically and individually indicated to be incorporated by reference. The applicant reserves the right physically to incorporate into this application any and all materials and information from any such articles, patents, patent applications, or other physical and electronic documents.

The following Examples illustrate the invention.

### Examples

### Example Compounds

The compounds of the invention may be prepared according to known methods for those skilled in the art. Other reaction schemes, as well as a variety of different solvents, temperatures and other reaction conditions, could be readily devised by those skilled in the art.

Intermediates 1-4 were synthesized according to the general Scheme 1. First, amidation reaction was performed between the corresponding protected amino acid and the aniline. The obtained product was cyclized under acidic conditions to form the benzimidazole heterocycle and subsequently intermediates 1 (n=1) and 3 (n=0). BOC protection followed by saponification afforded both intermediates 3 (n=1) and 4 (n=0).

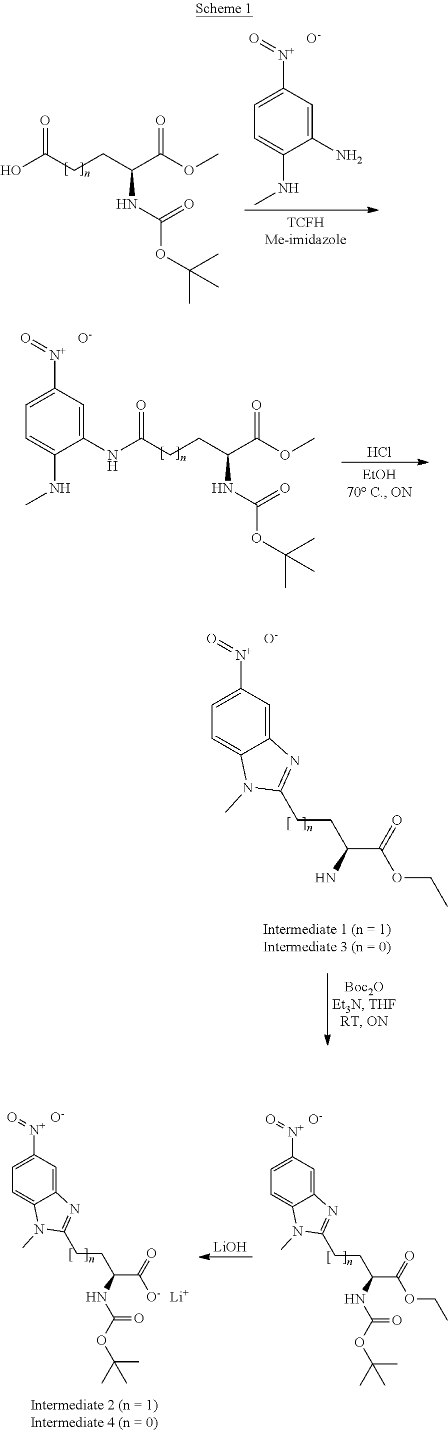

Peptide coupling with TCFH between intermediate (2 or 4) and the corresponding protected amino acid furnished the desired dipeptide. Reduction of the nitro group was performed with Pd/C under pressured atmosphere of hydrogen. The formed aniline was alkylated under acidic conditions with ethylene oxide. Mesylation followed by chlorine displacement provided the nitrogen mustard moiety. Finally, removal of the BOC protected group with HCl or TFA furnished the desired analogues (Scheme 2).

Intermediate 2 and 4 can also be synthesized according to Scheme 13.

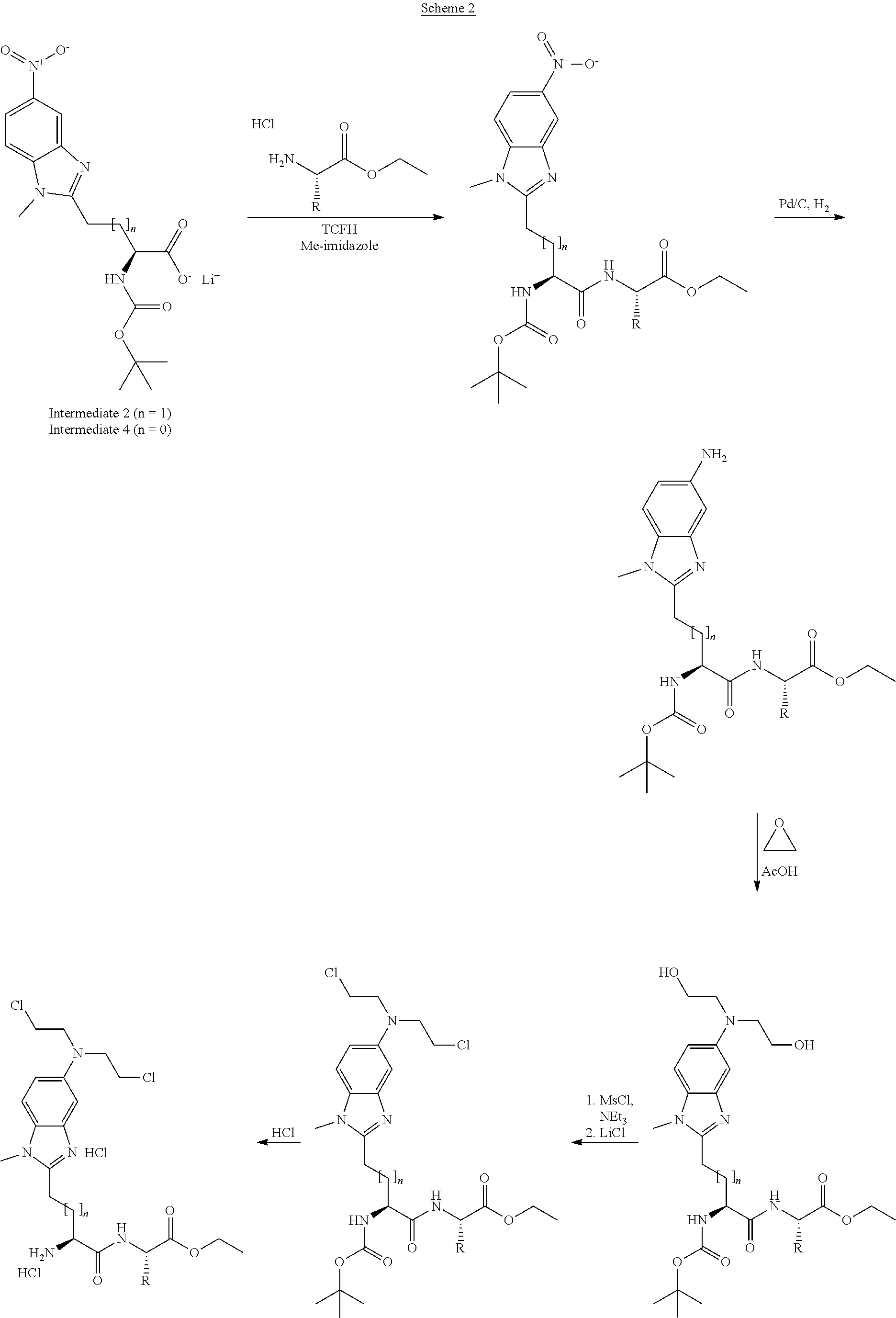

Example compounds 1 to 6 were synthesized according to the synthetic route depicted in Scheme 2. Example compounds 13 to 15, 19, 20, 22 to 36, 39, 40 to 42, and 44 to 51 were synthesized as described in detail below. A similar strategy as above was applied to the synthesis of Example compounds 7 to 12, which have a benzimidazole substituent at the C-terminus of the dipeptide (Scheme 3). Example compounds 16, 21, 37 and 43 were synthesized as described in detail below.

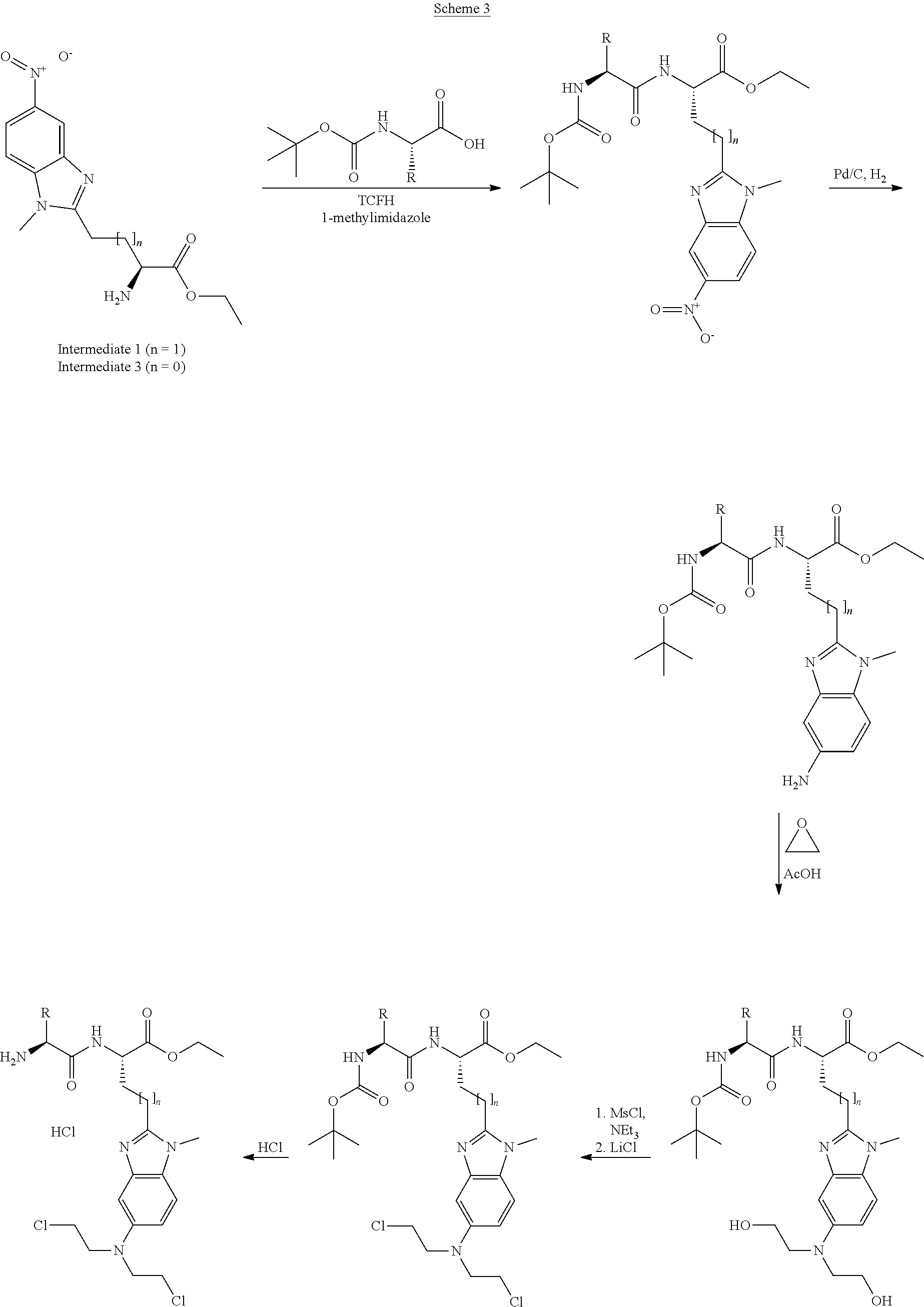

### Materials and Methods

### General Methods A

Preparative HPLC was performed on a Gilson HPLC system using a Kinetex XB C₁₈ (5 μm, 21×100 mm) column with 0.1% TFA in MilliQ H₂O/CH₃CN as mobile phase (acidic conditions) (flow 25 mL/min, gradient over 15 min). Fractions were collected based on the UV-signal. Analyses were performed on an Agilent Series 1100 system by two methods using either a Kinetex XB C18 (2.6 μm, 3.0×50 mm) column with 0.1% TFA in MilliQ H₂O/CH₃CN as mobile phase (acidic conditions) or on a Kinetex EVO C₁₈ (2.6 μm, 3.0×50 mm) column with 10 mM NH₄HCO₃ (pH10) in MilliQ H₂O/CH₃CN as mobile phase (basic conditions) (flow 1 mL/min). Electrospray ionization mass spectrometry (ESI-MS) was performed using an Agilent 1100 Series Liquid Chromatograph/Mass Selective Detector (MSD) to obtain the pseudo molecular [M+H]⁺ ion of the target molecules.

Nuclear Magnetic Resonance (NMR) spectra were recorded at 25° C. on a Varian Inova 600 MHz instrument equipped with a 5 mm triple resonance probe.

General methods B

General methods B were used for certain examples. Flash chromatography was performed on silica gel 60A, 40-63 μm. Preparative HPLC was performed on a Gilson system equipped with a UV detector using an ACE 5 C₈ Prep, 100×21.2 mm column. Analytical HPLC-MS was performed using an Agilent 1100 series Liquid Chromatograph/Mass Selective Detector (MSD) (Single Quadrupole) equipped with an electrospray interface and a UV diode array detector. Analyses were performed by two methods using either an ACE 3 C₈ (3.0×50 mm) column with a gradient of acetonitrile in 0.1% aqueous TFA over 3 min and a flow of 1 mL/min, or an Xbridge C₁₈ (3.0×50 mm) column with a gradient of acetonitrile in 10 mM ammonium bicarbonate over 3 min and a flow of 1 mL/min. ¹H-NMR spectra were recorded on a Bruker 400 MHz instrument at 25° C.

Common General methods

For Example compounds prepared as hydrochloride salts, HCl (e.g. 4M in 1,4-dioxane) was added to the combined pure fractions from the preparative chromatography in order to replace the trifluoroacetic acid with HCl.

The compounds have been named using software from Biovia, Dotmatics and PerkinElmer (ChemDraw). In addition, the commercial names or trivial names were used for the commercial starting materials and reagents.

Spectra were processed using MestReNova, supplied by Mestrelab Research S.L. Chemical shifts are reported in ppm (δ) using the residual solvent as internal standard. Peak multiplicities given in Hz are expressed as follow: s, singlet; d, doublet; dd, doublet of doublets; ddd, doublet of doublet of doublets; t, triplet; dt, doublet of triplets; q, quartet; dq, doublet of quartets; p, pentet; h, heptet; m, multiplet; br s, broad singlet. One downfield proton (around 14.5-14.9 ppm) is sometimes out of range and not always included in the multiplet report.

### Intermediate 1: Ethyl (2S)-2-amino-4-(1-methyl-5-nitro-benzimidazol-2-yl)butanoate

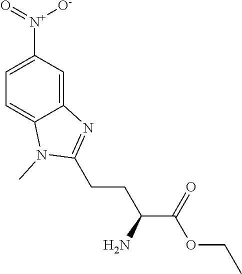 [SMILES: CCOC(=O)[C@@H](N)CCc1nc2cc([N+](=O)[O-])ccc2n1C]

To a solution of (4S)-4-(tert-butoxycarbonylamino)-5-methoxy-5-oxo-pentanoic acid (7.46 g, 28.6 mmol), N1-methyl-4-nitro-benzene-1,2-diamine (5.01 g, 30.0 mmol), 1-methylimidazole (4.78 mL, 60.0 mmol) in MeCN (220 mL) was added [chloro(dimethylamino)methylene]-dimethyl-ammonium; hexafluorophosphate (8.81 g, 31.4 mmol) at room temperature [slight exotherm]. The mixture was stirred at room temperature for 1 h and the volatiles were evaporated under reduced pressure. The crude was dissolved in DCM (400 mL) and washed with a 2N HCl:water 1:4 solution (200 mL). The organic phase was separated, dried over MgSO₄, filtered and concentrated to afford a brown solid. The crude product, methyl (2S)-2-(tert-butoxycarbonylamino)-5-[2-(methylamino)-5-nitro-anilino]-5-oxo-pentanoate (13.4 g, 28.4 mmol, yield: 99%), was used in the next step without further purification. HPLC purity: 87%; MS (ESI+) m/z=354 [M+H-tBu]⁺.

To a solution of methyl (2S)-2-(tert-butoxycarbonylamino)-5-[2-(methylamino)-5-nitro-anilino]-5-oxo-pentanoate (11.7 g, 28.5 mmol) in EtOH (400 mL) was added hydrogen chloride (12.0 mol/L, 42.3 mL, 507 mmol) and the mixture was stirred at 70° C. overnight. The reaction was evaporated under reduced pressure and water (500 mL) was added. The aqueous phase was washed with EtOAc (2×200 mL). The organics were discarded. The aqueous phase was mixed with EtOAc (500 mL) and neutralized with solid K₂CO₃ until pH 9-10. The aqueous phase was extracted with EtOAc (2×300 mL). The organics were dried over MgSO₄, filtered and evaporated under reduced pressure giving the crude product, ethyl (2S)-2-amino-4-(1-methyl-5-nitro-benzimidazol-2-yl)butanoate (Intermediate 1), which was used in the next step without further purifications. HPLC purity: 83%; MS (ESI⁺) m/z=307 [M+H]⁺.

### Intermediate 2: Lithium(2S)-2-(tert-butoxycarbonylamino)-4-(1-methyl-5-nitro-benzimidazol-2-yl)butanoate

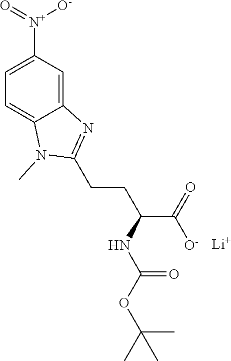 [SMILES: Cn1c(CC[C@H](NC(=O)OC(C)(C)C)C(=O)[O-])nc2cc([N+](=O)[O-])ccc21 ; [Li+]]

To a solution of crude ethyl (2S)-2-amino-4-(1-methyl-5-nitro-benzimidazol-2-yl)butanoate (Intermediate 1) (5.73 g, 18.7 mmol) in THF (100 mL) was added triethylamine (3.13 mL, 22.4 mmol) and di-tert-butyl dicarbonate (4.49 g, 20.6 mmol) and the mixture was stirred at room temperature overnight. The crude was concentrated under reduced pressure, dissolved with EtOAc (200 mL) and washed with 0.5N HCl (200 mL). The phases were separated and the aqueous phase was extracted with EtOAc (2×50 mL). The organics were dried over MgSO₄, filtered and evaporated under reduced pressure. Et₂O (150 mL) was added and a solid started to precipitate. The flask was cooled to 0° C. and let stand for 30 min. The solid was collected by filtration and washed with cold Et₂O (3×50 mL). The solid was dried under reduced pressure to afford ethyl (2S)-2-(tert-butoxycarbonylamino)-4-(1-methyl-5-nitro-benzimidazol-2-yl)butanoate (4.70 g, 11.6 mmol, yield over 3 steps: 55%), as a light yellow solid. HPLC purity: 96%, MS (ESI+) m/z=407 [M+H]⁺.

To a solution of ethyl (2S)-2-(tert-butoxycarbonylamino)-4-(1-methyl-5-nitro-benzimidazol-2-yl)butanoate (1.47 g, 3.62 mmol) in THF (20 mL) and MeOH (20 mL) was added water (5 mL) and LiOH—H₂O (0.159 g, 3.80 mmol) and the mixture was stirred at room temperature overnight. The crude product, lithium; (2S)-2-(tert-butoxycarbonylamino)-4-(1-methyl-5-nitro-benzimidazol-2-yl)butanoate (Intermediate 2), was used in the next step without further purifications. HPLC purity: 100%; MS (ESI+) m/z=379 [M(carboxylic acid)+H]⁺.

### Intermediate 3: Ethyl (2S)-2-amino-3-(1-methyl-5-nitro-benzimidazol-2-yl)propanoate

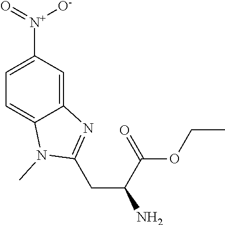 [SMILES: CCOC(=O)[C@@H](N)Cc1nc2cc([N+](=O)[O-])ccc2n1C]

Intermediate 3 was synthesized following the same procedure as for Intermediate 1 but using (3S)-3-(tert-butoxycarbonylamino)-4-methoxy-4-oxo-butanoic acid as starting material: Step 1: The crude product, methyl (2S)-2-(tert-butoxycarbonylamino)-4-[2-(methylamino)-5-nitro-anilino]-4-oxo-butanoate, was isolated as a brown solid (71% yield). HPLC purity: 71%; m/z=341 (M+H-tBu)⁺.

Step 2: Ethyl (2S)-2-amino-3-(1-methyl-5-nitro-benzimidazol-2-yl)propanoate (Intermediate 3) was isolated as a yellow-orange solid (98% yield). HPLC purity: 91%; m/z=293 (M+H)⁺.

### Intermediate 4: Lithium; (2S)-2-(tert-butoxycarbonylamino)-3-(1-methyl-5-nitro-benzimidazol-2-yl)propanoate

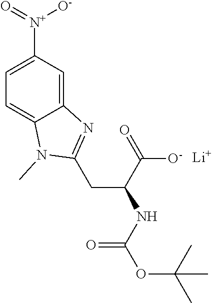 [SMILES: Cn1c(C[C@H](NC(=O)OC(C)(C)C)C(=O)[O-])nc2cc([N+](=O)[O-])ccc21 ; [Li+]]

Intermediate 4 was synthesized following the same procedure as for Intermediate 2 but using ethyl (2S)-2-amino-3-(1-methyl-5-nitro-benzimidazol-2-yl)propanoate (Intermediate 3) as starting material.

Step 1: The crude product, ethyl (2S)-2-(tert-butoxycarbonylamino)-3-(1-methyl-5-nitro-benzimidazol-2-yl)propanoate, was isolated as a brown solid (83% yield). HPLC purity: 97%; m/z=393 [M+H]⁺.

Step 2: Lithium; (2S)-2-(tert-butoxycarbonylamino)-3-(1-methyl-5-nitro-benzimidazol-2-yl)propanoate (intermediate 4) was isolated as a yellow-orange solid (quantitative yield). HPLC purity: 91%; m/z=365 [M(carboxylic acid)+H]⁺.

### Example 1: Ethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 1)

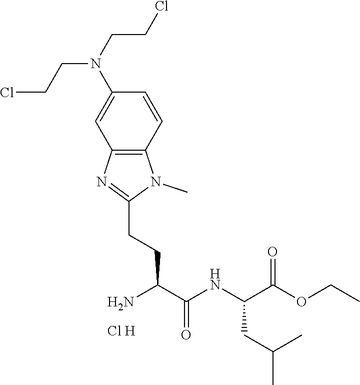

Step 1: To a solution of ethyl (2S)-2-amino-4-methyl-pentanoate; hydrochloride (0.581 g, 2.97 mmol), lithium; (2S)-2-(tert-butoxycarbonylamino)-4-(1-methyl-5-nitro-benzimidazol-2-yl)butanoate (Intermediate 2) (1.04 g, 2.70 mmol) and 1-methylimidazole (0.452 mL, 5.67 mmol) in MeCN (30 mL) was added [chloro(dimethylamino)methylene]-dimethyl-ammonium; hexafluorophosphate (0.834 g, 2.97 mmol) at room temperature. The mixture was stirred at room temperature for 1 h. The reaction was quenched with citric acid solution (10% in water) and the volatiles were evaporated. The crude was diluted with water and extracted with EtOAc (×3). The organics were dried over MgSO₄, filtered, and evaporated under reduced pressure. The crude was purified by silica chromatography (DCM->DCM/MeOH (10%)) to afford ethyl (2S)-2-[[(2S)-2-(tert-butoxycarbonylamino)-4-(1-methyl-5-nitro-benzimidazol-2-yl)butanoyl]amino]-4-methyl-pentanoate as a light yellow foam (1.27 g, 2.44 mmol, 90% yield). HPLC purity: 96%; MS (ESI+) m/z=520 [M+H]⁺.

Step 2: Ethyl (2S)-2-[[(2S)-2-(tert-butoxycarbonylamino)-4-(1-methyl-5-nitro-benzimidazol-2-yl)butanoyl]amino]-4-methyl-pentanoate (1.27 g, 0.00244 mol) was dissolved in EtOH (30 mL) and placed in a H-reactor. Pd/C (10%, 0.130 g, 0.000122 mol) was added and the reactor was sealed. A nitrogen purge was conducted. H₂ was charged to the reactor at 3 bar and the reaction was stirred at 35° C. for 1.5 h.

Hydrogen was vented with a nitrogen purge. Catalyst was removed by filtration through Celite. The solvent was evaporated and the crude product, ethyl (2S)-2-[[(2S)-4-(5-amino-1-methyl-benzimidazol-2-yl)-2-(tert-butoxycarbonylamino)butanoyl]amino]-4-methyl-pentanoate (0.670 g, 1.37 mmol, 85% yield), was isolated and used in the next step without further purification. HPLC purity: 93%; MS (ESI+) m/z=490 [M+H]⁺.

Step 3: Ethyl (2S)-2-[[(2S)-4-(5-amino-1-methyl-benzimidazol-2-yl)-2-(tert-butoxycarbonylamino)butanoyl]amino]-4-methyl-pentanoate (670 mg, 0.00137 mol) was dissolved in acetic acid (5 mL) and oxirane (2.30 mol/L, 5.95 mL, 0.0137 mol) was added and the reaction was stirred at room temperature for 24 h. The volatiles were removed under vacuum, the crude material was dissolved in DCM, and washed with saturated aqueous NaHCO₃. The organic layer was washed with water and brine, passed through a phase separator and concentrated. The crude was purified by silica chromatography using DCM->DCM/MeOH (10%) affording ethyl (2S)-2-[[(2S)-4-[5-[bis(2-hydroxyethyl)amino]-1-methyl-benzimidazol-2-yl]-2-(tert-butoxycarbonylamino)butanoyl]amino]-4-methyl-pentanoate (0.340 g, 0.589 mmol, 43% yield) as a pale-white foam. HPLC purity: 96%; MS (ESI+) m/z=578 [M+H]⁺.

Step 4: To a solution of ethyl (2S)-2-[[(2S)-4-[5-[bis(2-hydroxyethyl)amino]-1-methyl-benzimidazol-2-yl]-2-(tert-butoxycarbonylamino)butanoyl]amino]-4-methyl-pentanoate (340 mg, 0.589 mmol) and triethylamine (0.238 g, 2.35 mmol) in DCM (10 mL) was added methanesulfonyl chloride (0.141 mL, 1.82 mmol). The reaction mixture was stirred at room temperature for 20 min and then washed with a mixture of NaHCO₃ (sat): water (2/5) (20 mL), 0.3M HCl (20 mL) and brine (20 mL). The organic phase was dried over MgSO₄, filtered, and evaporated under reduced pressure.

The crude mixture (mesylated intermediate) and lithium chloride (0.374 g, 8.83 mmol) in DMF (10 mL) were heated to 60° C. for 2.5 h. The reaction mixture was diluted with a mixture of toluene (40 mL) and EtOAc (40 mL), then washed with brine (3×50 mL), dried over MgSO₄, filtered and the solvent was evaporated to give a crude residue. The crude was purified by flash chromatography using DCM->DCM/MeOH(5%) as eluent. The final compound, ethyl (2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate, was obtained as light yellow foam (0.308 g, 0.481 mmol, 82% yield). HPLC of mesylated intermediate: MS (ESI+) m/z=734 [M+H]⁺. HPLC purity of desired product: 96%; MS (ESI+) m/z=614 [M+H]⁺.

Step 5: To a solution of ethyl (2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate (308 mg, 0.476 mmol) in EtOH (15 mL) was added hydrogen chloride in dioxane (4 M) (4.00 mol/L, 1.79 mL, 7.14 mmol) and the mixture was stirred at room temperature overnight. The solvents were evaporated, giving the crude product which was stirred in Et₂O. The off-white solid formed was filtered, washed with Et₂O (2×10 mL) and washed with EtOAc (2 mL) and dried over vacuum to afford the final product, ethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 1) as an off-white solid (0.170 g, 0.272 mmol, yield: 57%). HPLC purity: 95%, MS (ESI+) m/z=514 [M+H]⁺.

¹H NMR (600 MHz, DMSO-d6) δ 14.86 (br s, 1H), 9.31 (d, J=7.0 Hz, 1H), 8.60 (d, J=5.3 Hz, 3H), 7.75 (d, J=9.2 Hz, 1H), 7.15 (dd, J=9.4, 2.4 Hz, 1H), 6.93 (d, J=2.4 Hz, 1H), 4.34-4.27 (m, 1H), 4.15-4.06 (m, 3H), 3.91 (s, 3H), 3.83 (t, J=6.8 Hz, 4H), 3.78 (t, J=6.9 Hz, 4H), 3.43-3.30 (m, 2H), 2.41-2.25 (m, 2H), 1.81-1.71 (m, 1H), 1.64 (ddd, J=13.7, 10.4, 5.1 Hz, 1H), 1.54 (ddd, J=13.8, 9.2, 4.7 Hz, 1H), 1.18 (t, J=7.1 Hz, 3H), 0.92 (d, J=6.6 Hz, 3H), 0.88 (d, J=6.5 Hz, 3H).

Example 1 has also been synthesized according to the procedure described for Example 46.

### Example 2: Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate; dihydrochloride (Example compound 2)

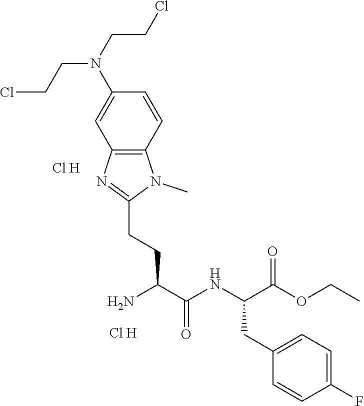

The title compound was prepared in 5 steps using the method described in Example 1.

Step 1: Ethyl (2S)-2-[[(2S)-2-(tert-butoxycarbonylamino)-4-(1-methyl-5-nitro-benzimidazol-2-yl)butanoyl]amino]-3-(4-fluorophenyl)propanoate (5.14 g, 8.99 mmol, 86% yield) was isolated as a white solid. HPLC purity: 99%; MS (ESI+) m/z=572 [M+H]⁺.

¹H NMR (600 MHz, DMSO-d6, presence of rotamers) δ 8.44 (d, J=2.3 Hz, 1H), 8.37 (d, J=7.5 Hz, 1H), 8.15 (dd, J=8.9, 2.2 Hz, 1H), 7.75 (d, J=9.0 Hz, 1H), 7.26 (dd, J=8.4, 5.6 Hz, 2H), 7.10-7.01 (m, 3H), 4.51-4.44 (m, 1H), 4.10-4.04 (m, 1H), 4.02 (t, J=7.1 Hz, 2H), 3.79 (s, 3H), 3.06-2.92 (m, 2H), 2.92-2.83 (m, 2H), 2.13-2.02 (m, 2H), 1.37 (s, 8H, major rotamer), 1.21 (s, 1H, minor rotamer), 1.08 (t, J=7.1 Hz, 3H).

Step 2: The crude product ethyl (2S)-2-[[(2S)-4-(5-amino-1-methyl-benzimidazol-2-yl)-2-(tert-butoxycarbonylamino)butanoyl]amino]-3-(4-fluorophenyl)propanoate (4.83 g, 0.00892 mol, 93% yield) was isolated as a light yellow foam. The crude was used directly in the next step. HPLC purity: 96%; MS (ESI+) m/z=542 [M+H]⁺.

¹H NMR (600 MHz, DMSO-d6, presence of rotamers) δ 8.49 (d, J=7.5 Hz, 1H), 7.28 (dd, J=8.5, 5.7 Hz, 2H), 7.14-7.03 (m, 4H), 6.69 (d, J=2.0 Hz, 1H), 6.53 (dd, J=8.5, 2.1 Hz, 1H), 4.68 (s, 2H), 4.49-4.41 (m, 1H), 4.07-3.98 (m, 3H), 3.58 (s, 3H), 3.07-2.94 (m, 2H), 2.82-2.70 (m, 2H), 2.08-1.98 (m, 1H), 1.98-1.87 (m, 1H), 1.37 (s, 8H, major rotamer), 1.19 (s, 1H, minor rotamer), 1.13-1.03 (m, 3H).

Step 3: The desired product, ethyl (2S)-2-[[(2S)-4-[5-[bis(2-hydroxyethyl)amino]-1-methyl-benzimidazol-2-yl]-2-(tert-butoxycarbonylamino)butanoyl]amino]-3-(4-fluorophenyl)propanoate (3.00 g, 4.76 mmol, 54% yield) was isolated as a white solid. HPLC purity: 100%, MS (ESI+) m/z=630 [M+H]⁺.

¹H NMR (600 MHz, DMSO-d6, presence of rotamers) δ 8.47-8.43 (m, 1H), 7.30-7.23 (m, 3H), 7.13 (d, J=8.1 Hz, 1H), 7.06 (t, J=8.8 Hz, 2H), 6.82 (d, J=2.4 Hz, 1H), 6.73-6.69 (m, 1H), 4.71 (t, J=5.4 Hz, 2H), 4.46 (q, J=7.4 Hz, 1H), 4.07-3.98 (m, 3H), 3.61 (s, 3H), 3.53 (q, J=6.2 Hz, 4H), 3.42-3.35 (m, 4H), 3.07-2.93 (m, 2H), 2.80-2.74 (m, 2H), 2.09-2.00 (m, 1H), 2.00-1.92 (m, 1H), 1.37 (s, 8H, major rotamer), 1.20 (s, 1H, minor rotamer), 1.11-1.05 (m, 3H).

Step 4: The crude product was purified using Buchi automatic flash system (80 g column, DCM/MeOH, 100:0 to 92:8) to afford ethyl (2S)-2-[[(2S)-4-[5-[bis(2-chloroethyl)amino]-1-methyl-benzimidazol-2-yl]-2-(tert-butoxycarbonylamino)butanoyl]amino]-3-(4-fluorophenyl)propanoate (1.60 g, 2.40 mmol, 50% yield) as a light yellow solid. HPLC purity: 97%, MS (ESI+) m/z=666 [M+H]⁺.

Step 5: Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate; dihydrochloride (Example compound 2) (1.46 g, 2.28 mmol, yield: 95%) was isolated as an off-white solid. HPLC purity: 97%, MS (ESI+) m/z=566 [M+H]⁺.

¹H NMR (600 MHz, DMSO-d6) δ 14.75 (br s, 1H), 9.42-9.38 (m, 1H), 8.56 (s, 3H), 7.75 (d, J=9.2 Hz, 1H), 7.35 (dd, J=8.5, 5.6 Hz, 2H), 7.18-7.09 (m, 3H), 6.93 (d, J=2.3 Hz, 1H), 4.52 (q, J=7.1 Hz, 1H), 4.11-4.00 (m, 3H), 3.90 (s, 3H), 3.85-3.82 (m, 4H), 3.79-3.76 (m, 4H), 3.41-3.26 (m, 2H), 3.10-3.01 (m, 2H), 2.36-2.27 (m, 2H), 1.11 (t, J=7.1 Hz, 3H).

### Example 3: Ethyl (2S)-2-[[(2S)-2-amino-4-[5-[bis(2-chloroethyl)amino]-1-methyl-benzimidazol-2-yl]butanoyl]amino]-3-methyl-butanoate; dihydrochloride (Example compound 3)

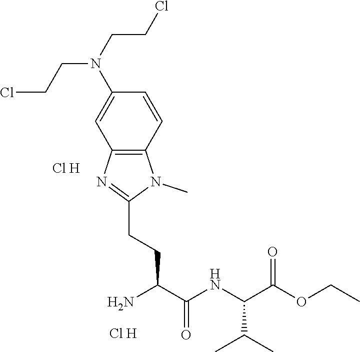

The title compound was prepared in 5 steps using the method described in Example 1.

Step 1: Ethyl (2S)-2-[[(2S)-2-(tert-butoxycarbonylamino)-4-(1-methyl-5-nitro-benzimidazol-2-yl)butanoyl]amino]-3-methyl-butanoate (1.27 g, 2.27 mmol, 84% yield) was isolated as a light yellow foam. HPLC purity: 93%, MS (ESI+) m/z=506 [M+H]⁺.

Step 2: The crude product, ethyl (2S)-2-[[(2S)-4-(5-amino-1-methyl-benzimidazol-2-yl)-2-(tert-butoxycarbonylamino)butanoyl]amino]-3-methyl-butanoate (1.2 g, 97% yield), was obtained as a beige solid, which was used in the next step without further purification. HPLC purity: 97%, MS (ESI+) m/z=476 [M+H]⁺.

Step 3: Ethyl (2S)-2-[[(2S)-4-[5-[bis(2-hydroxyethyl)amino]-1-methyl-benzimidazol-2-yl]-2-(tert-butoxycarbonylamino)butanoyl]amino]-3-methyl-butanoate (1.38 g, 98% yield) was obtained as a brown oil, which solidified upon standing. The compound was used in the next step without further purification. HPLC purity: 98%, MS (ESI+) m/z=564 [M+H]⁺.

Step 4: The desired compound, ethyl (2S)-2-[[(2S)-4-[5-[bis(2-chloroethyl)amino]-1-methyl-benzimidazol-2-yl]-2-(tert-butoxycarbonylamino)butanoyl]amino]-3-methyl-butanoate (836 mg, 65% yield) was obtained as a brown oil. HPLC purity: 93%, MS (ESI+) m/z=600 [M+H]⁺. HPLC of mesylated intermediate: MS (ESI+) m/z=720 [M+H]⁺.

Step 5: To a solution of ethyl (2S)-2-[[(2S)-4-[5-[bis(2-chloroethyl)amino]-1-methyl-benzimidazol-2-yl]-2-(tert-butoxycarbonylamino)butanoyl]amino]-3-methyl-butanoate (93% purity, 216 mg, 0.334 mmol) in EtOH (2 mL) was added hydrogen chloride in dioxane (4.00 mol/L, 0.836 mL, 3.34 mmol) and stirred at 25° C. for 3h.

The crude was evaporated and purified by preparative chromatography (acidic buffer). The fractions obtained from the prep were kept in an ice bath until the LC-MS analysis had been performed. HCl in dioxane (4M, 1 mL) was added and the sample was immediately lyophilized to afford ethyl (2S)-2-[[(2S)-2-amino-4-[5-[bis(2-chloroethyl)amino]-1-methyl-benzimidazol-2-yl]butanoyl]amino]-3-methyl-butanoate; dihydrochloride (Example compound 3) (54 mg, 28% yield) as a slightly pale pink solid. HPLC purity: 98%, MS (ESI+) m/z=500 [M+H]⁺.

¹H NMR (600 MHz, DMSO-d6) δ 14.78 (s, 1H), 9.07 (d, J=7.3 Hz, 1H), 8.67-8.50 (m, 3H), 7.75 (d, J=9.1 Hz, 1H), 7.15 (d, J=9.2 Hz, 1H), 6.93 (d, J=2.4 Hz, 1H), 4.21 (dd, J=7.3, 5.5 Hz, 2H), 4.18-4.07 (m, 2H), 3.90 (s, 3H), 3.83 (t, J=6.7 Hz, 4H), 3.78 (t, J=6.2 Hz, 4H), 3.33 (dq, J=24.7, 8.5 Hz, 2H), 2.40-2.22 (m, 2H), 2.14 (ddt, J=14.0, 12.5, 6.9 Hz, 1H), 1.19 (t, J=7.1 Hz, 3H), 0.97 (dd, J=6.9, 5.4 Hz, 6H).

### Example 4: Ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4)

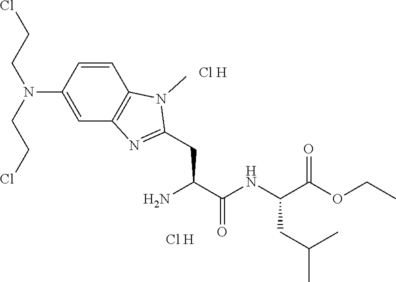

The title compound was prepared in 5 steps using the method described in Example 1.

Step 1: Ethyl (2S)-2-[[(2S)-2-(tert-butoxycarbonylamino)-3-(1-methyl-5-nitro-benzimidazol-2-yl)propanoyl]amino]-4-methyl-pentanoate (1.36 g, 2.42 mmol, 89% yield) was isolated as light yellow foam. HPLC purity: 90%, MS (ESI+) m/z=506 [M+H]⁺.

Step 2: The crude product, ethyl (2S)-2-[[(2S)-3-(5-amino-1-methyl-benzimidazol-2-yl)-2-(tert-butoxycarbonylamino)propanoyl]amino]-4-methyl-pentanoate (1.47 g, 2.78 mmol, 100% yield) was isolated as yellow oil and used directly in the next step without further purification. HPLC purity: 90%, MS (ESI+) m/z=476 [M+H]⁺.

Step 3: The crude product, ethyl (2S)-2-[[(2S)-3-[5-[bis(2-hydroxyethyl)amino]-1-methyl-benzimidazol-2-yl]-2-(tert-butoxycarbonylamino)propanoyl]amino]-4-methyl-pentanoate (1.50 g, 2.42 mmol, 86% yield) was obtained as brown solid. HPLC purity: 91%, MS (ESI+) m/z=564 [M+H]⁺.

Step 4: Ethyl (2S)-2-[[(2S)-3-[5-[bis(2-chloroethyl)amino]-1-methyl-benzimidazol-2-yl]-2-(tert-butoxycarbonylamino)propanoyl]amino]-4-methyl-pentanoate (0.550 g, 0.870 mmol, 33% yield) was isolated as a yellow oil. HPLC purity: 95%, MS (ESI+) m/z=600 [M+H]⁺. HPLC of mesylated intermediate: MS (ESI+) m/z=720 [M+H]⁺.

Step 5: To a solution of ethyl (2S)-2-[[(2S)-3-[5-[bis(2-chloroethyl)amino]-1-methyl-benzimidazol-2-yl]-2-(tert-butoxycarbonylamino)propanoyl]amino]-4-methyl-pentanoate (95%, 200 mg, 0.316 mmol) in EtOH (15 mL) was added hydrogen chloride in dioxane (4 M) (4.00 mol/L, 1.19 mL, 4.75 mmol), and the mixture was stirred at room temperature overnight. The crude was obtained by evaporation of the solvents, and purified by preparative chromatography affording ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4) (0.112 g, 0.189 mmol, 60% yield) as a white solid. HPLC purity: 100%, MS (ESI+) m/z=500 [M+H]⁺.

¹H NMR (600 MHz, DMSO-d6) δ 14.75 (br s, 1H), 9.29 (dd, J=7.8, 2.5 Hz, 1H), 8.86 (s, 3H), 7.78 (d, J=9.2 Hz, 1H), 7.18 (dd, J=9.4, 2.4 Hz, 1H), 6.91 (d, J=2.4 Hz, 1H), 4.49 (d, J=6.8 Hz, 1H), 4.34 (ddd, J=10.0, 7.5, 4.9 Hz, 1H), 4.06-4.01 (m, 2H), 3.99 (s, 3H), 3.90-3.74 (m, 9H), 3.65 (dd, J=15.4, 6.6 Hz, 1H), 1.72 (ddd, J=11.9, 7.9, 5.9 Hz, 1H), 1.56 (ddd, J=13.7, 10.0, 5.2 Hz, 1H), 1.50 (ddd, J=13.9, 9.0, 5.0 Hz, 1H), 1.15 (t, J=7.1 Hz, 3H), 0.88 (dd, J=16.2, 6.6 Hz, 6H).

### Example 5: Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate; dihydrochloride (Example compound 5)

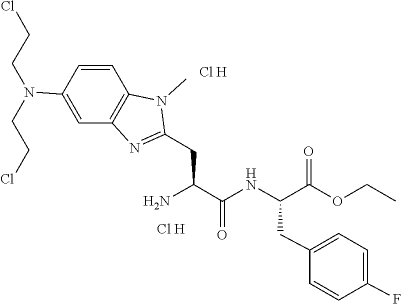

The title compound was prepared in 5 steps using the method described in Example 1.

Step 1: Ethyl (2S)-2-[[(2S)-2-(tert-butoxycarbonylamino)-3-(1-methyl-5-nitro-benzimidazol-2-yl)propanoyl]amino]-3-(4-fluorophenyl)propanoate (1.36 g, 2.19 mmol, 81% yield) was isolated as light yellow foam. HPLC purity: 90%, MS (ESI+) m/z=558 [M+H]⁺.

Step 2: The crude ethyl (2S)-2-[[(2S)-3-(5-amino-1-methyl-benzimidazol-2-yl)-2-(tert-butoxycarbonylamino)propanoyl]amino]-3-(4-fluorophenyl)propanoate (954 mg, 73% yield) was obtained as an off-white solid, which was used in the next step without further purification. HPLC purity: 98%, MS (ESI+) m/z=528 [M+H]⁺.

Step 3: The crude product ethyl (2S)-2-[[(2S)-3-[5-[bis(2-hydroxyethyl)amino]-1-methyl-benzimidazol-2-yl]-2-(tert-butoxycarbonylamino)propanoyl]amino]-3-(4-fluorophenyl)propanoate (964 mg, 85% yield) was obtained as a brown oil, which solidified upon standing. The compound was used in the next step without further purification. HPLC purity: 96%, MS (ESI+) m/z=616 [M+H]⁺.

Step 4: Ethyl (2S)-2-[[(2S)-3-[5-[bis(2-chloroethyl)amino]-1-methyl-benzimidazol-2-yl]-2-(tert-butoxycarbonylamino)propanoyl]amino]-3-(4-fluorophenyl)propanoate (0.635 g, 0.924 mmol, 69% yield) was isolated as a yellow oil. HPLC purity: 95%, MS (ESI+) m/z=652 [M+H]⁺. HPLC mesylated intermediate: MS (ESI+) m/z=772 [M+H]⁺.

Step 5: The crude product was purified by prep-HPLC (acidic conditions; flow 25 mL/min, gradient over 15 min from 12% B to 42% B. Fractions were collected based on the UV-signal at 239 nm). 4N HCl in dioxane (1 mL) was added to the combined fractions which were lyophilized to afford ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate; dihydrochloride (Example compound 5) (78 mg, 0.121 mmol, 13% yield) as an off-white solid. HPLC purity: 97%, MS (ESI+) m/z=552 [M+H]⁺.

¹H NMR (600 MHz, DMSO-d6) δ 14.66 (br s, 1H), 9.31 (d, J=7.5 Hz, 1H), 8.79 (s, 3H), 7.81-7.67 (m, 1H), 7.33-7.28 (m, 2H), 7.15 (d, J=9.2 Hz, 1H), 7.12-7.06 (m, 2H), 6.88 (d, J=2.4 Hz, 1H), 4.59 (p, J=8.4, 7.7 Hz, 1H), 4.41 (s, 1H), 4.01 (dddd, J=13.6, 10.8, 7.2, 3.8 Hz, 2H), 3.95 (s, 3H), 3.84 (ddt, J=23.1, 12.2, 6.2 Hz, 4H), 3.77 (ddd, J=9.3, 7.2, 4.8 Hz, 5H), 3.63 (s, 1H), 3.05-2.95 (m, 2H), 1.10 (t, J=7.1 Hz, 3H).

### Example 6: Ethyl (2S)-2-[[(2S)-2-amino-3-[5-[bis(2-chloroethyl)amino]-1-methyl-benzimidazol-2-yl]propanoyl]amino]-3-methyl-butanoate; dihydrochloride (Example compound 6)

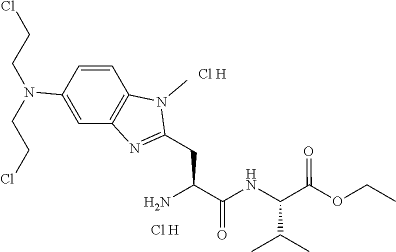

The title compound was prepared in 5 steps using the method described in Example 1.

Step 1: The desired product, ethyl (2S)-2-[[(2S)-2-(tert-butoxycarbonylamino)-3-(1-methyl-5-nitro-benzimidazol-2-yl)propanoyl]amino]-3-methyl-butanoate (0.992 g, 1.82 mmol, 67% yield), was isolated as a light yellow foam. HPLC purity: 90%, MS (ESI+) m/z=492 [M+H]⁺.

Step 2: The crude product, ethyl (2S)-2-[[(2S)-3-(5-amino-1-methyl-benzimidazol-2-yl)-2-(tert-butoxycarbonylamino)propanoyl]amino]-3-methyl-butanoate (931 mg, 98% yield), was obtained as a brown oil, which was used in the next step without further purification. HPLC purity: 98%, MS (ESI+) m/z=462 [M+H]⁺.

Step 3: The crude product, ethyl (2S)-2-[[(2S)-3-[5-[bis(2-hydroxyethyl)amino]-1-methyl-benzimidazol-2-yl]-2-(tert-butoxycarbonylamino)propanoyl]amino]-3-methyl-butanoate (0.848 g, 1.39 mmol, 70% yield), was obtained as a yellow oil and used in the next step without further purification. HPLC purity: 90%, MS (ESI+) m/z=550 [M+H]⁺

Step 4: Ethyl (2S)-2-[[(2S)-3-[5-[bis(2-chloroethyl)amino]-1-methyl-benzimidazol-2-yl]-2-(tert-butoxycarbonylamino)propanoyl]amino]-3-methyl-butanoate (0.210 g, 0.340 mmol, yield: 22%) was isolated as a yellow oil. HPLC purity: 95%, MS (ESI+) m/z=586 [M+H]⁺. HPLC mesylated intermediate: MS (ESI+) m/z=706 [M+H]⁺.

Step 5: The crude product was purified by preparative HPLC (flow 25 mL/min, gradient over 15 min from 8% B to 38% B; fractions were collected based on the UV-signal at 239 nm) to afford ethyl (2S)-2-[[(2S)-2-amino-3-[5-[bis(2-chloroethyl)amino]-1-methyl-benzimidazol-2-yl]propanoyl]amino]-3-methyl-butanoate; dihydrochloride (Example compound 6) (85 mg, 0.147 mmol, 43% yield) as an off-white solid. HPLC purity: 97%, MS (ESI+) m/z=486 [M+H]⁺.

¹H NMR (600 MHz, DMSO-d6) δ 14.77 (br s, 1H), 9.04 (d, J=7.9 Hz, 1H), 8.85 (s, 3H), 7.78 (d, J=9.2 Hz, 1H), 7.17 (dd, J=9.6, 2.3 Hz, 1H), 6.90 (d, J=2.4 Hz, 1H), 4.59 (s, 1H), 4.26 (dd, J=7.9, 5.5 Hz, 1H), 4.07-3.99 (m, 2H), 3.98 (s, 3H), 3.89-3.75 (m, 9H), 3.59 (dd, J=15.5, 6.8 Hz, 1H), 2.12-2.03 (m, 1H), 1.15 (t, J=7.1 Hz, 3H), 0.92 (d, J=6.8 Hz, 6H).

### Example 7: Ethyl (2S)-2-[[(2S)-2-amino-4-methyl-pentanoyl]amino]-4-[5-[bis(2-chloroethyl)amino]-1-methyl-benzimidazol-2-yl]butanoate; dihydrochloride (Example compound 7)

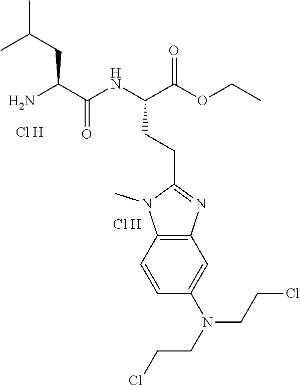

Step 1: To a solution of ethyl (2S)-2-amino-4-(1-methyl-5-nitro-benzimidazol-2-yl)butanoate (Intermediate 1) (1.00 g, 3.26 mmol), (2S)-2-(tert-butoxycarbonylamino)-4-methyl-pentanoic acid; hydrate (0.814 g, 3.26 mmol) and 1-methylimidazole (0.546 mL, 6.86 mmol) in MeCN (30 mL) was added [chloro(dimethylamino)methylene]-dimethyl-ammonium; hexafluorophosphate (1.01 g, 3.59 mmol) at room temperature. The mixture was stirred at room temperature for 1 h. The reaction was quenched with citric acid solution (10% in water) and the volatile were evaporated. The crude was diluted with water and extracted with EtOAc (×3). The organic phase was dried over MgSO₄, filtered and evaporated under reduced pressure. The crude was purified by silica chromatography using DCM->DCM/MeOH (10%) affording ethyl (2S)-2-[[(2S)-2-(tert-butoxycarbonylamino)-4-methyl-pentanoyl]amino]-4-(1-methyl-5-nitro-benzimidazol-2-yl)butanoate (90%, 1.47 g, 2.54 mmol, 78% yield) as a light yellow foam. HPLC purity: 90%; MS (ESI+) m/z=520 [M+H]⁺.

Step 2: Ethyl (2S)-2-[[(2S)-2-(tert-butoxycarbonylamino)-4-methyl-pentanoyl]amino]-4-(1-methyl-5-nitro-benzimidazol-2-yl)butanoate (1.47 g, 0.00283 mol) was dissolved in EtOH (30 mL) and placed in a H-reactor. Pd/C (10%, 0.151 g, 0.000141 mol) was added and the reactor was sealed. A nitrogen purge was conducted. H₂ was charged to the reactor at 3 bar and the reaction was stirred at 35° C. for 1.5 h. Hydrogen was vented with a nitrogen purge. Catalyst was removed by filtration through Celite. The solvent was evaporated and the crude product, ethyl (2S)-4-(5-amino-1-methyl-benzimidazol-2-yl)-2-[[(2S)-2-(tert-butoxycarbonylamino)-4-methyl-pentanoyl]amino]butanoate (1.20 g, 2.21 mmol, 78% yield), was obtained as a brown foam, which was used in the next step without further purification. HPLC purity: 90%; MS (ESI+) m/z=490 [M+H]⁺.

Step 3: Ethyl (2S)-4-(5-amino-1-methyl-benzimidazol-2-yl)-2-[[(2S)-2-(tert-butoxycarbonylamino)-4-methyl-pentanoyl]amino]butanoate (1.26 g, 2.45 mmol, 95% pure) was dissolved in acetic acid (5 mL). Oxirane (2.3M, 10.6 mL) was added and the reaction was stirred at room temperature for 24 hours. The volatiles were removed under vacuum, the crude material was dissolved in DCM, and washed with saturated aqueous NaHCO₃. The organic layer was washed with water and brine, passed through a phase separator cartridge and concentrated to afford ethyl (2S)-4-[5-[bis(2-hydroxyethyl)amino]-1-methyl-benzimidazol-2-yl]-2-[[(2S)-2-(tert-butoxycarbonylamino)-4-methyl-pentanoyl]amino]butanoate (1.38 g, 93% yield) as a brown oil, which solidified upon standing. The product was used in the next step without further purification. HPLC purity: 95%; MS (ESI+) m/z=578 [M+H]⁺.

Step 4: To a solution of ethyl (2S)-4-[5-[bis(2-hydroxyethyl)amino]-1-methyl-benzimidazol-2-yl]-2-[[(2S)-2-(tert-butoxycarbonylamino)-4-methyl-pentanoyl]amino]butanoate (1.38 g, 2.27 mmol, 95% purity) and triethylamine (1.27 mL, 9.08 mmol) in DCM (50 mL) was added methanesulfonyl chloride (0.544 mL, 7.03 mmol) and the mixture was stirred at room temperature for 10 min. The reaction mixture was washed with a mixture of NaHCO₃(sat):water (2:5) (100 mL), 0.3 M HCl (100 mL) and brine (100 mL). The organic phase was passed through a phase separator cartridge and evaporated under reduced pressure. The crude ethyl (2S)-4-[5-[bis(2-methylsulfonyloxyethyl)amino]-1-methyl-benzimidazol-2-yl]-2-[[(2S)-2-(tert-butoxycarbonylamino)-4-methyl-pentanoyl]amino]butanoate was immediately used in the next step without further purification.

Ethyl (2S)-4-[5-[bis(2-methylsulfonyloxyethyl)amino]-1-methyl-benzimidazol-2-yl]-2-[[(2S)-2-(tert-butoxycarbonylamino)-4-methyl-pentanoyl]amino]butanoate (1.82 g, 1.64 mmol, 66% purity) was dissolved in DMF (5 mL). LiCl (1.04 g, 24.6 mmol) was added and the reaction was heated at 60° C. for 1h. The reaction mixture was diluted with a mixture of toluene (10 mL) and EtOAc (10 mL), then washed with brine (3×20 mL), dried over MgSO₄, filtered and evaporated. The crude obtained was purified by flash chromatography using a gradient of ethyl acetate in DCM from 0-20%. The fractions containing the desired compound were combined and concentrated to afford ethyl (2S)-4-[5-[bis(2-chloroethyl)amino]-1-methyl-benzimidazol-2-yl]-2-[[(2S)-2-(tert-butoxycarbonylamino)-4-methyl-pentanoyl]amino]butanoate (652 mg, 62% yield) as a brown oil. HPLC of mesylated intermediate: MS (ESI+) m/z=734 [M+H]⁺. HPLC purity of desired product: 96%; MS (ESI+) m/z=614 [M+H]⁺.

Step 5: To a solution of ethyl (2S)-4-[5-[bis(2-chloroethyl)amino]-1-methyl-benzimidazol-2-yl]-2-[[(2S)-2-(tert-butoxycarbonylamino)-4-methyl-pentanoyl]amino]butanoate (205 mg, 0.316 mmol, 95% purity) in EtOH (2 mL) was added hydrogen chloride in dioxane (4.00 mol/L, 1.19 mL, 4.75 mmol) and the mixture was stirred at 25° C. for 3h. The crude was obtained by evaporation of the solvents, and purified by preparative chromatography (acidic buffer, flow 25 mL/min, gradient over 15 min from 7% B to 37% B. Fractions were collected based on the UV-signal at 235 nm). The fractions were kept in an ice bath until the LC-MS analysis had been performed. HCl in dioxane (4M, 1 mL) was added and the sample was immediately lyophilized to afford ethyl (2S)-2-[[(2S)-2-amino-4-methyl-pentanoyl]amino]-4-[5-[bis(2-chloroethyl)amino]-1-methyl-benzimidazol-2-yl]butanoate; dihydrochloride (Example compound 7) (50 mg, 26% yield) as a yellow oil. HPLC purity: 95%, MS (ESI+) m/z=514 [M+H]⁺.

¹H NMR (600 MHz, DMSO-d6) δ 14.41 (s, 1H), 9.04 (d, J=7.9 Hz, 1H), 8.22 (d, J=5.3 Hz, 3H), 7.74 (d, J=9.2 Hz, 1H), 7.15 (dd, J=9.4, 2.3 Hz, 1H), 6.90 (d, J=2.4 Hz, 1H), 4.51 (ddd, J=9.6, 8.0, 4.9 Hz, 2H), 4.10 (d, J=7.1 Hz, 2H), 3.88 (s, 3H), 3.84 (t, J=6.7 Hz, 4H), 3.78 (t, J=6.9 Hz, 4H), 3.18 (d, J=8.6 Hz, 2H), 2.42-2.32 (m, 1H), 2.19-2.09 (m, 1H), 1.70 (dp, J=13.4, 6.8 Hz, 1H), 1.61 (dt, J=13.9, 7.0 Hz, 1H), 1.55 (dt, J=14.1, 7.5 Hz, 1H), 1.19 (t, J=7.1 Hz, 3H), 0.92 (dd, J=16.4, 6.5 Hz, 6H).

### Example 8: Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate; dihydrochloride (Example compound 8)

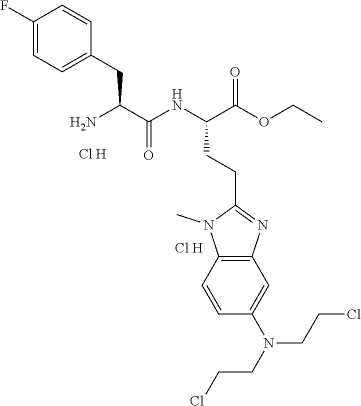

The title compound was prepared in 5 steps using the method described in Example 7.

Step 1: Ethyl (2S)-2-[[(2S)-2-(tert-butoxycarbonylamino)-3-(4-fluorophenyl)propanoyl]amino]-4-(1-methyl-5-nitro-benzimidazol-2-yl)butanoate (1.87 g, 2.94 mmol, 90% yield) was isolated as a light yellow foam. HPLC purity: 90%, MS (ESI+) m/z=572 [M+H]⁺.

Step 2: The crude ethyl (2S)-4-(5-amino-1-methyl-benzimidazol-2-yl)-2-[[(2S)-2-(tert-butoxycarbonylamino)-3-(4-fluorophenyl)propanoyl]amino]butanoate (1.78 g, 90% yield) was obtained as a light yellow solid, which was used in the next step without further purification. HPLC purity: 90%, MS (ESI+) m/z=542 [M+H]⁺.

Step 3: The crude product, ethyl (2S)-4-[5-[bis(2-hydroxyethyl)amino]-1-methyl-benzimidazol-2-yl]-2-[[(2S)-2-(tert-butoxycarbonylamino)-3-(4-fluorophenyl)propanoyl]amino]butanoate (1.74 g, 86% yield), was obtained as a light brown solid. The compound was used in the next step without further purification. HPLC purity: 92%, MS (ESI+) m/z=630 [M+H]⁺.

Step 4: Ethyl (2S)-4-[5-[bis(2-chloroethyl)amino]-1-methyl-benzimidazol-2-yl]-2-[[(2S)-2-(tert-butoxycarbonylamino)-3-(4-fluorophenyl)propanoyl]amino]butanoate (628 mg, 37% yield) was isolated as a brown oil. HPLC purity: 92%, MS (ESI+) m/z=666 [M+H]⁺. HPLC mesylated intermediate: MS (ESI+) m/z=786 [M+H]⁺.

Step 5: The crude product was purified by preparative HPLC (acidic conditions; flow 25 mL/min, gradient over 15 min from 11% B to 40% B. Fractions were collected based on the UV-signal at 235 nm). HCl in dioxane (4M, 1 mL) was added to the combined fractions and the sample was immediately lyophilized to afford ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate; dihydrochloride (Example compound 8) (35 mg, 23% yield) as an off-white solid. HPLC purity: 95%, MS (ESI+) m/z=566 [M+H]⁺.

¹H NMR (600 MHz, DMSO-d6) δ 14.61 (s, 1H), 9.57 (d, J=7.6 Hz, 1H), 8.40 (d, J=5.5 Hz, 3H), 7.75 (d, J=9.2 Hz, 1H), 7.45-7.36 (m, 2H), 7.15 (dtd, J=9.4, 7.4, 6.9, 2.3 Hz, 3H), 6.91 (d, J=2.4 Hz, 1H), 4.44 (ddd, J=9.5, 7.6, 4.3 Hz, 1H), 4.22 (d, J=6.8 Hz, 1H), 4.08 (q, J=7.1 Hz, 2H), 3.95 (s, 3H), 3.83 (t, J=6.8 Hz, 4H), 3.78 (t, J=6.1 Hz, 4H), 3.36-3.25 (m, 2H), 3.19 (dd, J=14.0, 5.7 Hz, 1H), 2.98 (dd, J=14.0, 8.0 Hz, 1H), 2.40-2.31 (m, 1H), 2.17 (dtd, J=14.6, 9.5, 5.5 Hz, 1H), 1.18 (t, J=7.1 Hz, 3H).

### Example 9: Ethyl (2S)-2-[[(2S)-2-amino-3-methyl-butanoyl]amino]-4-[5-[bis(2-chloroethyl)amino]-1-methyl-benzimidazol-2-yl]butanoate; dihydrochloride (Example compound 9)

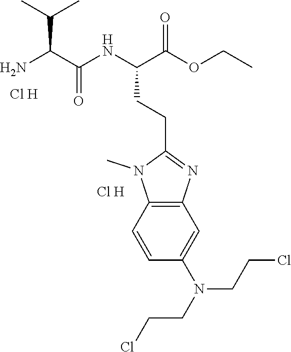

The title compound was prepared in 5 steps using the method described in Example 7.

Step 1: Ethyl (2S)-2-[[(2S)-2-(tert-butoxycarbonylamino)-3-methyl-butanoyl]amino]-4-(1-methyl-5-nitro-benzimidazol-2-yl)butanoate (0.757 g, 1.35 mmol, 41% yield) was isolated as light yellow foam. HPLC purity: 90%, MS (ESI+) m/z=506 [M+H]⁺.

Step 2: The crude product, ethyl (2S)-4-(5-amino-1-methyl-benzimidazol-2-yl)-2-[[(2S)-2-(tert-butoxycarbonylamino)-3-methyl-butanoyl]amino]butanoate (0.642 g, 1.28 mmol, yield: 86%) was obtained as a light brown solid, which was used in the next step without further purification. HPLC purity: 95%, MS (ESI+) m/z=476 [M+H]⁺.

Step 3: The crude product, ethyl (2S)-4-[5-[bis(2-hydroxyethyl)amino]-1-methyl-benzimidazol-2-yl]-2-[[(2S)-2-(tert-butoxycarbonylamino)-3-methyl-butanoyl]amino]butanoate (0.685 g, 1.09 mmol, 85% yield) was obtained as a brown oil, which solidified upon standing. The compound was used in the next step without further purification. HPLC purity: 90%, MS (ESI+) m/z=564 [M+H]⁺.

Step 4: Ethyl (2S)-4-[5-[bis(2-chloroethyl)amino]-1-methyl-benzimidazol-2-yl]-2-[[(2S)-2-(tert-butoxycarbonylamino)-3-methyl-butanoyl]amino]butanoate (0.402 g, 0.623 mmol, 58% yield) was isolated as a light brown oil. HPLC purity: 93%, MS (ESI+) m/z=600 [M+H]⁺. HPLC mesylated intermediate: MS (ESI+) m/z=720 [M+H]⁺.

Step 5: The crude product was evaporated and purified by preparative chromatography (acidic buffer; flow 25 mL/min, gradient over 15 min from 7% B to 36% B. Fractions were collected based on the UV-signal at 235 nm). The fractions obtained from the prep were kept in an ice bath, HCl in dioxane (4M, 1 mL) was added and the sample was immediately lyophilized to afford ethyl (2S)-2-[[(2S)-2-amino-3-methyl-butanoyl]amino]-4-[5-[bis(2-chloroethyl)amino]-1-methyl-benzimidazol-2-yl]butanoate; dihydrochloride (Example compound 9) (12 mg, 6.3% yield) as an off-white solid. HPLC purity: 95%, MS (ESI+) m/z=500 [M+H]⁺.

¹H NMR (600 MHz, DMSO-d6) δ 14.70 (s, 1H), 9.37 (d, J=7.6 Hz, 1H), 8.44 (d, J=5.7 Hz, 3H), 7.75 (d, J=9.2 Hz, 1H), 7.15 (dd, J=9.3, 2.4 Hz, 1H), 6.91 (d, J=2.4 Hz, 1H), 4.46 (ddd, J=10.0, 7.6, 4.0 Hz, 1H), 4.10 (d, J=7.1 Hz, 2H), 3.96 (s, 3H), 3.83 (t, J=6.6 Hz, 4H), 3.80-3.77 (m, 4H), 3.74-3.63 (m, 1H), 3.52-3.41 (m, 1H), 3.31 (ddd, J=15.9, 9.6, 6.8 Hz, 1H), 2.37 (dt, J=13.3, 4.9 Hz, 1H), 2.20-2.10 (m, 2H), 1.18 (t, J=7.1 Hz, 3H), 1.04-0.94 (m, 6H).

### Example 10: Ethyl (2S)-2-[[(2S)-2-amino-4-methyl-pentanoyl]amino]-3-[5-[bis(2-chloroethyl)amino]-1-methyl-benzimidazol-2-yl]propanoate; dihydrochloride (Example compound 10)

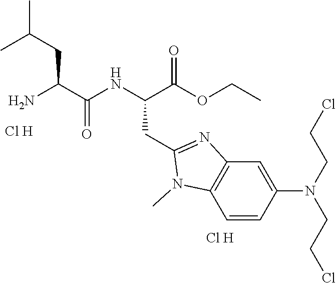

The title compound was prepared in 5 steps using the method described in Example 7.

Step 1: Ethyl (2S)-2-[[(2S)-2-(tert-butoxycarbonylamino)-4-methyl-pentanoyl]amino]-3-(1-methyl-5-nitro-benzimidazol-2-yl)propanoate (1.68 g, 2.99 mmol, 87% yield) was isolated as a light yellow foam. HPLC purity: 90%, MS (ESI+) m/z=506 [M+H]⁺.

Step 2: The crude product, ethyl (2S)-3-(5-amino-1-methyl-benzimidazol-2-yl)-2-[[(2S)-2-(tert-butoxycarbonylamino)-4-methyl-pentanoyl]amino]propanoate (1.50 g, 2.90 mmol, 87% yield), was obtained as a brown foam, which was used in the next step without further purification. HPLC purity: 92%, MS (ESI+) m/z=476 [M+H]⁺.

Step 3: The crude product, ethyl (2S)-3-[5-[bis(2-hydroxyethyl)amino]-1-methyl-benzimidazol-2-yl]-2-[[(2S)-2-(tert-butoxycarbonylamino)-4-methyl-pentanoyl]amino]propanoate (1.20 g, 1.60 mmol, 52% yield) was obtained as a brown solid. HPLC purity: 75%, MS (ESI+) m/z=564 [M+H]⁺.

Step 4: Ethyl (2S)-3-[5-[bis(2-chloroethyl)amino]-1-methyl-benzimidazol-2-yl]-2-[[(2S)-2-(tert-butoxycarbonylamino)-4-methyl-pentanoyl]amino]propanoate (0.430 g, 0.680 mmol, 32% yield) was isolated as a yellow oil. HPLC purity: 95%, MS (ESI+) m/z=600 [M+H]⁺. HPLC mesylated intermediate: MS (ESI+) m/z=720 [M+H]⁺.

Step 5: The crude product was purified by prep-HPLC (acidic conditions; flow 25 mL/min, gradient over 15 min from 6% B to 36% B. Fractions were collected based on the UV-signal at 237 nm). 4N HCl in dioxane (1 mL) was added to the combined fractions which were lyophilized to afford ethyl (2S)-2-[[(2S)-2-amino-4-methyl-pentanoyl]amino]-3-[5-[bis(2-chloroethyl)amino]-1-methyl-benzimidazol-2-yl]propanoate; dihydrochloride (Example compound 10) (0.102 g, 0.169 mmol, 53% yield) as a white solid. HPLC purity: 95%, MS (ESI+) m/z=500 [M+H]⁺.

¹H NMR (600 MHz, DMSO-d6) δ 14.85 (br s, 1H), 9.56 (d, J=7.7 Hz, 1H), 8.40 (s, 3H), 7.76 (d, J=9.2 Hz, 1H), 7.16 (d, J=9.2 Hz, 1H), 6.95 (d, J=2.5 Hz, 1H), 5.02 (q, J=7.6 Hz, 1H), 4.12 (q, J=7.1 Hz, 2H), 3.97 (s, 3H), 3.87-3.73 (m, 9H), 3.67 (dd, J=15.8, 6.1 Hz, 1H), 3.58 (dd, J=15.6, 8.2 Hz, 1H), 1.64 (dt, J=13.4, 6.7 Hz, 1H), 1.52 (hept, J=6.8 Hz, 2H), 1.14 (t, J=7.1 Hz, 3H), 0.87 (dd, J=9.5, 6.5 Hz, 6H).

### Example 11: Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate; dihydrochloride (Example compound 11)

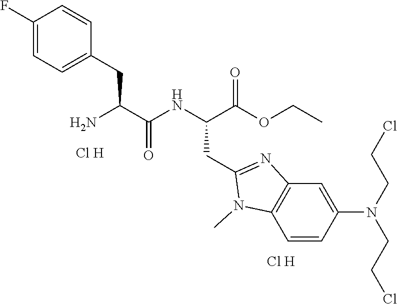

The title compound was prepared in 5 steps using the method described in Example 7.

Step 1: Ethyl (2S)-2-[[(2S)-2-(tert-butoxycarbonylamino)-3-(4-fluorophenyl)propanoyl]amino]-3-(1-methyl-5-nitro-benzimidazol-2-yl)propanoate (0.794 g, 1.14 mmol, 29% yield) was isolated as a light yellow solid. HPLC purity: 80%, MS (ESI+) m/z=558 [M+H]⁺.

Step 2: The crude ethyl (2S)-3-(5-amino-1-methyl-benzimidazol-2-yl)-2-[[(2S)-2-(tert-butoxycarbonylamino)-3-(4-fluorophenyl)propanoyl]amino]propanoate (772 mg, 93% yield) was obtained as a light brown solid, which was used in the next step without further purification. HPLC purity: 91%, MS (ESI+) m/z=528 [M+H]⁺.

Step 3: The crude product, ethyl (2S)-3-[5-[bis(2-hydroxyethyl)amino]-1-methyl-benzimidazol-2-yl]-2-[[(2S)-2-(tert-butoxycarbonylamino)-3-(4-fluorophenyl)propanoyl]amino]propanoate (817 mg, 91% yield) was obtained as a brown oil, which solidified upon standing. The compound was used in the next step without further purification. HPLC purity: 95%, MS (ESI+) m/z=616 [M+H]⁺.

Step 4: Ethyl (2S)-3-[5-[bis(2-chloroethyl)amino]-1-methyl-benzimidazol-2-yl]-2-[[(2S)-2-(tert-butoxycarbonylamino)-3-(4-fluorophenyl)propanoyl]amino]propanoate (449 mg, 51% yield) was isolated as a brown oil. HPLC purity: 86%, MS (ESI+) m/z=652 [M+H]⁺ HPLC mesylated intermediate: MS (ESI+) m/z=772 [M+H]⁺.

Step 5: The crude product was purified by preparative chromatography (acidic buffer; flow 25 mL/min, gradient over 15 min from 10% B to 40% B. Fractions were collected based on the UV-signal at 237 nm). HCl in dioxane (4M, 1 mL) was added to the combined fractions and the sample was immediately lyophilized to afford ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate; dihydrochloride (Example compound 11) (12 mg, 15% yield) as an off-white solid. HPLC purity: 96%, MS (ESI+) m/z=552 [M+H]⁺.

¹H NMR (600 MHz, DMSO-d6) δ 14.73 (s, 1H), 9.64 (s, 1H), 8.32 (s, 3H), 7.77-7.67 (m, 1H), 7.33 (dt, J=9.6, 4.9 Hz, 2H), 7.14 (t, J=8.7 Hz, 3H), 6.94 (d, J=2.4 Hz, 1H), 4.98 (q, J=7.5 Hz, 1H), 4.12 (dtt, J=10.8, 6.7, 3.7 Hz, 3H), 3.96 (s, 3H), 3.83 (t, J=6.8 Hz, 4H), 3.77 (t, J=6.4 Hz, 4H), 3.14 (dd, J=14.1, 6.0 Hz, 1H), 2.97 (dd, J=14.1, 7.8 Hz, 1H), 1.13 (t, J=7.1 Hz, 3H), 0.87-0.81 (m, 2H).

### Example 12: Ethyl (2S)-2-[[(2S)-2-amino-3-methyl-butanoyl]amino]-3-[5-[bis(2-chloroethyl)amino]-1-methyl-benzimidazol-2-yl]propanoate; dihydrochloride (Example compound 12)

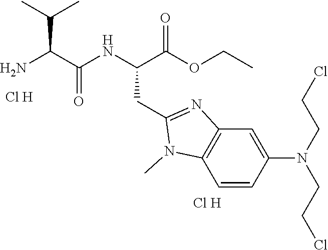

The title compound was prepared in 5 steps using the method described in Example 7.

Step 1: Ethyl (2S)-2-[[(2S)-2-(tert-butoxycarbonylamino)-3-methyl-butanoyl]amino]-3-(1-methyl-5-nitro-benzimidazol-2-yl)propanoate (0.428 g, 0.784 mmol, 23% yield) was isolated as a yellow solid. HPLC purity: 90%, MS (ESI+) m/z=492 [M+H]⁺.

Step 2: The crude ethyl (2S)-3-(5-amino-1-methyl-benzimidazol-2-yl)-2-[[(2S)-2-(tert-butoxycarbonylamino)-3-methyl-butanoyl]amino]propanoate (393 mg, 93% yield) was obtained as a yellow solid, which was used in the next step without further purification. HPLC purity: 95%, MS (ESI+) m/z=462 [M+H]⁺.

Step 3: The crude product, ethyl (2S)-3-[5-[bis(2-hydroxyethyl)amino]-1-methyl-benzimidazol-2-yl]-2-[[(2S)-2-(tert-butoxycarbonylamino)-3-methyl-butanoyl]amino]propanoate (0.305 g, 0.499 mmol, 58% yield) was obtained as a brown solid. The compound was used in the next step without further purification. HPLC purity: 90%, MS (ESI+) m/z=550 [M+H]⁺.

Step 4: Ethyl (2S)-3-[5-[bis(2-chloroethyl)amino]-1-methyl-benzimidazol-2-yl]-2-[[(2S)-2-(tert-butoxycarbonylamino)-3-methyl-butanoyl]amino]propanoate (0.168 g, 0.272 mmol, 37% yield) was isolated as a yellow oil. HPLC purity: 95%, MS (ESI+) m/z=586 [M+H]⁺ HPLC mesylated intermediate: MS (ESI+) m/z=706 [M+H]⁺.

Step 5: The crude product was evaporated and purified by preparative HPLC (acidic conditions; flow 25 mL/min, gradient over 15 min from 5% B to 34% B. Fractions were collected based on the UV-signal at 238 nm). HCl in dioxane (4M, 1 mL) was added to the combined fractions and the solution was immediately lyophilized to afford ethyl (2S)-2-[[(2S)-2-amino-3-methyl-butanoyl]amino]-3-[5-[bis(2-chloroethyl)amino]-1-methyl-benzimidazol-2-yl]propanoate; dihydrochloride (Example compound 12) (45 mg, 0.0724 mmol, 27% yield). HPLC purity: 90%, MS (ESI+) m/z=486 [M+H]⁺.

¹H NMR (600 MHz, DMSO-d6) δ 13.82 (br s, 1H), 8.51 (s, 1H), 7.49-7.30 (m, 3H), 6.85 (d, J=9.2 Hz, 1H), 6.25 (d, J=9.3 Hz, 1H), 6.05 (d, J=2.5 Hz, 1H), 4.15 (q, J=7.4 Hz, 1H), 3.22 (q, J=7.1 Hz, 2H), 3.06 (s, 3H), 2.96-2.86 (m, 8H), 2.80 (dt, J=9.3, 4.1 Hz, 2H), 2.66 (d, J=8.2 Hz, 1H), 1.22 (dq, J=13.4, 6.7 Hz, 1H), 0.23 (t, J=7.1 Hz, 3H), 0.11-0.00 (m, 6H).

### Example 13: Ethyl (2S)-2-[[(2S)-2-amino-4-[5-[bis(2-chloroethyl)amino]-1-phenyl-benzimidazol-2-yl]butanoyl]amino]-4-methyl-pentanoate; hydrochloride (Example compound 13)

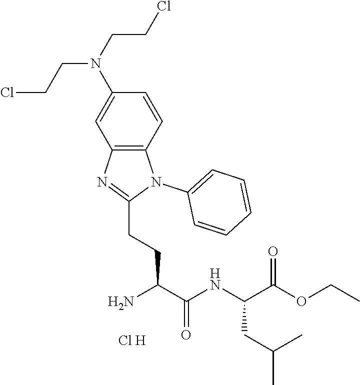

Example 13 was synthesized according to Scheme 4 below:

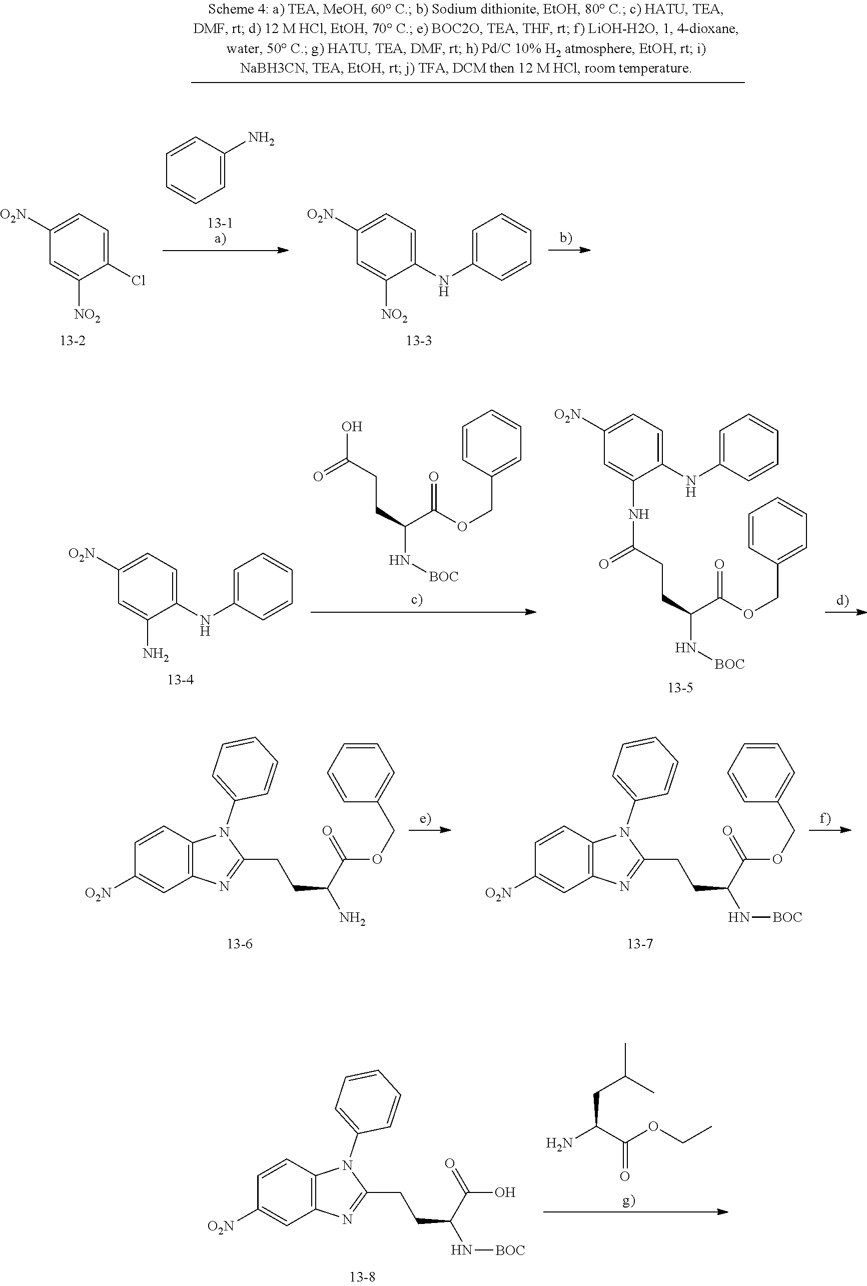

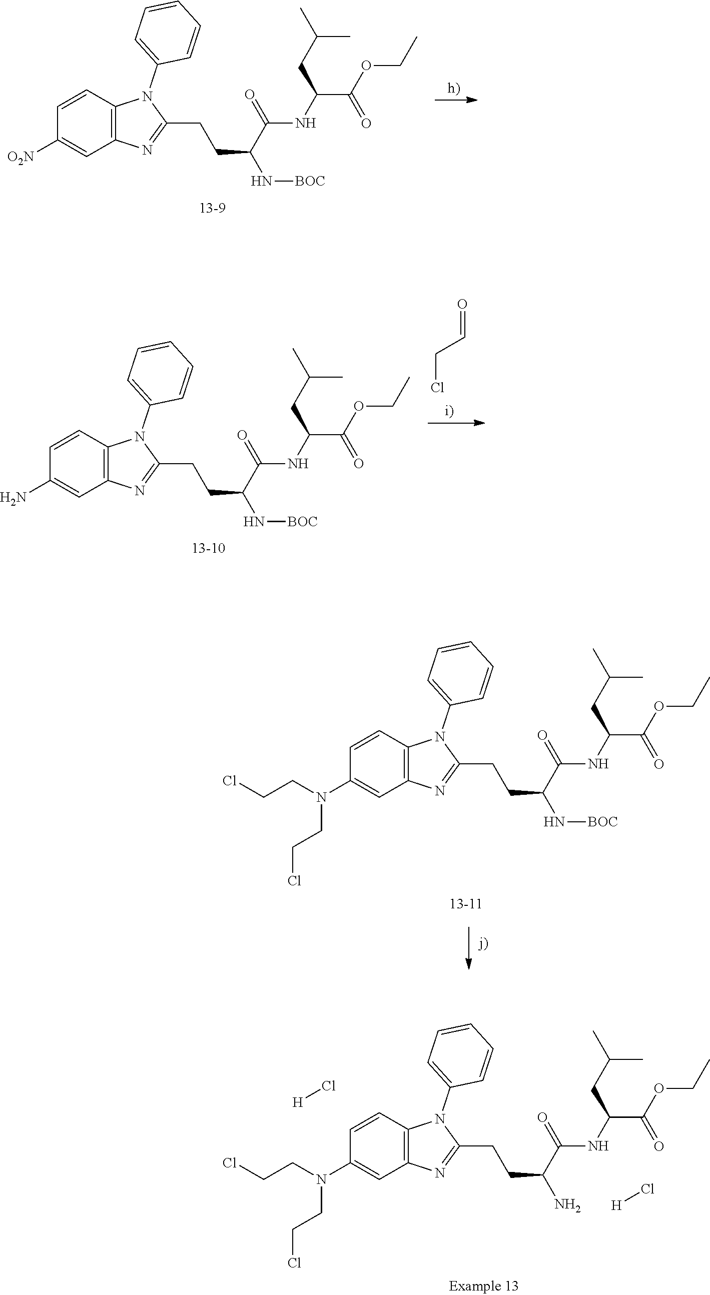

### 2,4-Dinitro-N-phenyl-aniline (compound 13-3)

TEA (6.19 mL, 44.4 mmol) was added to a solution of 1-chloro-2,4-dinitro-benzene (3.00 g, 14.8 mmol, compound 13-2) and aniline (1.35 mL, 14.8 mmol, compound 13-1) in MeOH (150 mL). The reaction was stirred at 60° C. overnight. The reaction was removed from heating and a light brick red solid was formed. The solid was filtered of and washed by ice cold MeOH to give the title compound (3.26 g, 12.54 mmol, 85% yield). HPLC purity: 95%, MS (ESI+) m/z=260 [M+H]⁺.

### 4-Nitro-N1-phenyl-benzene-1,2-diamine (compound 13-4)

Sodium dithionite (6.57 g, 37.7 mmol) was added to 2,4-dinitro-N-phenyl-aniline (3.26 g, 12.6 mmol) in a solvent mixture of EtOH (120 mL) and water (30 mL) at 80° C. The reaction was stirred for 1h. The organic solvent was evaporated, and more water was added. The compound was extracted with DCM. The water phase was discarded. DCM phase contains solid which was filtered of. The filtrate was washed by brine, dried over MgSO₄, filtered and the solvent was evaporated to give the title compound (1.25 g, 5.45 mmol, 43% yield). HPLC purity: 95%, MS (ESI+) m/z=230 [M+H]⁺.

### Benzyl (2S)-5-(2-anilino-5-nitro-anilino)-2-(tert-butoxycarbonylamino)-5-oxo-pentanoate (compound 13-5)

HATU (2.28 g, 6.00 mmol) was added to (4S)-5-benzyloxy-4-(tert-butoxycarbonylamino)-5-oxo-pentanoic acid (1.93 g, 5.73 mmol) and TEA (2.28 mL, 16.4 mmol) in DMF (15 mL). To this mixture was added 4-nitro-N1-phenyl-benzene-1,2-diamine (1.25 g, 5.45 mmol). The reaction was stirred overnight. Toluene (70 mL) was added and the organic phase was washed by water (100 mL) and brine (60 mL). The organic phase was dried over MgSO₄, filtered and the solvent was evaporated to give the title compound (2.90 g, 5.45 mmol, 97% yield). HPLC purity: 88%, MS (ESI+) m/z=449 [M+H-BOC]+. The material was used as such in next step.

### Benzyl (2S)-2-amino-4-(5-nitro-1-phenyl-benzimidazol-2-yl)butanoate (compound 13-6)

To a solution of benzyl (2S)-5-(2-anilino-5-nitro-anilino)-2-(tert-butoxycarbonylamino)-5-oxo-pentanoate (2.99 g, 5.45 mmol) in EtOH (37 mL) was added 12 M HCl (8.18 mL, 98.1 mmol) and the mixture was stirred at 70° C. for 40 minutes. The crude was diluted with water (200 mL) and carefully neutralized to pH 7 with 4 M NaOH. The product was extracted with ethyl acetate (3×100 mL) and the organic phase was dried over MgSO₄, filtered and the solvent was evaporated to give the title compound (2.35 g, 5.45 mol, quantitative yield). HPLC purity: 65%, MS (ESI+) m/z=431 [M+H]⁺. The material was used as such in next step.

### Benzyl (2S)-2-(tert-butoxycarbonylamino)-4-(5-nitro-1-phenyl-benzimidazol-2-yl)butanoate (compound 13-7)

Benzyl (2S)-2-amino-4-(6-nitro-3-phenyl-imidazo[4,5-b]pyridin-2-yl)butanoate (2.35 g, 0.00545 mol) in THF (25 mL) was added TEA (2.28 mL, 0.0163 mol) and tert-butoxycarbonyl tert-butyl carbonate (1.43 g, 6.54 mmol). The mixture was stirred at room temperature over weekend. To remove excess BOC₂O imidazole (0.111 g, 1.63 mol) was added and the reaction was further stirred for 1 h. The solvent was evaporated, and the residue was dissolved in ethyl acetate. The organic phase was washed by 5% citric acid, dried over MgSO₄, filtered and the solvent was evaporated. The crude was flash chromatographed on silica gradient eluting with 20 to 30% ethyl acetate in p-ether to give the title compound (2.21 g, 4.17 mmol, 76% yield). HPLC purity: 77%, MS (ESI+) m/z=531 [M+H]⁺.

### (2S)-2-(tert-Butoxycarbonylamino)-4-(5-nitro-1-phenyl-benzimidazol-2-yl)butanoic acid (compound 13-8)

To a solution of benzyl (2S)-2-(tert-butoxycarbonylamino)-4-(5-nitro-1-phenyl-benzimidazol-2-yl)butanoate (2.21 g, 4.17 mmol) in 1,4-dioxane (30 mL) containing water (10 mL) was added LiOH—H₂O (0.699 g, 16.7 mmol). The reaction was stirred at 50° C. for 40 minutes and at room temperature diluted with ethyl acetate and washed by 5% citric acid. The organic phase was dried over MgSO₄, filtered and the solvent was evaporated to give the title compound (1.83 g, 0.00415 mol, quantitative yield). HPLC purity: 80%, MS (ESI+) m/z=441 [M+H]⁺. The material was used as such in next step.

### Ethyl (2S)-2-[[(2S)-2-(tert-butoxycarbonylamino)-4-(5-nitro-1-phenyl-benzimidazol-2-yl) butanoyl]amino]-4-methyl-pentanoate (compound 13-9)

HATU (1.74 g, 0.00457 mol) was added to a mixture of (2S)-2-(tert-butoxycarbonylamino)-4-(5-nitro-1-phenyl-benzimidazol-2-yl)butanoic acid (1.83 g, 0.00415 mol), ethyl (2S)-2-amino-4-methyl-pentanoate; hydrochloride (0.894 g, 0.00457 mol) and triethylamine (2.32 mL, 0.0166 mol) in DMF (30 mL). The reaction was stirred for 1 hour. Toluene was added and the mixture was washed by 5% citric acid and brine. The organic phase was dried over MgSO₄, filtered and the solvent was evaporated to give the title compound (1.82 g, 0.00312 mol, 75% yield). HPLC purity: 95%, MS (ESI+) m/z=582 [M+H]⁺. The material was used as such in next step.

### Ethyl (2S)-2-[[(2S)-4-(5-amino-1-phenyl-benzimidazol-2-yl)-2-(tert-butoxycarbonyl amino)butanoyl]amino]-4-methyl-pentanoate (compound 13-10)

A mixture of ethyl (2S)-2-[[(2S)-2-(tert-butoxycarbonylamino)-4-(5-nitro-1-phenyl-benzimidazol-2-yl)butanoyl]amino]-4-methyl-pentanoate (1.81 g, 0.00311 mol) and Pd/C 10% (0.331 g, 0.00311 mol) in ethanol (90 mL) was stirred under H₂ atmosphere for 4 hours. Solid material was filtered of and the solvent was evaporated to give the title compound (1.71 g, 0.00311 mol, quantitative yield). The material was used as such in next step. HPLC purity: 93%, MS (ESI+) m/z=552 [M+H]⁺.

¹H NMR (400 MHz, DMSO-d6) δ 8.26 (d, J=7.6 Hz, 1H), 7.60 (t, J=7.5 Hz, 2H), 7.55-7.49 (m, 1H), 7.43 (d, J=7.0 Hz, 2H), 6.92 (d, J=8.2 Hz, 1H), 6.80-6.75 (m, 2H), 6.52 (dd, J=8.5, 2.1 Hz, 1H), 4.81 (s, 2H), 4.24 (ddd, J=9.9, 7.6, 5.0 Hz, 1H), 4.09-3.96 (m, 3H), 2.77-2.64 (m, 2H), 2.20-2.09 (m, 1H), 1.99-1.87 (m, 1H), 1.70-1.44 (m, 3H), 1.33 (s, 9H), 1.14 (t, J=7.1 Hz, 3H), 0.89 (d, J=6.6 Hz, 3H), 0.82 (d, J=6.5 Hz, 3H).

### Ethyl (2S)-2-[[(2S)-4-[5-[bis(2-chloroethyl)amino]-1-phenyl-benzimidazol-2-yl]-2-(tert-butoxycarbonylamino)butanoyl]amino]-4-methyl-pentanoate (compound 13-11)

NaBH₃CN (0.137 g, 0.00218 mol) was added to a stirred mixture of ethyl (2S)-2-[[(2S)-4-(5-amino-1-phenyl-benzimidazol-2-yl)-2-(tert-butoxycarbonylamino)butanoyl]amino]-4-methyl-pentanoate (0.300 g, 0.000544 mol), 2-chloroacetaldehyde (50.0% in water, 0.344 mL, 0.00272 mol) and TFA (0.242 mL, 3.26 mmol) in EtOH (6 mL) at room temperature. After 20 minutes additional 2-chloroacetaldehyde (50% in water, 0.344 mL, 2.72 mmol) and NaBH₃CN (0.137 g, 2.18 mmol) was added. Finally, after 10 minutes additional 2-chloroacetaldehyde (50% in water, 0.344 mL, 2.72 mmol), NaBH₃CN (0.137 g, 2.18 mmol) and TFA (0.242 mL, 3.26 mmol) was added. Ethyl acetate was added to the mixture and the organic phase was washed by saturated sodium bicarbonate and brine. The compound was flash chromatographed twice on silica first eluting with 2% EtOH in DCM and with 20 to 30% ethyl acetate in petroleum ether to give the title compound (0.158 g, 0.00234 mol, 43% yield). HPLC purity: 97%, MS (ESI+) m/z=676 [M+H]⁺.

### Ethyl (2S)-2-[[(2S)-2-amino-4-[5-[bis(2-chloroethyl)amino]-1-phenyl-benzimidazol-2-yl]butanoyl]amino]-4-methyl-pentanoate; hydrochloride (Example compound 13)

Ethyl (2S)-2-[[(2S)-4-[5-[bis(2-chloroethyl)amino]-1-phenyl-benzimidazol-2-yl]-2-(tert-butoxycarbonylamino)butanoyl]amino]-4-methyl-pentanoate (190 mg, 0.281 mmol) was treated with TFA (2 mL) in DCM (2 mL). The solvent was evaporated, and the crude residue was purified by preparative HPLC, with several injections, performed on a Gilson system equipped with a UV detector using an ACE 5 C8 Prep, 100*21.2 mm column (0.1% TFA in water) gradient 25-50% acetonitrile. Pure fractions were pooled giving 750 mL solution. To the ice-cold solution was added 12 M HCl (3 mL) to give a 0.05 M HCl solution. The solvent was removed by lyophilization to give the title compound (Example compound 13) (92 mg, 0.159 mmol, 53% yield). MS (ESI+) m/z 576 [M+H]⁺.

¹H NMR (400 MHz, DMSO-d6) δ 9.04 (d, J=7.1 Hz, 1H), 8.54-8.42 (m, 3H), 7.73-7.62 (m, 4H), 7.16-7.01 (m, 3H), 4.28-4.20 (m, 2H), 4.06-3.97 (m, 3H), 3.88-3.73 (m, 8H), 3.14 (t, J=7.2 Hz, 2H), 2.41-2.24 (m, 2H), 1.76-1.65 (m, 1H), 1.61-1.46 (m, 2H), 1.13 (t, J=7.1 Hz, 3H), 0.89 (dd, J=18.0, 6.5 Hz, 6H).

### Example 14: Methyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 14)

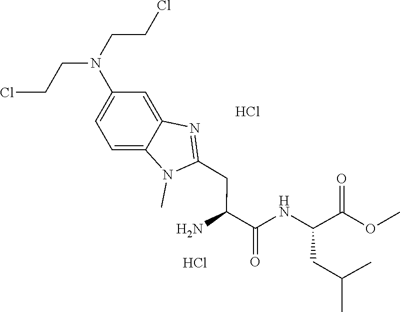 [SMILES: COC(=O)[C@H](CC(C)C)NC(=O)[C@@H](N)Cc1nc2cc(N(CCCl)CCCl)ccc2n1C ; Cl ; Cl]

Ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4) (11 mg, 0.0182 mmol) was dissolved in MeOH (2 mL). One drop of HCl was added and the reaction mixture was stirred at 70° C. overnight. The sample was purified by preparative chromatography (acidic buffer; flow 25 mL/min, gradient over 15 min from 10% B to 40% B. Fractions were collected based on the UV-signal at 238 nm). HCl in dioxane (4M, 0.01 mL) was added to the combined fractions and the sample was immediately lyophilized to afford methyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 14) (3.5 mg, 0.00619 mmol, 34% yield) as a yellow solid. HPLC purity: 99%; MS (ESI+) m/z=486 [M+H]⁺.

¹H NMR (600 MHz, DMSO-d6) δ 14.8 (s, 1H), 9.09 (d, J=7.6 Hz, 1H), 8.59 (s, 3H), 7.62 (s, 1H), 6.89 (d, J=2.4 Hz, 1H), 6.54 (s, 1H), 4.37 (dt, J=31.5, 7.6 Hz, 2H), 3.93-3.70 (m, 11H), 3.54 (s, 3H), 2.51 (t, J=5.6 Hz, 1H), 1.63 (dq, J=12.5, 6.3 Hz, 1H), 1.56-1.45 (m, 2H), 1.31-1.17 (m, 1H), 0.85 (dd, J=13.8, 6.5 Hz, 6H).

### Example 15: Isopropyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 15)

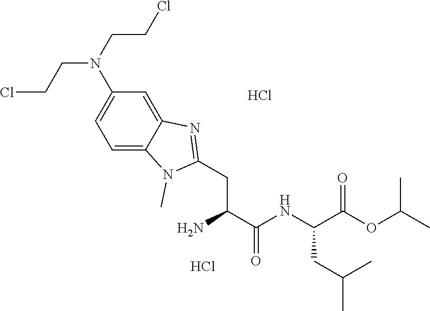 [SMILES: CC(C)C[C@H](NC(=O)[C@@H](N)Cc1nc2cc(N(CCCl)CCCl)ccc2n1C)C(=O)OC(C)C ; Cl ; Cl]

Ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4) (10 mg, 0.0166 mmol) was dissolved in isopropyl alcohol (2 mL). One drop of HCl was added and the reaction mixture was stirred at 70° C. overnight. The sample was purified by preparative chromatography (acidic buffer; flow 25 mL/min, gradient over 15 min from 11% B to 42% B. Fractions were collected based on the UV-signal at 239 nm). HCl in dioxane (4M, 0.01 mL) was added to the combined fractions and the sample was immediately lyophilized to afford isopropyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 15) (4.8 mg, 0.00793 mmol, 48% yield) as a yellow solid. HPLC purity: 97%; MS (ESI+) m/z=514 [M+H]⁺.

¹H NMR (600 MHz, DMSO-d6) δ 14.60 (s, 1H), 9.14 (d, J=7.6 Hz, 1H), 8.68 (s, 3H), 7.71 (s, 1H), 7.11 (s, 1H), 6.89 (d, J=2.4 Hz, 1H), 4.83 (h, J=6.3 Hz, 1H), 4.44 (t, J=6.9 Hz, 1H), 4.28 (ddd, J=9.8, 7.6, 5.2 Hz, 1H), 3.91 (s, 3H), 3.81 (dt, J=12.4, 6.9 Hz, 4H), 3.75 (dd, J=9.9, 4.8 Hz, 4H), 3.58 (s, 2H), 1.68 (ddt, J=15.3, 12.9, 6.6 Hz, 1H), 1.50 (qdd, J=13.8, 9.3, 5.3 Hz, 2H), 1.14 (dd, J=36.1, 6.3 Hz, 6H), 0.86 (dd, J=17.3, 6.6 Hz, 6H).

### Example 16: Ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate; dihydrochloride (Example compound 16)

Example 16 was synthesized according to Scheme 5 below:

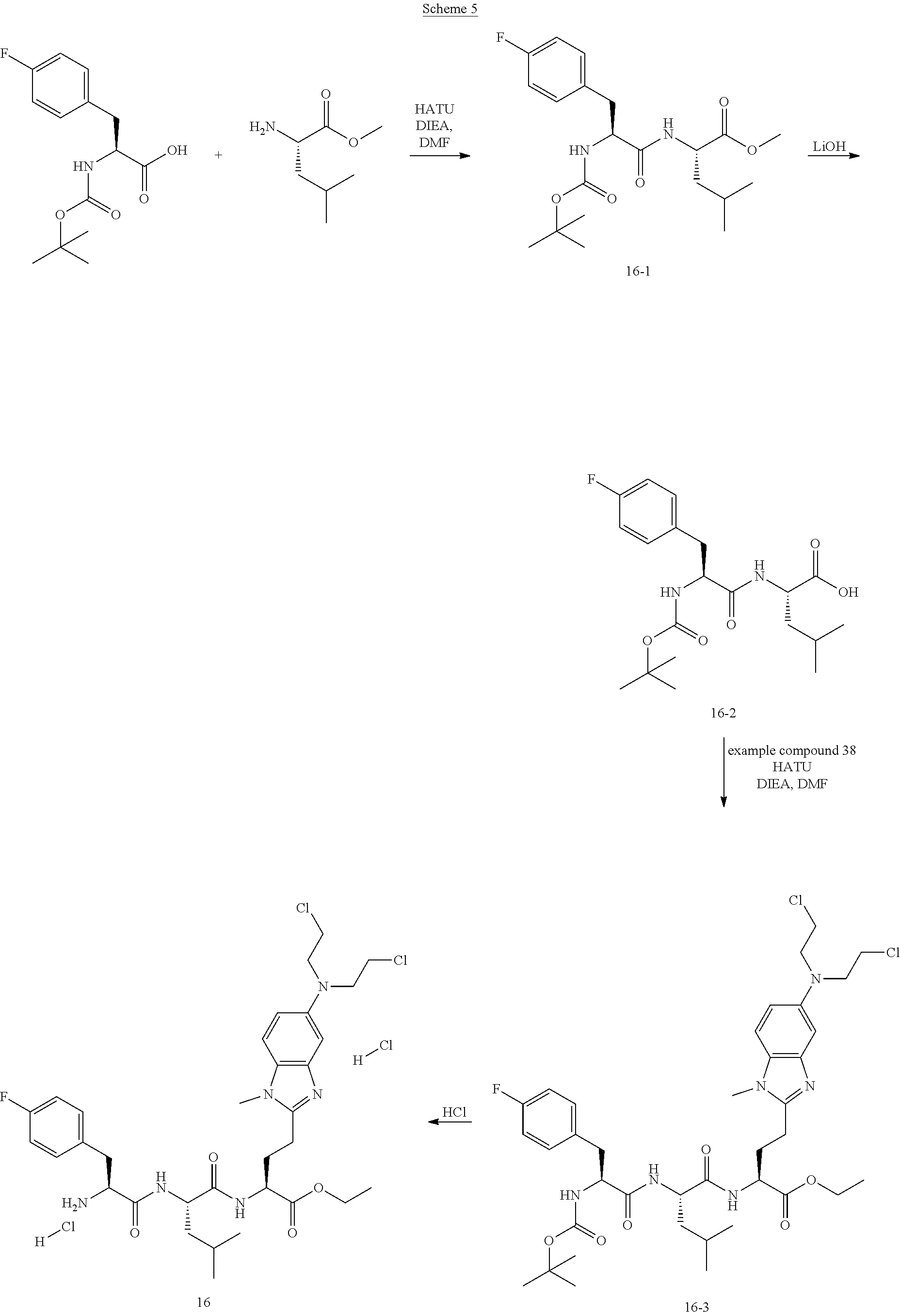

### (2S)-2-[[(2S)-2-(tert-Butoxycarbonylamino)-3-(4-fluorophenyl)propanoyl]amino]-4-methyl-pentanoate (compound 16-1)

To a solution of (2S)-2-(tert-butoxycarbonylamino)-3-(4-fluorophenyl)propanoic acid (0.700 g, 0.00247 mol) in DMF (5.0 mL) at rt was added HATU (0.940 g, 0.00247 mol) and DIEA (0.423 mL, 0.00247 mol), followed by methyl (2S)-2-amino-4-methyl-pentanoate (0.395 g, 0.00272 mol). The resulting mixture was stirred at room temperature overnight. The reaction mixture was diluted with H₂O and extracted with DCM. The organic layer was dried (Na₂SO₄) and concentrated in vacuo below 20° C. The crude was purified with a BUCHI automatic flash system (10 g column, EtOAc/petroleum ether) to afford methyl (2S)-2-[[(2S)-2-(tert-butoxycarbonylamino)-3-(4-fluorophenyl)propanoyl]amino]-4-methyl-pentanoate (0.420 g, 1.02 mmol, 41% yield) as a white solid. HPLC purity: 75%; MS (ESI+) m/z=411 [M+H]⁺.

### (2S)-2-[[(2S)-2-(tert-Butoxycarbonylamino)-3-(4-fluorophenyl)propanoyl]amino]-4-methyl-pentanoic acid (compound 16-2)

To a solution of methyl (2S)-2-[[(2S)-2-(tert-butoxycarbonylamino)-3-(4-fluorophenyl)propanoyl]amino]-4-methyl-pentanoate (420 mg, 1.02 mmol) in THF (1.5 mL) was added a solution of LiOH—H₂O (47.2 mg, 1.13 mmol) in water (0.5 mL). The mixture was stirred at room temperature for 1h. The precipitated solid was filtered and dried to afford (2S)-2-[[(2S)-2-(tert-butoxycarbonylamino)-3-(4-fluorophenyl)propanoyl]amino]-4-methyl-pentanoic acid (350 mg, 0.883 mmol, 86% yield) as a white solid. HPLC purity: 85%; MS (ESI+) m/z=341 [M+H-tBu]⁺.

### Ethyl (2S)-4-[5-[bis(2-chloroethyl)amino]-1-methyl-benzimidazol-2-yl]-2-[[(2S)-2-[[(2S)-2-(tert-butoxycarbonylamino)-3-(4-fluorophenyl)propanoyl]amino]-4-methyl-pentanoyl]amino]butanoate (compound 16-3)

To a solution of (2S)-2-[[(2S)-2-(tert-butoxycarbonylamino)-3-(4-fluorophenyl)propanoyl]amino]-4-methyl-pentanoic acid (95.0 mg, 0.240 mmol) in DMF (3.0 mL) at room temperature was added HATU (91.1 mg, 0.240 mmol), DIEA (0.0820 mL, 0.479 mmol and ethyl (2S)-2-amino-4-[5-[bis(2-chloroethyl)amino]-1-methyl-benzimidazol-2-yl]butanoate; dihydrochloride (Example compound 38) (125 mg, 0.264 mmol). The resulting mixture was stirred at room temperature overnight. The reaction mixture was diluted with H₂O and extracted with DCM using phase separator. The organic layer was dried (Na₂SO₄) and concentrated in vacuo below 20° C. The crude was purified by preparative HPLC (acidic buffer; flow 25 mL/min, gradient over 15 min from 31% B to 57% B. Fractions were collected based on the UV-signal at 235 nm) to afford ethyl (2S)-4-[5-[bis(2-chloroethyl)amino]-1-methyl-benzimidazol-2-yl]-2-[[(2S)-2-[[(2S)-2-(tert-butoxycarbonylamino)-3-(4-fluorophenyl)propanoyl]amino]-4-methyl-pentanoyl]amino]butanoate (80.0 mg, 0.103 mmol, yield: 43%) as an off-white solid. HPLC purity: 100%; MS (ESI+) m/z=779 [M+H]⁺.

### Ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate; dihydrochloride (Example compound 16)

To ethyl (2S)-4-[5-[bis(2-chloroethyl)amino]-1-methyl-benzimidazol-2-yl]-2-[[(2S)-2-[[(2S)-2-(tert-butoxycarbonylamino)-3-(4-fluorophenyl)propanoyl]amino]-4-methyl-pentanoyl]amino]butanoate (80.0 mg, 0.103 mmol) in EtOH (1 mL), HCl in dioxane (4.00 mol/L, 0.128 mL, 0.513 mmol) was added and the reaction mixture was stirred overnight. After completion of the reaction, solvent was evaporated under ca 15° C. and it was immediately submitted for preparative HPLC (acidic buffer; flow 25 mL/min, gradient over 15 min from 12% B to 44% B. Fractions were collected based on the UV-signal at 235 nm). All collected pure HPLC fractions kept on Mivacuum to evaporate acetonitrile, and HCl in dioxane (4M, 0.01 mL) was added to the resulted fractions pooled. The mixture was frozen immediately in a dry ice cool bath and left on freeze drier to afford ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate; dihydrochloride (Example compound 16) (6.00 mg, 0.00797 mmol, 8% yield) as a white powder. HPLC purity: 95%; MS (ESI+) m/z=679 [M+H]⁺.

¹H NMR (600 MHz, DMSO-d6) δ 14.4 (br s, 1H), 8.80-8.71 (m, 2H), 8.24-8.01 (m, 3H), 7.36-7.25 (m, 2H), 7.18-7.08 (m, 2H), 6.97-6.82 (m, 1H), 4.53-4.32 (m, 2H), 4.19-3.99 (m, 3H), 3.90-3.67 (m, 12H), 3.09 (m, 4H), 2.88 (dd, J=14.2, 8.3 Hz, 1H), 2.38-2.26 (m, 1H), 2.21-2.09 (m, 1H), 1.67 (dq, J=13.5, 6.6 Hz, 1H), 1.55-1.43 (m, 2H), 1.17 (t, J=7.1 Hz, 3H), 0.93 (d, J=6.6 Hz, 3H), 0.89 (d, J=6.6 Hz, 3H).

### Example 17: (2S)-2-amino-3-[5-[bis(2-chloroethyl)amino]-1-methyl-benzimidazol-2-yl]propanoic acid; dihydrochloride (Example compound 17)

Example 17 was synthesized according to Scheme 6 below:

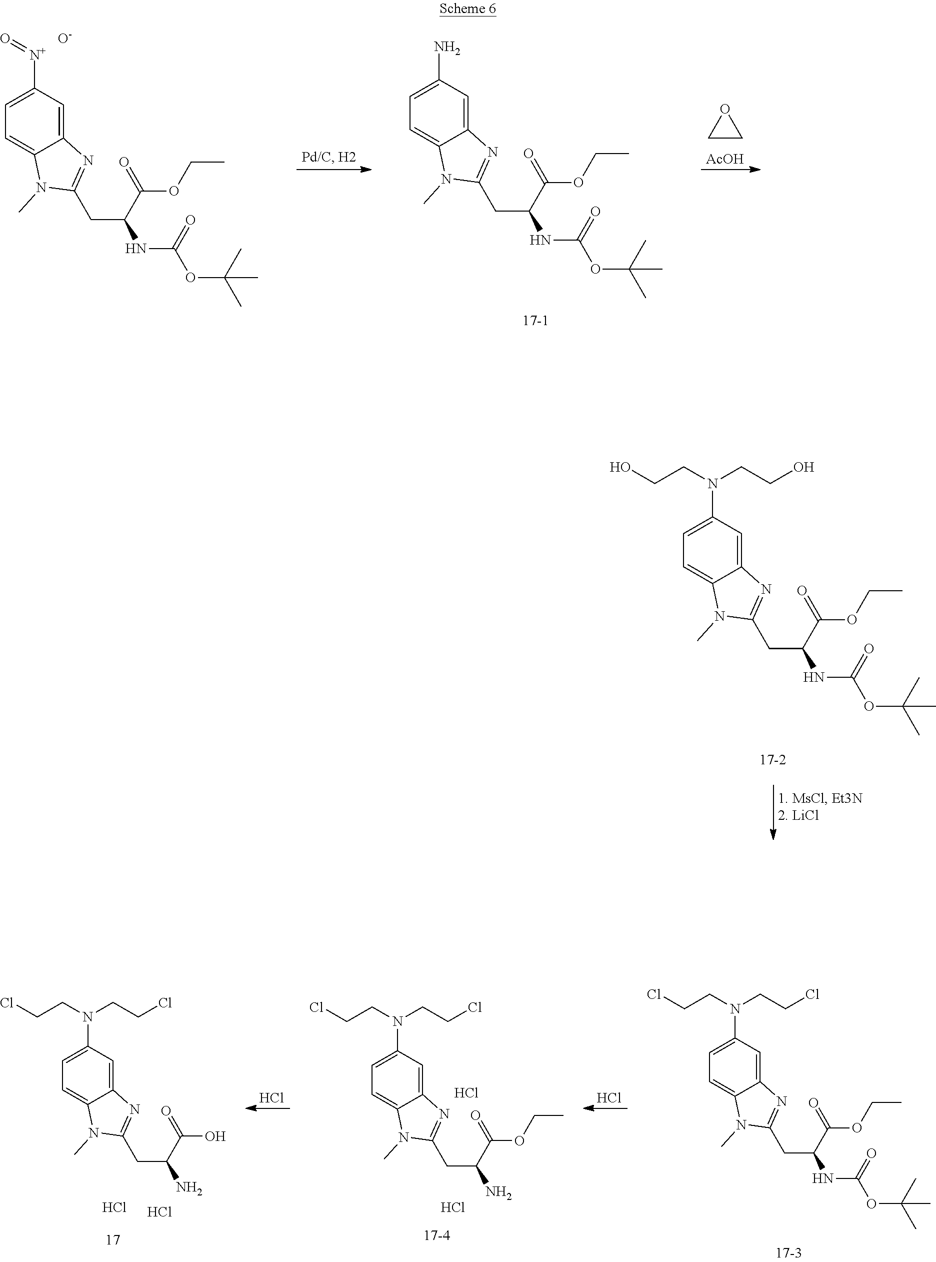

### Ethyl (2S)-3-(5-amino-1-methyl-benzimidazol-2-yl)-2-(tert-butoxycarbonylamino)propanoate (compound 17-1)

Ethyl (2S)-2-(tert-butoxycarbonylamino)-3-(1-methyl-5-nitro-benzimidazol-2-yl)propanoate (Scheme 1) (573 mg, 1.42 mmol) was dissolved in EtOH (10 mL) and placed in a H-reactor. 10% Pd/C (75.4 mg) was added, and the reactor was sealed. A nitrogen purge was conducted. H₂ was charged to the reactor at 3 bar and the reaction was stirred at 35° C. for 3h. Hydrogen was vented with a nitrogen purge. Catalyst was removed by filtration through Celite. The solvent was evaporated and the crude ethyl (2S)-3-(5-amino-1-methyl-benzimidazol-2-yl)-2-(tert-butoxycarbonylamino)propanoate (421 mg, 1.13 mmol, 74% yield) was obtained as a yellow solid, which was used in the next step without further purification. HPLC purity: 90%; MS (ESI+) m/z=363 [M+H]⁺.

### Ethyl (2S)-3-[5-[bis(2-hydroxyethyl)amino]-1-methyl-benzimidazol-2-yl]-2-(tert-butoxycarbonylamino)propanoate (compound 17-2)

Ethyl (2S)-3-(5-amino-1-methyl-benzimidazol-2-yl)-2-(tert-butoxycarbonylamino)propanoate (421 mg, 1.13 mmol) was dissolved in acetic acid (5 mL). Oxirane (2.3M, 4.55 mL) was added and the reaction was stirred at room temperature for 24 hours. The solvent was evaporated and the crude material was dissolved in DCM, and washed with saturated aqueous NaHCO₃. The organic layer was washed with water and brine and passed through a phase separator. The material was concentrated to afford ethyl (2S)-3-[5-[bis(2-hydroxyethyl)amino]-1-methyl-benzimidazol-2-yl]-2-(tert-butoxycarbonylamino)propanoate (0.421 g, 0.888 mmol, 85% yield) as a brown oil, which was sufficiently pure to be used in the next step. HPLC purity: 95%; MS (ESI+) m/z=451 [M+H]⁺.

### Ethyl (2S)-3-[5-[bis(2-chloroethyl)amino]-1-methyl-benzimidazol-2-yl]-2-(tert-butoxycarbonylamino)propanoate (compound 17-3)

To a solution of ethyl (2S)-3-[5-[bis(2-hydroxyethyl)amino]-1-methyl-benzimidazol-2-yl]-2-(tert-butoxycarbonylamino)propanoate (421 g, 0.888 mmol, 95% purity) and triethylamine (0.495 mL, 3.55 mmol) in DCM (10 mL) was added methane sulfonyl chloride (0.213 mL, 2.75 mmol) and the reaction mixture was stirred at room temperature for 20 min. The reaction mixture was washed with a mixture of NaHCO₃(sat):water (2:5) (50 mL), 0.3 M HCl (50 mL) and brine (50 mL). The organic phase was passed through a phase separator and evaporated under reduced pressure. The crude ethyl (2S)-3-[5-[bis(2-methylsulfonyloxyethyl)amino]-1-methyl-benzimidazol-2-yl]-2-(tert-butoxycarbonylamino)propanoate was immediately used in the next step without further purification.

Ethyl (2S)-3-[5-[bis(2-methylsulfonyloxyethyl)amino]-1-methyl-benzimidazol-2-yl]-2-(tert-butoxycarbonylamino)propanoate (521 mg, 0.79 mmol, 92% purity) was dissolved in DMF (5 mL). LiCl (0.502 g, 11.9 mmol) was added, and the reaction was heated at 60° C. for 2h. The reaction mixture was diluted with a mixture of toluene (10 mL) and EtOAc (10 mL), then washed with brine (3×20 mL), dried over MgSO₄, filtered and evaporated. The crude obtained was purified by flash chromatography using a gradient of ethyl acetate in DCM from 0-20%. The fractions containing the desired compound were combined and concentrated to afford of ethyl (2S)-3-[5-[bis(2-chloroethyl)amino]-1-methyl-benzimidazol-2-yl]-2-(tert-butoxycarbonylamino)propanoate (375 mg, 0.692 mmol, 88% yield) as a brown oil. HPLC purity: 90%; MS (ESI+) m/z=487 [M+H]⁺ HPLC purity (mesylated intermediate): 95%; MS (ESI+) m/z=607 [M+H]⁺.

### Ethyl (2S)-2-amino-3-[5-[bis(2-chloroethyl)amino]-1-methyl-benzimidazol-2-yl]propanoate; dihydrochloride (compound 17-4)

To a solution of ethyl (2S)-3-[5-[bis(2-chloroethyl)amino]-1-methyl-benzimidazol-2-yl]-2-(tert-butoxycarbonylamino)propanoate (375 mg, 0.692 mmol, 90% purity) in EtOH (5 mL) was added hydrogen chloride in dioxane (4.00 mol/L, 1.73 mL, 6.92 mmol) and stirred at 25° C. for 3h. The solvent was evaporated under reduced pressure to afford ethyl (2S)-2-amino-3-[5-[bis(2-chloroethyl)amino]-1-methyl-benzimidazol-2-yl]propanoate; dihydrochloride (0.307 g, 0.660 mmol, 95% yield) as an off-white solid. HPLC purity: 99%; MS (ESI+) m/z=387 [M+H]⁺.

### (2S)-2-Amino-3-[5-[bis(2-chloroethyl)amino]-1-methyl-benzimidazol-2-yl]propanoic acid; dihydrochloride (Example compound 17)

Ethyl (2S)-2-amino-3-[5-[bis(2-chloroethyl)amino]-1-methyl-benzimidazol-2-yl]propanoate (30 mg, 0.064 mmol) and 5N HCl (0.025 mL) were stirred at room temperature for 4 hours. The temperature was slowly increased to 60° C. The temperature was raised to 100° C. and stirred for 30 minutes. The reaction was allowed to cool to rt and the volatiles were removed under vacuum. The crude was purified by preparative HPLC (acidic buffer; flow 25 mL/min, gradient over 15 min from 1% B to 28% B. Fractions were collected based on the UV-signal at 236 nm) to afford (2S)-2-amino-3-[5-[bis(2-chloroethyl)amino]-1-methyl-benzimidazol-2-yl]propanoic acid; dihydrochloride (Example compound 17) (8 mg, 0.022 mmol, 26% yield) as a pale light pink solid. HPLC purity: 95%; MS (ESI+) m/z=359 [M+H]⁺.

¹H NMR (600 MHz, DMSO-d6) δ 14.90 (s, 1H), 8.93 (s, 3H), 7.75 (d, J=9.2 Hz, 1H), 7.15 (dd, J=9.3, 2.4 Hz, 1H), 6.91 (d, J=2.3 Hz, 1H), 4.65 (t, J=7.6 Hz, 1H), 3.84-3.71 (m, 13H).

### Example 18: (S)-2-amino-4-(5-(bis(2-chloroethyl)amino)-1-methyl-1H-benzo[d]imidazol-2-yl)butanoic acid dihydrochloride (Example compound 18)

Example 18 was synthesized according to Scheme 7 below:

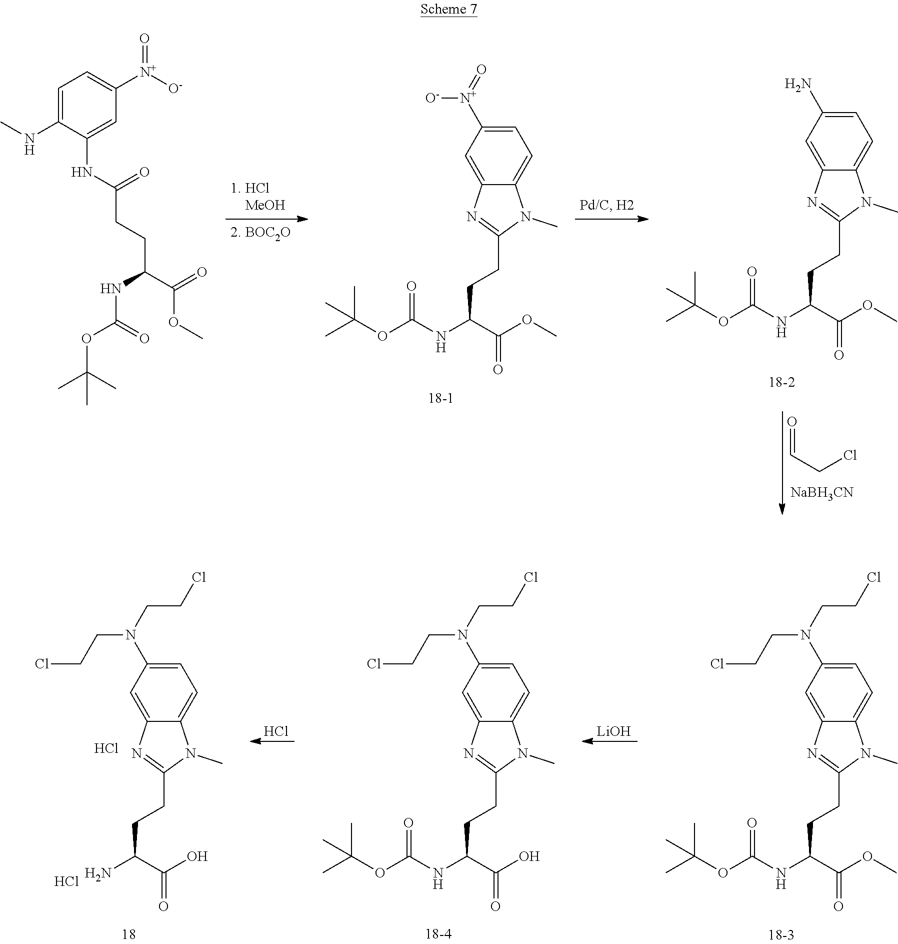

### Ethyl (2S)-2-(tert-butoxycarbonylamino)-4-(1-methyl-5-nitro-benzimidazol-2-yl)butanoate (compound 18-1)

To a solution of methyl (2S)-2-(tert-butoxycarbonylamino)-5-[2-(methylamino)-5-nitro-anilino]-5-oxo-pentanoate (Scheme 1) (50 g, 98.2 mmol) in MeOH (150 mL) was added aqueous hydrogen chloride (12.0 mol/L, 73.6 mL, 884 mmol) and the mixture was stirred at 70° C. for 30 minutes. The reaction mixture was dissolved with water (500 mL) and washed with EtOAc (2×150 mL). The aqueous phase was mixed with EtOAc (300 mL) and neutralized with solid K₂CO₃ until pH 9-10. The aqueous phase was extracted with EtOAc (2×100 mL). The organics were dried over MgSO₄, filtered and evaporated under reduced pressure to afford methyl (2S)-2-amino-4-(1-methyl-5-nitro-benzimidazol-2-yl)butanoate (26.0 g, 82.7 mmol, 84% yield) as a reddish solid. The crude was used in the next step without further purification. HPLC purity: 93%; MS (ESI+) m/z=293 [M+H]⁺.

To a solution of methyl (2S)-2-amino-4-(1-methyl-5-nitro-benzimidazol-2-yl)butanoate (26 g, 82.7 mmol) in THF (100 mL) was added triethylamine (13.8 mL, 99.3 mmol) and tert-butoxycarbonyl tert-butyl carbonate (20.9 mL, 91 mmol) and the mixture was stirred at room temperature overnight. The crude was concentrated under reduced pressure and dissolved with EtOAc (400 mL) and washed with 0.5N HCl (400 mL). The phases were separated and the aqueous phase was extracted with EtOAc (2×200 mL). The organics were dried over MgSO4, filtered and evaporated under reduced pressure. The crude was dissolved in a small amount of THF and Et₂O was added. The mixture was cooled to 0° C. and let it stand for 30 min. The solid was filtered and washed with cold Et₂O. The solid was dried under reduced pressure to afford ethyl (2S)-2-(tert-butoxycarbonylamino)-4-(1-methyl-5-nitro-benzimidazol-2-yl)butanoate (27 g, 68.8 mmol, 83% yield) as an off-white solid. HPLC purity: 100%; MS (ESI+) m/z=393 [M+H]⁺.

### Methyl (2S)-4-(5-amino-1-methyl-benzimidazol-2-yl)-2-(tert-butoxycarbonylamino)butanoate (compound 18-2)

Methyl (2S)-2-(tert-butoxycarbonylamino)-4-(1-methyl-5-nitro-benzimidazol-2-yl)butanoate (1.95 g, 4.97 mmol) was dissolved in MeOH (15 mL) and placed in a H-reactor. 10% Pd/C (264 mg) was added, and the reactor was sealed. A nitrogen purge was conducted. H₂ was charged to the reactor at 3 bar and the reaction was stirred at 35° C. for 2h. Hydrogen was vented with a nitrogen purge. Catalyst was removed by filtration through Celite. The solvent was evaporated and the crude methyl (2S)-4-(5-amino-1-methyl-benzimidazol-2-yl)-2-(tert-butoxycarbonylamino)butanoate (1.8 g, quantitative yield) was obtained as an off-white (slightly pink) solid, which was used in the next step without further purification. HPLC purity: 95%; MS (ESI+) m/z 363 [M+H]⁺.

### Methyl (2S)-4-[5-[bis(2-chloroethyl)amino]-1-methyl-benzimidazol-2-yl]-2-(tert-butoxycarbonylamino)butanoate (compound 18-3)

2-Chloroacetaldehyde (1.30 g, 1.05 mL, 8.28 mmol, 50%, 10 eq.) was added to a solution of methyl (2S)-4-(5-amino-1-methyl-benzimidazol-2-yl)-2-(tert-butoxycarbonylamino) butanoate (300 mg, 0.83 mmol), TFA (0.369 mL, 4.97 mmol, 6 eq.) and MeOH (10 mL). The reaction mixture was stirred at 0° C. for 15 min, then NaBH₃CN (104 mg, 1.66 mmol, 2 eq.) was added over a period of 5 min and the mixture was stirred at 0° C. for 20 min. Water (10 mL), NaHCO₃ (10 mL) and EtOAc (10 mL) were added and the layers were partitioned. The aqueous layer was extracted with EtOAc (3×10 mL). Combined organic layers were washed by brine (2×20 mL), dried over anhydrous MgSO₄, filtered, and concentrated under reduced pressure. The crude residue was purified on a silica column eluting the product with 80% EtOAc in p-ether. The fractions were pooled and concentrated to give methyl (2S)-4-[5-[bis(2-chloroethyl)amino]-1-methyl-benzimidazol-2-yl]-2-(tert-butoxycarbonylamino)butanoate (207 mg, 0.425 mmol, 51% yield). HPLC purity: 93%; MS (ESI+) m/z 487 [M+H]⁺.

### (2S)-4-[5-[Bis(2-chloroethyl)amino]-1-methyl-benzimidazol-2-yl]-2-(tert-butoxycarbonyl-amino)butanoic acid (compound 18-4)

A mixture of methyl (2S)-4-[5-[bis(2-chloroethyl)amino]-1-methyl-benzimidazol-2-yl]-2-(tert-butoxycarbonylamino)butanoate (207 mg, 0.425 mmol) and LiOH—H₂O (54 mg, 1.27 mmol) were suspended in 1,4-dioxane (12 mL) and water (4 mL). The reaction mixture was heated at 50° C. for 20 min. The solution was allowed to attain rt and water (20 mL) was added. Then, the mixture was acidified with 1 M HCl to pH 5 and the product was extracted with EtOAc (3×70 mL). The combined organic layers were washed with brine (100 mL), dried over anhydrous MgSO₄, filtered and concentrated on a rotary evaporator to give (2S)-4-[5-[bis(2-chloroethyl)amino]-1-methyl-benzimidazol-2-yl]-2-(tert-butoxycarbonyl-amino)butanoic acid (188 mg, 0.397 mmol, 94% yield). HPLC purity: 95%; MS (ESI+) m/z 473 [M+H]⁺.

### (S)-2-amino-4-(5-(bis(2-chloroethyl)amino)-1-methyl-1H-benzo[d]imidazol-2-yl)butanoic acid dihydrochloride (Example compound 18)

(2S)-4-[5-[Bis(2-chloroethyl)amino]-1-methyl-benzimidazol-2-yl]-2-(tert-butoxycarbonyl-amino)butanoic acid (188 mg, 0.397 mmol) was dissolved in dichloromethane (3 mL) and 4 M HCl in dioxane (1.0 mL) was added dropwise. The reaction mixture was stirred at rt for 3 h, then diethyl ether (15 mL) was added. Upon the addition of diethyl ether, a precipitate was formed which was isolated by filtration and dried in vacuo to afford (S)-2-amino-4-(5-(bis(2-chloroethyl)amino)-1-methyl-1H-benzo[d]imidazol-2-yl)butanoic acid dihydrochloride (Example compound 18) (118 mg, 0.288 mmol, 73% yield). HPLC purity: 95%; MS (ESI+) m/z 373 [M+H]⁺.

¹H NMR (600 MHz, CD₃OD) δ 7.71 (d, J=9.3 Hz, 1H), 7.18 (dd, J=9.3, 2.4 Hz, 1H), 6.97 (d, J=2.4 Hz, 1H), 4.23 (dd, J=7.3, 5.5 Hz, 1H), 3.99 (s, 3H), 3.89 (t, J=6.8 Hz, 4H), 3.76 (t, J=6.7 Hz, 4H), 3.54-3.44 (m, 1H), 3.44-3.35 (m, 1H), 2.61-2.49 (m, 1H), 2.49-2.37 (m, 1H).

### Example 19: (2S)-2-[[(2S)-2-amino-4-[5-[bis(2-chloroethyl)amino]-1-methyl-benzimidazol-2-yl]butanoyl]amino]-3-methyl-butanoic acid; dihydrochloride (Example compound 19)

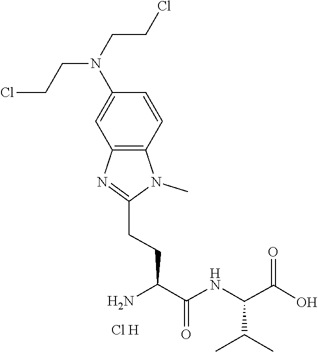

To a solution of ethyl (2S)-2-[[(2S)-2-amino-4-[5-[bis(2-chloroethyl)amino]-1-methyl-benzimidazol-2-yl]butanoyl]amino]-3-methyl-butanoate; dihydrochloride (Example compound 3) (15.3 mg, 0.0264 mmol) in THF:MeOH (2:1, 1 mL) was added a solution of lithium hydroxide monohydrate in water (0.5 M, 0.0555 mL). The mixture was stirred overnight at room temperature. The sample was filtered and purified by preparative chromatography (acidic buffer; flow 25 mL/min, gradient over 15 min from 2% B to 31% B. Fractions were collected based on the UV-signal at 235 nm). HCl in ethanol (2.5M, 1 mL) was added to the combined fractions and the sample was immediately lyophilized to afford (2S)-2-[[(2S)-2-amino-4-[5-[bis(2-chloroethyl)amino]-1-methyl-benzimidazol-2-yl]butanoyl]amino]-3-methyl-butanoic acid; dihydrochloride (Example compound 19) (5.8 mg, 0.0106 mmol, 40% yield) as a white solid. HPLC purity: 99%; MS (ESI+) m/z=472 [M+H]⁺.

¹H NMR (600 MHz, DMSO-d6) δ 14.72 (s, 1H), 12.91 (s, 1H), 8.86 (d, J=7.7 Hz, 1H), 8.55 (d, J=5.2 Hz, 3H), 7.73 (d, J=9.2 Hz, 1H), 7.13 (dd, J=9.4, 2.4 Hz, 1H), 6.90 (d, J=2.4 Hz, 1H), 4.19 (dd, J=7.7, 5.1 Hz, 2H), 3.86 (s, 3H), 3.81 (t, J=6.8 Hz, 4H), 3.76 (t, J=6.9 Hz, 4H), 3.31 (h, J=9.4 Hz, 2H), 2.33 (ddd, J=13.1, 9.4, 6.8 Hz, 1H), 2.23 (ddt, J=13.9, 9.5, 6.9 Hz, 1H), 2.19-2.10 (m, 1H), 0.94 (dd, J=6.9, 3.6 Hz, 6H).

### Example 20: (2S)-2-[[(2S)-2-amino-4-[5-[bis(2-chloroethyl)amino]-1-methyl-benzimidazol-2-yl]butanoyl]amino]-4-methyl-pentanoic acid; dihydrochloride (Example compound 20)

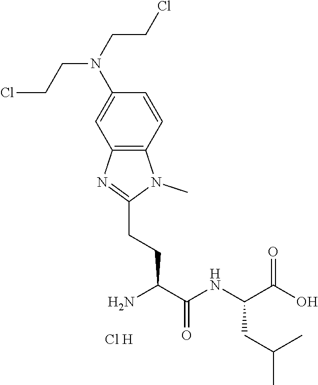

Ethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 1) (15 mg, 0.0255 mmol) was dissolved in HCl (4M in dioxane, 0.319 mL) and stirred at room temperature for 48 hours. The crude was purified by preparative chromatography (acidic buffer; flow 25 mL/min, gradient over 15 min from 9% B to 42% B. Fractions were collected based on the UV-signal at 235 nm) to afford (2S)-2-[[(2S)-2-amino-4-[5-[bis(2-chloroethyl)amino]-1-methyl-benzimidazol-2-yl]butanoyl]amino]-4-methyl-pentanoic acid; dihydrochloride (Example compound 20) (7.3 mg, 0.013 mmol, 51% yield) as a white solid. HPLC purity: 99%; MS (ESI+) m/z=486 [M+H]⁺.

¹H NMR (600 MHz, DMSO-d6) δ 14.64 (s, 1H), 12.92 (s, 1H), 9.00 (d, J=7.4 Hz, 1H), 8.49 (d, J=5.6 Hz, 3H), 7.73 (d, J=9.2 Hz, 1H), 7.13 (d, J=9.0 Hz, 1H), 6.90 (d, J=2.4 Hz, 1H), 4.26 (ddd, J=10.0, 7.3, 5.1 Hz, 1H), 4.05-3.99 (m, 1H), 3.86 (s, 3H), 3.82 (t, J=6.9 Hz, 4H), 3.76 (d, J=6.3 Hz, 4H), 3.30 (s, 2H), 2.32 (p, J=7.2 Hz, 1H), 2.26-2.19 (m, 1H), 1.76-1.69 (m, 1H), 1.57 (pt, J=9.4, 5.0 Hz, 2H), 0.89 (dd, J=24.6, 6.6 Hz, 6H).

### Example 21: Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate; dihydrochloride (Example compound 21)

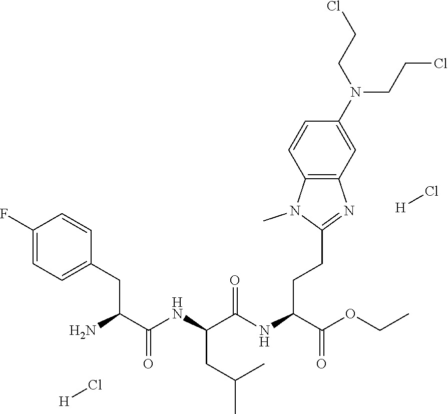 [SMILES: CCOC(=O)[C@H](CCc1nc2cc(N(CCCl)CCCl)ccc2n1C)NC(=O)[C@@H](CC(C)C)NC(=O)[C@@H](N)Cc1ccc(F)cc1 ; Cl ; Cl]

Example 21 was synthesized according to Scheme 8 below:

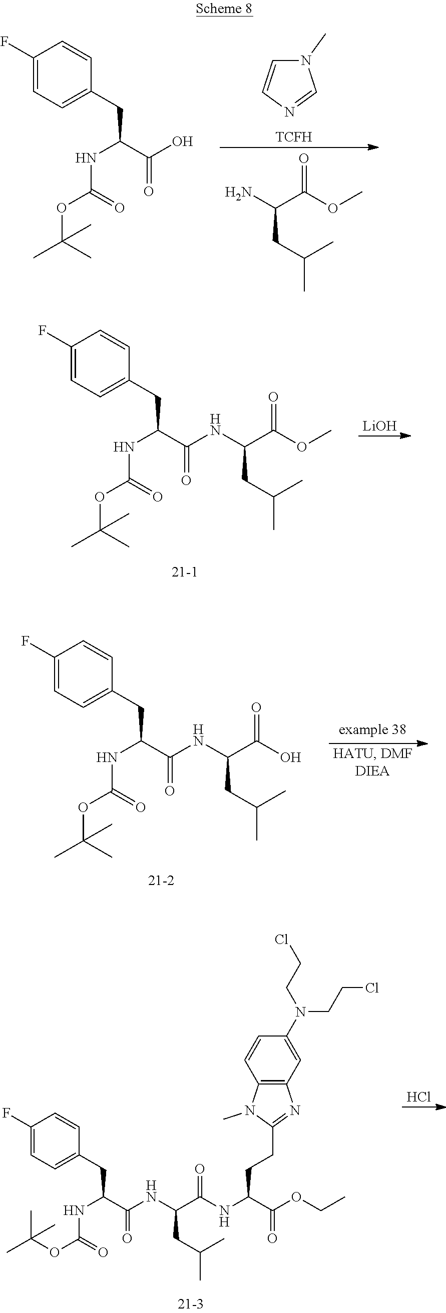

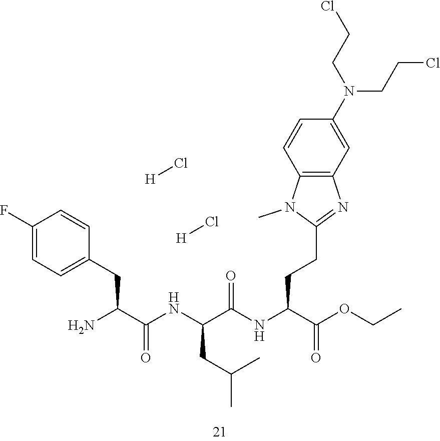 [SMILES: CCOC(=O)[C@H](CCc1nc2cc(N(CCCl)CCCl)ccc2n1C)NC(=O)[C@@H](CC(C)C)NC(=O)[C@@H](N)Cc1ccc(F)cc1 ; Cl ; Cl]

### Methyl (2R)-2-[[(2S)-2-(tert-butoxycarbonylamino)-3-(4-fluorophenyl)propanoyl]amino]-4-methyl-pentanoate (compound 21-1)

To a solution of (2S)-2-(tert-butoxycarbonylamino)-3-(4-fluorophenyl)propanoic acid (500 mg, 0.00176 mol), methyl (2R)-2-amino-4-methyl-pentanoate; hydrochloride (0.417 g, 0.00229 mol), 1-methylimidazole (0.436 mL, 0.00547 mol) in MeCN (8 mL) was added [chloro(dimethylamino)methylene]-dimethyl-ammonium; hexafluorophosphate (0.545 g, 0.00194 mol) at room temperature. The mixture was stirred at room temperature for 1h. EtOAc (10 mL) was added followed by H₂O (10 mL). The phases were separated, and the aqueous phase was washed with EtOAc (5 mL) and the combined organic phase was dried over MgSO₄, evaporated under reduced pressure. The crude product was adsorbed on Celite, and purified on Buchi Pure C-810 Flash system (20 g column, EtOAc:Petroleum Ether 0:100 to 50:50) to afford methyl (2R)-2-[[(2S)-2-(tert-butoxycarbonylamino)-3-(4-fluorophenyl)propanoyl]amino]-4-methyl-pentanoate (480 mg, 0.00117 mol, 66% yield) as a white solid. HPLC purity: 95%; MS (ESI+) m/z 411 [M+H]⁺.

### (2R)-2-[[(2S)-2-(tert-butoxycarbonylamino)-3-(4-fluorophenyl)propanoyl]amino]-4-methyl-pentanoic acid (compound 21-2)

To a solution of methyl (2R)-2-[[(2S)-2-(tert-butoxycarbonylamino)-3-(4-fluorophenyl)propanoyl]amino]-4-methyl-pentanoate (200 mg, 0.000487 mol) in THF (10 mL) was added a solution of lithium hydroxide monohydrate (0.0215 g, 0.000512 mol) in water (0.5 mL). The mixture was stirred at room temperature for 1h before addition of EtOAc (10 mL), 1N HCl (0.5 mL) and H₂O (10 mL). The phases were separated, and the aqueous phase was further extracted with EtOAc (10 mL). The combined organic phase was washed with brine (10 mL) and dried over Na₂SO₄, filtered and evaporated under reduced pressure to afford (2R)-2-[[(2S)-2-(tert-butoxycarbonylamino)-3-(4-fluorophenyl)propanoyl]amino]-4-methyl-pentanoic acid (0.185 g, 0.000467 mol, 96% yield) as a colorless sticky oil. HPLC purity: 94%; MS (ESI+) m/z 341 [M-tBu]⁺.

### Ethyl (2S)-4-[5-[bis(2-chloroethyl)amino]-1-methyl-benzimidazol-2-yl]-2-[[(2R)-2-[[(2S)-2-(tert-butoxycarbonylamino)-3-(4-fluorophenyl) propanoyl] amino]-4-methyl-pentanoyl] amino] butanoate (compound 21-3)

(2R)-2-[[(2S)-2-(tert-butoxycarbonylamino)-3-(4-fluorophenyl) propanoyl] amino]-4-methylpentanoic acid (95.0 mg, 0.240 mmol) in DMF (3.0 mL) at rt. was added HATU (91.1 mg, 0.240 mmol) and DIEA (0.0820 mL, 0.479 mmol) followed by ethyl (2S)-2-amino-4-[5-[bis(2-chloroethyl)amino]-1-methyl-benzimidazol-2-yl]butanoate; dihydrochloride (Example compound 38) (125 mg, 0.264 mmol). The resulting mixture was stirred at room temperature overnight. The reaction mixture was diluted with H₂O and extracted with DCM. The organic layer was dried (Na₂SO₄) and concentrated in vacuo and purified by preparative chromatography. The pure fractions were freeze dried to afford ethyl (2S)-4-[5-[bis(2-chloroethyl)amino]-1-methyl-benzimidazol-2-yl]-2-[[(2R)-2-[[(2S)-2-(tert-butoxycarbonylamino)-3-(4-fluorophenyl) propanoyl] amino]-4-methyl-pentanoyl] amino] butanoate (0.080 g, 0.103 mmol, 43% yield) as a white solid. HPLC purity: 97%; MS (ESI+) m/z=779 [M+H]⁺.

### Ethyl (2S)-2-[[(2R)-2-[[(2S)-2-amino-3-(4-fluorophenyl) propanoyl] amino]-4-methyl-pentanoyl] amino]-4-[5-[bis(2-chloroethyl) amino]-1-methyl-benzimidazol-2-yl] butanoate; dihydrochloride (Example compound 21)

To ethyl (2S)-4-[5-[bis(2-chloroethyl)amino]-1-methyl-benzimidazol-2-yl]-2-[[(2R)-2-[[(2S)-2-(tert-butoxycarbonylamino)-3-(4-fluorophenyl)propanoyl]amino]-4-methyl-pentanoyl]amino]butanoate (80.0 mg, 0.103 mmol) in EtOH (1 mL), HCl in dioxane (4.00 mol/L, 0.128 mL, 0.513 mmol) was added and the reaction mixture was stirred overnight. After completion of the reaction, solvent was evaporated and the crude was purified by preparative chromatography. The pure fractions were collected and freeze dried to afford ethyl (2S)-2-[[(2R)-2-[[(2S)-2-amino-3-(4-fluorophenyl) propanoyl] amino]-4-methyl-pentanoyl] amino]-4-[5-[bis(2-chloroethyl) amino]-1-methyl-benzimidazol-2-yl] butanoate; dihydrochloride (Example compound 21) (6 mg, 0.0079 mmol, 8% yield) as a white solid. HPLC purity: 99%; MS (ESI+) m/z=679 [M+H]⁺.

¹H NMR (600 MHz, DMSO-d6) δ 14.15 (s, 1H), 8.80-8.64 (m, 2H), 8.06 (s, 3H), 7.38-7.22 (m, 2H), 7.22-7.08 (m, 2H), 6.95-6.81 (m, 1H), 4.51-4.27 (m, 2H), 4.14-3.96 (m, 3H), 3.89-3.67 (m, 12H), 3.13 (s, 1H), 3.14 (m, 2H), 3.07 (dd, J=14.3, 4.9 Hz, 1H), 2.87 (dd, J=14.2, 8.3 Hz, 1H), 2.35-2.25 (m, 1H), 2.21-2.07 (m, 1H), 1.76-1.60 (m, 1H), 1.58-1.39 (m, 2H), 1.17 (t, J=7.1 Hz, 3H), 0.93 (d, J=6.6 Hz, 3H), 0.90 (d, J=6.5 Hz, 3H).

### Example 22: Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate; dihydrochloride (Example compound 22)

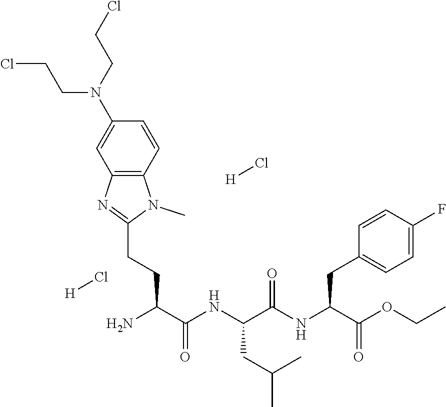

1-(3-Dimethylaminopropyl)-3-ethylcarbodiimide hydrochloride (1.15 eq, 68 mg, 0.355 mmol) was added to a mixture of 4-methylmorpholine (1.15 eq, 0.039 mL, 0.355 mmol), ethyl cyanoglyoxylate-2-oxime (1.00 eq, 44 mg, 0.308 mmol), ethyl (2S)-2-amino-3-(4-fluorophenyl)propanoate (1.15 eq, 74.9 mg, 0.355 mmol) and (2S)-2-[[(2S)-2-amino-4-[5-[bis(2-chloroethyl)amino]-1-methyl-benzimidazol-2-yl]butanoyl]amino]-4-methyl-pentanoic acid (Example compound 20) (1.00 eq, 150 mg, 0.308 mmol) in DMF (2 mL). The reaction mixture was stirred at room temperature for 3h. Water was added, and the mixture was extracted with DCM. The organic phase was washed with a 10% K₂HPO₄ and 10% NaCl, followed by an acidic wash using 0.25M HCl and 10% NaCl, passed through a phase separator cartridge and evaporated. The crude was purified by preparative chromatography to afford ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate; dihydrochloride (Example compound 22) (4 mg, 0.0053 mmol, 2% yield) as a white solid. HPLC purity: 99%; MS (ESI+) m/z=679 [M+H]⁺.

¹H NMR (600 MHz, DMSO-d6) δ 14.34 (br s, 1H), 8.72 (d, J=7.7 Hz, 2H), 8.34 (s, 3H), 7.68 (s, 1H), 7.25 (dd, J=8.3, 5.5 Hz, 2H), 7.01 (t, J=8.7 Hz, 2H), 6.90 (d, J=2.4 Hz, 1H), 4.43 (dq, J=15.5, 7.7 Hz, 2H), 4.05-3.96 (m, 2H), 3.95 (q, J=6.5, 5.8 Hz, 1H), 3.84-3.69 (m, 11H), 3.13-2.98 (m, 4H), 2.93 (dd, J=13.9, 9.0 Hz, 1H), 2.23 (dq, J=13.4, 6.8, 6.1 Hz, 1H), 2.10 (dd, J=15.9, 8.1 Hz, 1H), 1.69 (dp, J=13.5, 6.7 Hz, 1H), 1.47 (t, J=7.4 Hz, 2H), 1.08 (t, J=7.1 Hz, 3H), 0.91 (dd, J=19.1, 6.5 Hz, 6H).

### Example 23: Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate dihydrochloride (Example compound 23)

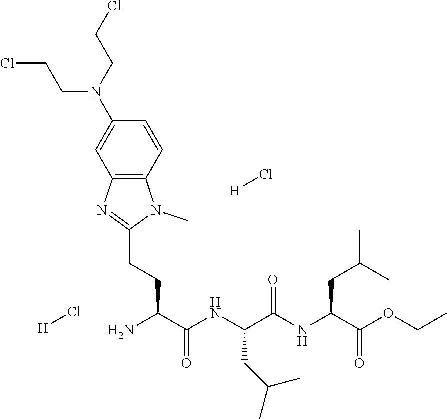 [SMILES: CCOC(=O)[C@H](CC(C)C)NC(=O)[C@H](CC(C)C)NC(=O)[C@@H](N)CCc1nc2cc(N(CCCl)CCCl)ccc2n1C ; Cl ; Cl]

4-Methylmorpholine (1.15 eq, 0.039 mL, 0.355 mmol) was added to a mixture of (2S)-2-[[(2S)-2-amino-4-[5-[bis(2-chloroethyl)amino]-1-methyl-benzimidazol-2-yl]butanoyl]amino]-4-methyl-pentanoic acid (Example compound 20) (1.00 eq, 150 mg, 0.308 mmol), ethyl (2S)-2-amino-4-methyl-pentanoate (1.15 eq, 56 mg, 0.355 mmol), 1-(3-dimethylaminopropyl)-3-ethylcarbodiimide hydrochloride (1.15 eq, 68 mg, 0.355 mmol) and ethyl cyanoglyoxylate-2-oxime (1.00 eq, 44 mg, 0.308 mmol) in DMF (2 mL). The reaction mixture was stirred at room temperature for 3h. Water was added, and the mixture was extracted with DCM. The organic phase was washed with a 10% K₂HPO₄ and 10% NaCl, followed by an acidic wash using 0.25M HCl and 10% NaCl, passed through a phase separator cartridge and evaporated. The crude obtained was purified by preparative chromatography to afford ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate; dihydrochloride (Example compound 23) (5.1 mg, 0.00728 mmol, 2% yield) as a solid. HPLC purity: 95%; MS (ESI+) m/z=627 [M+H]⁺. 1H NMR (600 MHz, DMSO-d6) δ 14.41 (br s, 1H), 8.76 (s, 1H), 8.54 (d, J=7.6 Hz, 1H), 8.24 (d, J=123.3 Hz, 3H), 7.70 (s, 1H), 6.90 (d, J=2.3 Hz, 1H), 6.57 (s, 1H), 4.39 (dt, J=13.0, 6.5 Hz, 1H), 4.27 (ddd, J=10.6, 7.5, 4.9 Hz, 1H), 4.10-4.00 (m, 2H), 3.97 (d, J=7.5 Hz, 1H), 3.91-3.71 (m, 11H), 3.1 (m, 2H), 2.25 (dt, J=13.5, 7.2 Hz, 1H), 2.13 (s, 1H), 1.73 (dt, J=13.4, 6.3 Hz, 1H), 1.64 (dd, J=13.9, 7.0 Hz, 1H), 1.53 (tddd, J=22.7, 14.1, 9.6, 5.1 Hz, 4H), 1.15 (t, J=7.1 Hz, 3H), 0.95 (d, J=6.5 Hz, 3H), 0.92 (d, J=6.5 Hz, 3H), 0.87 (d, J=6.6 Hz, 3H), 0.78 (d, J=6.5 Hz, 3H).

### Example 24: 2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate dihydrochloride (Example compound 24)

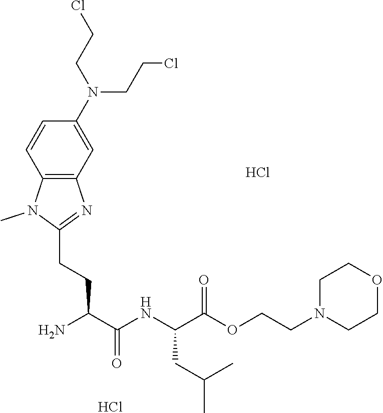 [SMILES: CC(C)C[C@H](NC(=O)[C@@H](N)CCc1nc2cc(N(CCCl)CCCl)ccc2n1C)C(=O)OCCN1CCOCC1 ; Cl ; Cl]

(2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoic acid; dihydrochloride) (1.00 eq, 50 mg, 0.0758 mmol) was suspended in THF (2 mL) and thionyl chloride (5.53 eq, 31 uL, 0.420 mmol) was added. The reaction was stirred at room temperature for 30 minutes and then 4-(2-hydroxyethyl)morpholine (5.00 eq, 0.046 mL, 0.379 mmol) was added. The mixture was stirred at room temperature for 3h. The reaction mixture was filtered and purified by preparative chromatography to afford 2-morpholinoethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 24) (6 mg, 0.0089 mmol, 12% yield) as a solid. HPLC purity: 99%; MS (ESI+) m/z=599 [M+H]⁺.

¹H NMR (600 MHz, DMSO-d6) δ 14.79 (s, 1H), 11.42 (s, 1H), 9.36 (d, J=7.3 Hz, 1H), 8.55 (s, 2H), 7.72 (s, 1H), 7.12 (s, 1H), 6.93 (s, 1H), 4.50 (s, 1H), 4.44 (td, J=13.5, 11.9, 5.2 Hz, 2H), 4.11 (s, 1H), 3.96-3.74 (m, 15H), 3.56 (s, 2H), 3.15 (s, 6H), 2.32 (q, J=7.6 Hz, 2H), 1.74 (dq, J=13.3, 6.6, 6.1 Hz, 1H), 1.63 (dddd, J=18.6, 13.8, 9.1, 4.6 Hz, 2H), 0.91 (dd, J=20.5, 6.5 Hz, 6H).

### Example 25: 2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate dihydrochloride (Example compound 25)

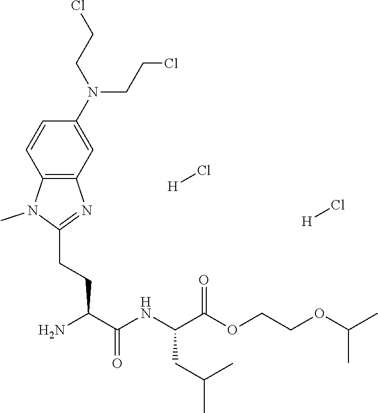 [SMILES: CC(C)C[C@H](NC(=O)[C@@H](N)CCc1nc2cc(N(CCCl)CCCl)ccc2n1C)C(=O)OCCOC(C)C ; Cl ; Cl]

(2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoic acid; dihydrochloride (1.00 eq, 50 mg, 0.0758 mmol) was added in portions to a solution of ethylene glycol monoisopropyl ether (5.00 eq, 0.042 mL, 0.379 mmol) in MeCN (2 mL) and thionyl chloride (5.53 eq, 31 uL, 0.420 mmol) at −5° C. The reaction was allowed to reach room temperature and then heated at 30° C. for 3h. The solvent was removed in vacuum as azeotrope with toluene, and then dried by high-vacuum pump. The crude obtained was purified by preparative chromatography to afford 2-isopropoxyethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 25) (6.2 mg, 0.0096 mmol, 12% yield) as a solid. HPLC purity: 95%; MS (ESI+) m/z=572 [M+H]⁺.

¹H NMR (600 MHz, DMSO-d6) δ 14.77 (s, 1H), 9.25 (d, J=7.0 Hz, 1H), 8.56 (s, 2H), 7.72 (s, 1H), 7.12 (s, 1H), 6.93 (dd, J=10.1, 2.4 Hz, 1H), 6.55 (s, 1H), 4.37-4.28 (m, 1H), 4.23 (dq, J=8.1, 4.4, 3.9 Hz, 1H), 4.09 (ddt, J=13.8, 10.7, 5.5 Hz, 2H), 3.90-3.74 (m, 11H), 3.54 (td, J=8.6, 7.0, 3.6 Hz, 3H), 2.36-2.24 (m, 2H), 1.77 (dd, J=13.9, 6.7 Hz, 1H), 1.60 (ddt, J=28.1, 13.4, 8.4 Hz, 2H), 1.05 (dd, J=18.5, 6.1 Hz, 6H), 0.91 (dd, J=25.5, 6.5 Hz, 6H). One CH₂ signal is hidden under the H₂O signal.

### Example 26: Isopropyl (2S)-2-[[(2S)-2-amino-4-[5-[bis(2-chloroethyl)amino]-1-methyl-benzimidazol-2-yl]butanoyl]amino]-4-methyl-pentanoate dihydrochloride (Example compound 26)

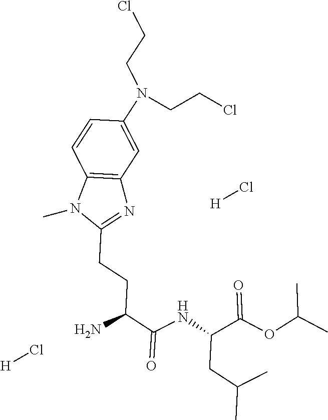 [SMILES: Cl ; CC(C)C[C@H](NC(=O)[C@@H](N)CCc1nc2cc(N(CCCl)CCCl)ccc2n1C)C(=O)OC(C)C ; Cl]

(2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoic acid; dihydrochloride (1.00 eq, 50 mg, 0.0758 mmol) was added in portions to a solution of isopropanol (200 eq, 1.2 mL, 15.2 mmol) and thionyl chloride (5.53 eq, 31 uL, 0.420 mmol) at −5° C. The reaction was allowed to reach room temperature and it was stirred for 3h.

The solvent was evaporated as azeotrope with toluene, and then dried by high-vacuum pump. The crude obtained was purified by preparative chromatography to afford isopropyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 26) (32 mg, 0.053 mmol, 69% yield) as an off-white solid. HPLC purity: 98%; MS (ESI+) m/z=528 [M+H]⁺.

¹H NMR (600 MHz, DMSO-d6) δ 14.79 (s, 1H), 9.20 (d, J=7.0 Hz, 1H), 8.55 (s, 3H), 7.74 (d, J=9.2 Hz, 1H), 7.15 (d, J=9.2 Hz, 1H), 6.93 (s, 1H), 4.90 (p, J=6.3 Hz, 1H), 4.26 (dt, J=11.0, 5.7 Hz, 1H), 4.08 (s, 1H), 3.90 (s, 3H), 3.80 (dt, J=33.7, 6.9 Hz, 8H), 3.37-3.32 (m, 2H), 2.31 (dh, J=22.0, 7.3 Hz, 2H), 1.75 (p, J=6.7 Hz, 1H), 1.62 (ddd, J=15.0, 10.2, 5.1 Hz, 1H), 1.53 (ddd, J=13.9, 9.2, 4.9 Hz, 1H), 1.18 (dd, J=11.4, 6.3 Hz, 6H), 0.90 (dd, J=27.2, 6.5 Hz, 6H).

### Example 27—Methyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 27)

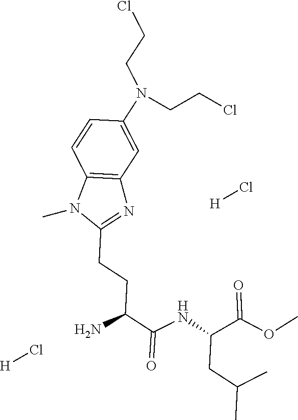

(2S)-2-[[(2S)-2-amino-4-[5-[bis(2-chloroethyl)amino]-1-methyl-benzimidazol-2-yl]butanoyl]amino]-4-methyl-pentanoic acid (Example compound 20) (1.00 eq, 35 mg, 0.06260 mmol) was dissolved in 0.4 mL MeOH (137 eq.). Thionyl chloride (5.72 eq, 30 uL, 0.411 mmol) was added dropwise to the solution at room temperature. The reaction was stirred room temperature for 4.5 hours. The crude reaction mixture was purified by preparative chromatography to give methyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 27) (14 mg, 0.0244 mmol, 33% yield) as an off-white. HPLC purity: 97%; MS (ESI+) m/z=500 [M+H]⁺.

¹H NMR (600 MHz, DMSO-d6) δ 14.86 (s, 1H), 9.32 (d, J=7.1 Hz, 1H), 8.61 (s, 3H), 7.74 (d, J=9.2 Hz, 1H), 7.15 (d, J=9.2 Hz, 1H), 6.94 (s, 1H), 4.33 (dt, J=11.1, 5.5 Hz, 1H), 4.10 (s, 1H), 3.91 (s, 3H), 3.80 (dt, J=33.0, 6.9 Hz, 8H), 3.65 (s, 3H), 3.35 (h, J=10.1, 8.5 Hz, 2H), 2.33 (ddq, J=30.2, 15.1, 7.5 Hz, 2H), 1.75 (p, J=6.7 Hz, 1H), 1.65 (td, J=12.2, 10.3, 5.1 Hz, 1H), 1.54 (ddd, J=14.2, 9.6, 4.7 Hz, 1H), 0.90 (dd, J=23.3, 6.5 Hz, 6H).

### Example 28—3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 28)

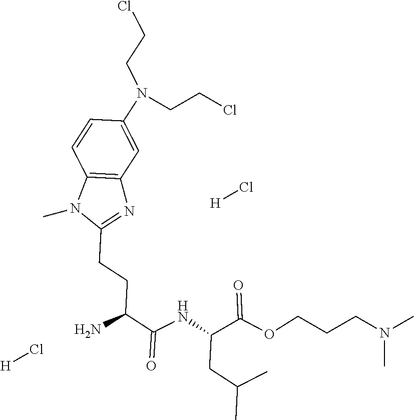

Ethyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 1) (1.00 eq, 50 mg, 0.0851 mmol) was dissolved in THF (1 mL). HCl (4M in EtOAc) (10.0 eq, 0.21 mL, 0.851 mmol) was added followed by 3-dimethylamino-1-propanol (10.0 eq, 0.10 mL, 0.851 mmol). The reaction was stirred at 80° C. for 4 hours. The mixture was filtered and purified by preparative chromatography to afford 3-(dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 28) (2 mg, 0.0031 mmol, 3% yield) as an off-white solid. HPLC purity: 95%; MS (ESI+) m/z=571 [M+H]⁺.

¹H NMR (600 MHz, DMSO-d6) b 15.9 (br s, 1H), 10.7 (br s, 1H), 9.43 (d, J=7.1 Hz, 1H), 8.55 (d, J=73.7 Hz, 3H), 7.79 (m, 1H), 6.93 (s, 1H), 4.34 (s, 1H), 4.18-4.12 (m, 2H), 3.76 (s, 11H), 3.09 (s, 1H), 2.73 (d, J=20.6 Hz, 6H), 2.34 (s, 1H), 2.01 (q, J=7.8, 7.3 Hz, 2H), 1.81-1.52 (m, 4H), 1.23 (s, 4H), 0.97-0.89 (m, 6H).

### Example 29—(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 29)

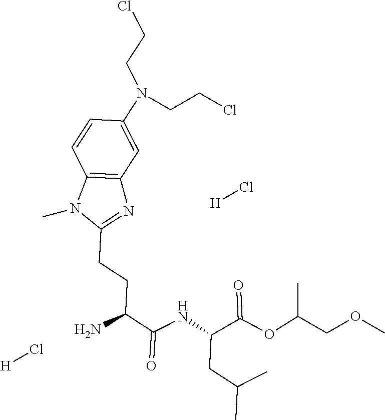

(2S)-2-[[(2S)-2-amino-4-[5-[bis(2-chloroethyl)amino]-1-methyl-benzimidazol-2-yl]butanoyl]amino]-4-methyl-pentanoic acid; dihydrochloride (Example compound 20) (1.0 eq, 36 mg, 0.0644 mmol) was dissolved in 1-methoxy-2-propanol (55 eq, 0.40 mL, 4.07 mmol) and thionyl chloride (5.6 eq, 30 uL, 0.411 mmol) was added dropwise at room temperature. The reaction was stirred room temperature for 1 hr and then heated at 40° C. for 21 hours. The crude reaction mixture was purified by preparative chromatography to give (2-methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate; dihydrochloride (Example compound 29) (2.7 mg 0.0043 mmol, 6% yield) as an off-white solid. HPLC purity: 99%; MS (ESI+) m/z=558 [M+H]⁺.

¹H NMR (600 MHz, DMSO-d6) δ 14.88 (s, 1H), 9.30 (p, J=6.1 Hz, 1H), 8.60 (s, 3H), 7.75 (d, J=9.2 Hz, 1H), 7.15 (d, J=9.1 Hz, 1H), 6.94 (s, 1H), 4.96 (dtd, J=16.3, 6.5, 4.0 Hz, 1H), 4.28 (qd, J=11.2, 10.4, 6.1 Hz, 1H), 4.13-4.05 (m, 1H), 3.91 (s, 3H), 3.83 (t, J=6.8 Hz, 4H), 3.78 (t, J=6.5 Hz, 4H), 3.40-3.33 (m, 4H), 3.22 (d, J=13.7 Hz, 3H), 2.34 (dd, J=18.1, 9.2 Hz, 2H), 1.77 (td, J=9.9, 9.3, 4.6 Hz, 1H), 1.62 (ddd, J=15.1, 12.3, 7.7 Hz, 1H), 1.53 (ddt, J=13.8, 9.6, 4.8 Hz, 1H), 1.14 (dd, J=11.9, 6.5 Hz, 3H), 0.90 (dd, J=26.5, 6.6 Hz, 6H).

### Example 30—(2S)-2-[[(2S)-2-Amino-4-[5-[bis(2-chloroethyl)amino]-1-methyl-benzimidazol-2-yl]butanoyl]amino]-N,N,4-trimethyl-pentanamide; dihydrochloride (Example compound 30)

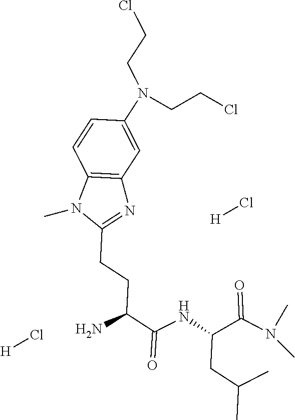

3-(3-Dimethylaminopropyl)-1-ethyl-carbodiimide hydrochloride (1.15 eq, 59 mg, 0.308 mmol) was added to a mixture of (2S)-2-[[(2S)-2-amino-4-[5-[bis(2-chloroethyl)amino]-1-methyl-benzimidazol-2-yl]butanoyl]amino]-4-methyl-pentanoic acid; dihydrochloride (Example compound 20) (1.00 eq, 150 mg, 0.268 mmol), dimethylamine hydrochloride (1.15 eq, 25 mg, 0.308 mmol), ethyl (2E)-cyano(hydroxyimino)acetate (1.00 eq, 38 mg, 0.268 mmol) and N-methylmorpholine (2.00 eq, 0.059 mL, 0.536 mmol) in DMF (1 mL). The reaction was stirred at room temperature for 3h. Water and DCM were added, and the mixture was extracted with 10% K₂HPO₄ and 10% NaCl twice, followed by an acidic wash using 0.25M HCl and 10% NaCl. The organic phase was concentrated, and the crude obtained was purified by preparative chromatography to afford (2S)-2-[[(2S)-2-amino-4-[5-[bis(2-chloroethyl)amino]-1-methyl-benzimidazol-2-yl]butanoyl]amino]-N,N,4-trimethyl-pentanamide; dihydrochloride (Example compound 30) (13 mg, 0.022 mmol, 8% yield) as solid. HPLC purity: 95%; MS (ESI+) m/z=513 [M+H]⁺. 1H NMR (600 MHz, DMSO-d6) δ 14.63 (s, 1H), 8.87 (s, 1H), 8.51 (s, 3H), 7.73 (s, 1H), 7.14 (s, 1H), 6.91 (d, J=2.4 Hz, 1H), 4.72 (ddd, J=11.0, 7.5, 3.6 Hz, 1H), 4.00 (s, 1H), 3.89 (s, 3H), 3.82 (d, J=6.7 Hz, 4H), 3.77 (t, J=6.4 Hz, 4H), 3.3 (m, 2H), 3.04 (s, 3H), 2.84 (s, 3H), 2.39-2.30 (m, 1H), 2.19 (dq, J=14.3, 7.3 Hz, 1H), 1.75-1.67 (m, 1H), 1.50 (ddd, J=14.7, 10.8, 4.2 Hz, 1H), 1.39 (ddd, J=13.7, 9.8, 3.6 Hz, 1H), 0.92 (t, J=6.1 Hz, 6H).

### Example 31—(2S)-2-[[(2S)-2-amino-4-[5-[bis(2-chloroethyl)amino]-1-methyl-benzimidazol-2-yl]butanoyl]amino]-N,4-dimethyl-pentanamide; dihydrochloride (Example compound 31)

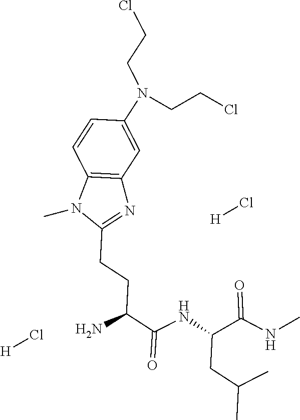

Example 31 was synthesized according to Scheme 9 below:

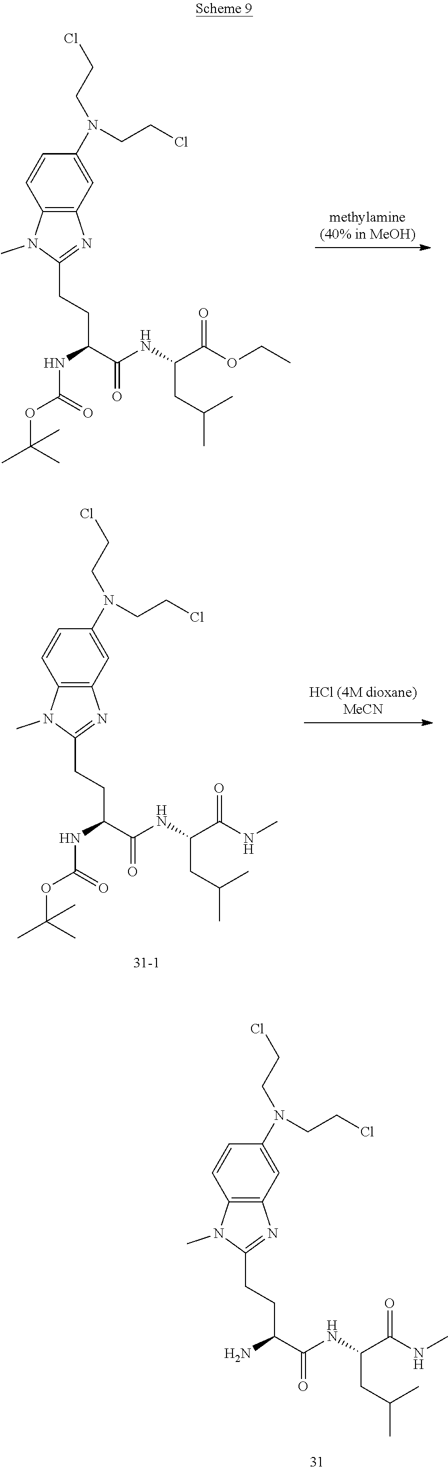

### tert-Butyl N-[(1S)-3-[5-[bis(2-chloroethyl)amino]-1-methyl-benzimidazol-2-yl]-1-[[(1S)-3-methyl-1-(methylcarbamoyl)butyl]carbamoyl]propyl]carbamate; dihydrochloride (compound 31-1)

Ethyl (2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate (step 4, in the synthetic route towards Example compound 1) (50 mg, 0.0814 mmol) in methylamine (40% in MeOH) (63 mg, 0.814 mmol) was stirred for 20 minutes at 80° C. under microwave irradiation in a sealed tube. The solvent was removed under reduced pressure and the crude was purified by preparative chromatography to afford tert-butyl N-[(1S)-3-[5-[bis(2-chloroethyl)amino]-1-methyl-benzimidazol-2-yl]-1-[[(1S)-3-methyl-1-(methylcarbamoyl)butyl]carbamoyl]propyl]carbamate; dihydrochloride (35 mg, 0.0586 mmol, 64% yield) as a white solid. HPLC purity: 99%; MS (ESI+) m/z=599 [M+H]⁺.

### (2S)-2-[[(2S)-2-amino-4-[5-[bis(2-chloroethyl)amino]-1-methyl-benzimidazol-2-yl]butanoyl]amino]-N,4-dimethyl-pentanamide; dihydrochloride (Example compound 31)

tert-Butyl N-[(1S)-3-[5-[bis(2-chloroethyl)amino]-1-methyl-benzimidazol-2-yl]-1-[[(1S)-3-methyl-1-(methylcarbamoyl)butyl]carbamoyl]propyl]carbamate (35 mg, 0.0586 mmol) was dissolved in MeCN (1 mL). Hydrochloric acid (4M in dioxane) (0.15 mL, 0.586 mmol) was added and the mixture was stirred at room temperature for 3 hours. The reaction mixture was purified by preparative chromatography to afford (2S)-2-[[(2S)-2-amino-4-[5-[bis(2-chloroethyl)amino]-1-methyl-benzimidazol-2-yl]butanoyl]amino]-N,4-dimethyl-pentanamide; dihydrochloride (Example compound 31) (14 mg, 0.024 mmol, 40% yield) as an off-white solid. HPLC purity: 95%; MS (ESI+) m/z=499 [M+H]⁺.

¹H NMR (600 MHz, DMSO-d6) δ 14.62 (s, 1H), 8.79 (s, 1H), 8.48 (s, 3H), 8.18 (q, J=4.6 Hz, 1H), 7.74 (s, 1H), 7.15 (s, 1H), 6.91 (d, J=2.4 Hz, 1H), 4.29 (ddd, J=10.0, 7.6, 5.0 Hz, 1H), 3.98 (s, 1H), 3.90 (s, 3H), 3.82 (s, 4H), 3.77 (t, J=6.4 Hz, 4H), 3.3 (m, 2H), 2.59 (d, J=4.6 Hz, 3H), 2.33 (s, 1H), 2.21 (dq, J=14.0, 7.2 Hz, 1H), 1.66 (dt, J=13.3, 6.5 Hz, 1H), 1.52 (ddd, J=14.7, 10.0, 5.1 Hz, 1H), 1.43 (ddd, J=13.8, 9.0, 5.1 Hz, 1H), 0.90 (dd, J=15.6, 6.6 Hz, 6H).

### Example 32—Ethyl (2S)-2-[[(2S)-4-[5-[bis(2-chloroethyl)amino]-1-methyl-benzimidazol-2-yl]-2-(methylamino)butanoyl]amino]-4-methyl-pentanoate; dihydrochloride (Example compound 32)

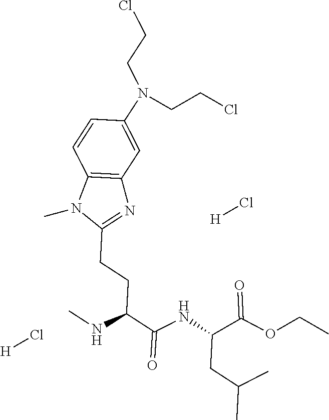

Example 32 was synthesized according to Scheme 10 below:

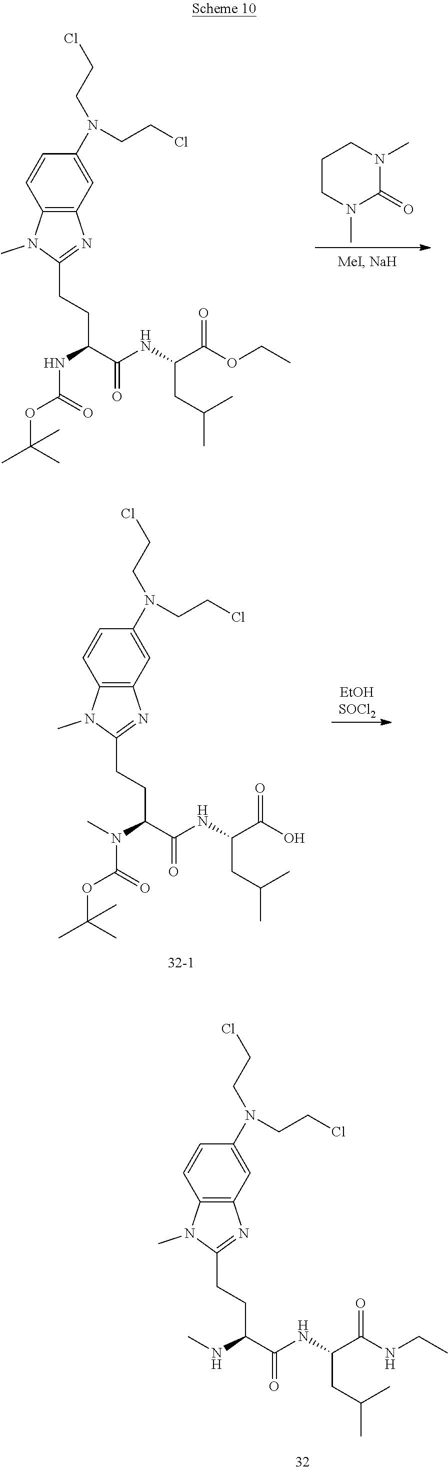

### (2S)-2-[[(2S)-4-[5-[bis(2-Chloroethyl)amino]-1-methyl-benzimidazol-2-yl]-2-[tert-butoxycarbonyl(methyl)amino]butanoyl]amino]-4-methyl-pentanoic acid (compound 32-1)

To a solution of ethyl (2S)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate (step 4, in the synthetic route towards Example compound 1) (300 mg, 0.488 mmol) in dry THF (3 mL) was added sodium hydride (50%; 59 mg, 1.46 mmol). The mixture was stirred at room temperature until the end of gas evolution. 1,3-Dimethyl-3,4,5,6-tetrahydro-2(1H)-pyrimidinone (0.12 mL, 1.03 mmol) was added to the reaction mixture followed by iodomethane (0.27 mL, 4.39 mmol). The reaction was stirred at room temperature overnight. The reaction was quenched carefully with water and purified by preparative chromatography to afford (2S)-2-[[(2S)-4-[5-[bis(2-chloroethyl)amino]-1-methyl-benzimidazol-2-yl]-2-[tert-butoxycarbonyl(methyl)amino]butanoyl]amino]-4-methyl-pentanoic acid (105 mg, 0.175 mmol, 36% yield) as a white solid. HPLC purity: 99%; MS (ESI+) m/z=600 [M+H]⁺.

### Ethyl (2S)-2-[[(2S)-4-[5-[bis(2-chloroethyl)amino]-1-methyl-benzimidazol-2-yl]-2-(methylamino)butanoyl]amino]-4-methyl-pentanoate; dihydrochloride (Example compound 32)

(2S)-2-[[(2S)-4-[5-[bis(2-Chloroethyl)amino]-1-methyl-benzimidazol-2-yl]-2-[tert-butoxycarbonyl(methyl)amino]butanoyl]amino]-4-methyl-pentanoic acid (42 mg, 0.0699 mmol) was added in portions to a solution of ethanol (2 mL) and thionyl chloride (31 uL, 0.420 mmol) at −5° C. The reaction was stirred at room temperature for 5 minutes and then refluxed for 2h. The reaction was allowed to reach temperature and the solvent was evaporated under vacuum as an azeotrope with toluene, and then dried by high-vacuum pump. The crude was purified by preparative chromatography to afford ethyl (2S)-2-[[(2S)-4-[5-[bis(2-chloroethyl)amino]-1-methyl-benzimidazol-2-yl]-2-(methylamino)butanoyl]amino]-4-methyl-pentanoate; dihydrochloride (Example compound 32) (25 mg, 0.041 mmol, 59% yield) as an off-white solid. HPLC purity: 99%; MS (ESI+) m/z=528 [M+H]⁺.

¹H NMR (600 MHz, DMSO-d6) δ 14.84 (s, 1H), 9.43 (d, J=7.0 Hz, 3H), 7.73 (d, J=9.2 Hz, 1H), 7.14 (d, J=9.4 Hz, 1H), 6.93 (d, J=2.5 Hz, 1H), 4.35 (ddd, J=11.0, 7.0, 4.7 Hz, 1H), 4.12 (p, J=7.0 Hz, 2H), 4.04 (dd, J=8.0, 4.8 Hz, 1H), 3.90 (s, 3H), 3.83 (t, J=6.8 Hz, 4H), 3.77 (t, J=6.5 Hz, 4H), 3.30 (t, J=8.4 Hz, 2H), 2.55 (s, 3H), 2.47 (dd, J=8.5, 4.9 Hz, 1H), 2.28 (dq, J=14.8, 7.9 Hz, 1H), 1.69 (dddd, J=39.0, 15.1, 9.7, 5.6 Hz, 2H), 1.57 (ddd, J=13.8, 9.2, 4.8 Hz, 1H), 1.19 (t, J=7.1 Hz, 3H), 0.91 (dd, J=26.6, 6.5 Hz, 6H).

### Example 33: (2S)-2-[[(2S)-4-[5-[bis(2-chloroethyl)amino]-1-methyl-benzimidazol-2-yl]-2-(methylamino)butanoyl]amino]-4-methyl-pentanoic acid; dihydrochloride (Example compound 33)

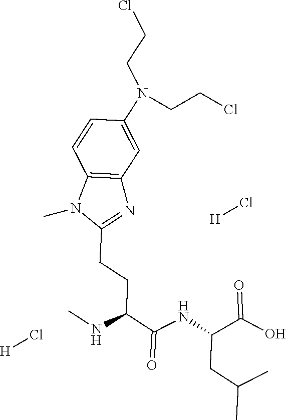

(2S)-2-[[(2S)-4-[5-[bis(2-Chloroethyl)amino]-1-methyl-benzimidazol-2-yl]-2-[tert-butoxycarbonyl(methyl)amino]butanoyl]amino]-4-methyl-pentanoic acid (compound 32-1) (12 mg, 0.0200 mmol) was dissolved in ethanol (1 mL). HCl (4M in dioxane) (50 uL, 0.200 mmol) was added and the reaction was stirred at room temperature for 3h. The reaction mixture was filtrated and purified by preparative chromatography to afford (2S)-2-[[(2S)-4-[5-[bis(2-chloroethyl)amino]-1-methyl-benzimidazol-2-yl]-2-(methylamino)butanoyl]amino]-4-methyl-pentanoic acid; dihydrochloride (Example compound 33) (4 mg, 0.0069 mmol, 34% yield) as an off-white solid. HPLC purity: 97%; MS (ESI+) m/z=500 [M+H]⁺.

¹H NMR (600 MHz, DMSO-d6) δ 14.43 (s, 1H), 13.03 (s, 1H), 9.13 (s, 2H), 7.63 (s, 1H), 7.16-6.98 (m, 1H), 6.92 (d, J=2.4 Hz, 1H), 4.34 (q, J=7.5 Hz, 1H), 3.95 (dd, J=7.8, 4.7 Hz, 1H), 3.87-3.72 (m, 11H), 3.14 (s, 3H), 2.57 (s, 3H), 2.44-2.36 (m, 1H), 2.21 (ddt, J=19.1, 13.7, 5.8 Hz, 1H), 1.70 (dp, J=13.2, 6.6 Hz, 1H), 1.62 (t, J=7.4 Hz, 2H), 0.92 (dd, J=28.5, 6.5 Hz, 6H).

### Example 34: Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate; hydrochloride (Example compound 34)

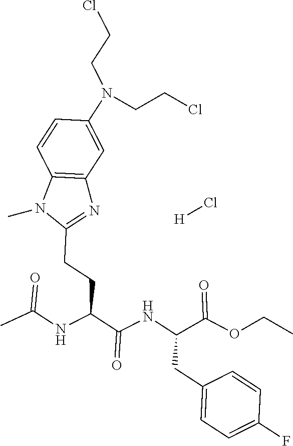 [SMILES: CCOC(=O)[C@H](Cc1ccc(F)cc1)NC(=O)[C@H](CCc1nc2cc(N(CCCl)CCCl)ccc2n1C)NC(C)=O ; Cl]

Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate; hydrochloride (Example compound 2) (15 mg, 0.0233 mmol) was dissolved in acetonitrile (1 mL). N,N-Diisopropylethylamine (0.00553 mL, 0.0317 mmol) was added followed by acetyl chloride (0.00227 mL, 0.0318 mmol) and the reaction was stirred at room temperature for 3h. The reaction mixture was purified by preparative chromatography to afford ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate; hydrochloride (Example compound 34) (7 mg, 0.0108 mmol, 41% yield) as an off-white solid. HPLC purity: 99%; MS (ESI+) m/z=608 [M+H]⁺.

¹H NMR (600 MHz, DMSO-d6) δ 14.20 (s, 1H), 8.49 (d, J=7.2 Hz, 1H), 8.18 (d, J=8.3 Hz, 1H), 7.70 (d, J=9.1 Hz, 1H), 7.29-7.19 (m, 2H), 7.14-7.04 (m, 3H), 6.86 (d, J=2.4 Hz, 1H), 4.40 (dtd, J=20.2, 8.2, 6.1 Hz, 2H), 4.00 (qd, J=7.2, 2.2 Hz, 2H), 3.84-3.74 (m, 11H), 3.05 (q, J=9.3, 8.4 Hz, 2H), 3.00-2.88 (m, 2H), 2.09 (ddd, J=13.9, 8.2, 4.3 Hz, 1H), 2.04-1.94 (m, 1H), 1.76 (s, 3H), 1.06 (t, J=7.1 Hz, 3H).

### Example 35: Ethyl (2S)-2-[[(2S)-2-acetamido-3-[5-[bis(2-chloroethyl)amino]-1-methyl-benzimidazol-2-yl]propanoyl]amino]-4-methyl-pentanoate; hydrochloride (Example compound 35)

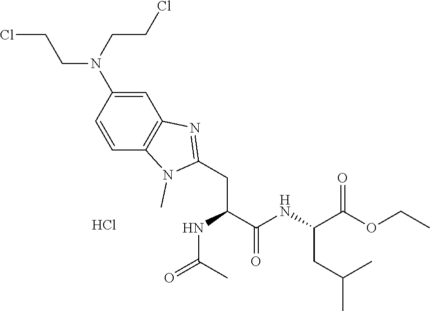 [SMILES: CCOC(=O)[C@H](CC(C)C)NC(=O)[C@H](Cc1nc2cc(N(CCCl)CCCl)ccc2n1C)NC(C)=O ; Cl]

Ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4) (11 mg, 0.0182 mmol) was dissolved in acetonitrile (1 mL). N,N-Diisopropylethylamine (0.00381 mL, 0.0219 mmol) was added followed by acetyl chloride (0.00156 mL, 0.0219 mmol) and the reaction was stirred at room temperature for 3h. The reaction mixture was purified by preparative chromatography to afford ethyl (2S)-2-[[(2S)-2-acetamido-3-[5-[bis(2-chloroethyl)amino]-1-methyl-benzimidazol-2-yl]propanoyl]amino]-4-methyl-pentanoate; hydrochloride (Example compound 35) (8 mg, 0.0138 mmol, 76% yield) as a white solid. HPLC purity: 99%; MS (ESI+) m/z=542 [M+H]⁺.

¹H NMR (600 MHz, DMSO-d6) δ 14.16 (s, 1H), 8.56 (d, J=7.7 Hz, 1H), 8.41 (d, J=8.2 Hz, 1H), 7.72 (d, J=9.2 Hz, 1H), 7.12 (dd, J=9.2, 2.4 Hz, 1H), 6.87 (d, J=2.3 Hz, 1H), 4.83 (td, J=8.0, 6.5 Hz, 1H), 4.26 (ddd, J=9.8, 7.6, 5.2 Hz, 1H), 4.00 (qd, J=7.2, 1.2 Hz, 2H), 3.91 (s, 3H), 3.81 (dd, J=8.9, 6.4 Hz, 4H), 3.76 (t, J=6.1 Hz, 4H), 3.29 (dd, J=15.0, 7.8 Hz, 2H), 1.80 (s, 3H), 1.61-1.49 (m, 2H), 1.46 (ddd, J=13.7, 8.9, 5.1 Hz, 1H), 1.11 (t, J=7.1 Hz, 3H), 0.83 (dd, J=25.2, 6.4 Hz, 6H).

### Example 36: Ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate; hydrochloride (Example compound 36)

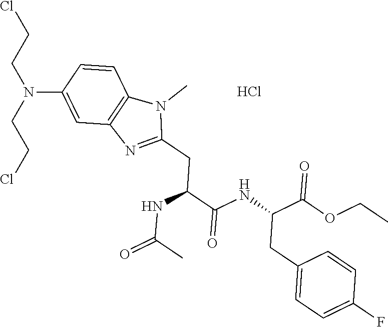 [SMILES: CCOC(=O)[C@H](Cc1ccc(F)cc1)NC(=O)[C@H](Cc1nc2cc(N(CCCl)CCCl)ccc2n1C)NC(C)=O ; Cl]

Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate; dihydrochloride (Example compound 5) (15 mg, 0.0228 mmol) was dissolved in acetonitrile (1 mL). N,N-Diisopropylethylamine (0.00476 mL, 0.0273 mmol) was added followed by acetyl chloride (0.00195 mL, 0.0273 mmol) and the reaction was stirred at room temperature for 3h. The reaction mixture was purified by preparative chromatography to afford ethyl (2S)-2-[[(2S)-2-acetamido-3-[5-[bis(2-chloroethyl)amino]-1-methyl-benzimidazol-2-yl]propanoyl]amino]-3-(4-fluorophenyl)propanoate; hydrochloride (Example compound 36) (10.6 mg, 0.0168 mmol, 71% yield) as a white solid. HPLC purity: 97%; MS (ESI+) m/z=594 [M+H]⁺.

¹H NMR (600 MHz, DMSO-d6) δ 14.18 (s, 1H), 8.62 (d, J=7.6 Hz, 1H), 8.34 (d, J=8.2 Hz, 1H), 7.71 (d, J=9.2 Hz, 1H), 7.24-7.18 (m, 2H), 7.12 (dd, J=9.2, 2.4 Hz, 1H), 7.10-7.03 (m, 2H), 6.86 (d, J=2.4 Hz, 1H), 4.83 (td, J=8.0, 6.5 Hz, 1H), 4.45 (td, J=8.0, 6.1 Hz, 1H), 3.97 (qd, J=7.1, 2.1 Hz, 2H), 3.88 (s, 3H), 3.85-3.79 (m, 4H), 3.75 (t, J=7.1 Hz, 4H), 3.25 (dd, J=15.0, 7.9 Hz, 2H), 3.01-2.89 (m, 2H), 1.79 (s, 3H), 1.04 (t, J=7.1 Hz, 3H).

### Example 37: (2S)-2-[[(2S)-2-Amino-4-methyl-pentanoyl]amino]-4-[5-[bis(2-chloroethyl)amino]-1-methyl-benzimidazol-2-yl]butanoic acid; dihydrochloride (Example compound 37)

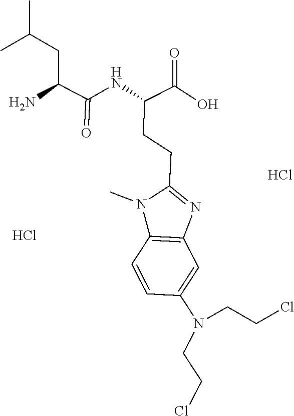 [SMILES: CC(C)C[C@H](N)C(=O)N[C@@H](CCc1nc2cc(N(CCCl)CCCl)ccc2n1C)C(=O)O ; Cl ; Cl]

To a solution of ethyl (2S)-2-[[(2S)-2-amino-4-methyl-pentanoyl]amino]-4-[5-[bis(2-chloroethyl)amino]-1-methyl-benzimidazol-2-yl]butanoate; dihydrochloride (Example compound 7) (6 mg, 0.0102 mmol) in THF:MeOH (2:1, 1 mL) was added a solution of lithium hydroxide monohydrate in water (0.5 M, 0.0238 mL). The mixture was stirred overnight at room temperature, and then filtered and purified by preparative chromatography to afford (2S)-2-[[(2S)-2-amino-4-methyl-pentanoyl]amino]-4-[5-[bis(2-chloroethyl)amino]-1-methyl-benzimidazol-2-yl]butanoic acid; dihydrochloride (Example compound 37) (1 mg, 0.00177 mmol, 16% yield) as a white solid. HPLC purity: 99%; MS (ESI+) m/z=486 [M+H]⁺.

¹H NMR (600 MHz, DMSO-d6) δ 14.50 (s, 1H), 13.04 (s, 1H), 9.14 (d, J=8.0 Hz, 1H), 8.38 (d, J=5.4 Hz, 3H), 7.73 (d, J=9.2 Hz, 1H), 7.13 (dd, J=9.3, 2.4 Hz, 1H), 6.88 (d, J=2.4 Hz, 1H), 4.37 (td, J=8.8, 4.0 Hz, 1H), 3.91 (s, 3H), 3.86 (q, J=6.6 Hz, 1H), 3.81 (t, J=6.8 Hz, 4H), 3.76 (t, J=6.6 Hz, 4H), 3.32-3.22 (m, 2H), 2.36 (t, J=8.8 Hz, 1H), 2.12 (ddt, J=14.0, 9.2, 4.7 Hz, 1H), 1.69 (dq, J=13.3, 6.6 Hz, 1H), 1.59 (dt, J=13.9, 7.1 Hz, 1H), 1.51 (dt, J=14.2, 7.4 Hz, 1H), 0.90 (dd, J=22.4, 6.5 Hz, 6H).

### Example 38: Ethyl (2S)-2-amino-4-[5-[bis(2-chloroethyl)amino]-1-methyl-benzimidazol-2-yl]butanoate; dihydrochloride (Example compound 38)

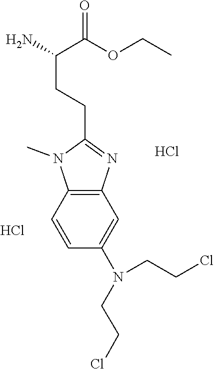 [SMILES: Cl ; CCOC(=O)[C@@H](N)CCc1nc2cc(N(CCCl)CCCl)ccc2n1C ; Cl]

To a solution of ethyl (2S)-4-[5-[bis(2-chloroethyl)amino]-1-methyl-benzimidazol-2-yl]-2-(tert-butoxycarbonylamino)butanoate (synthesised according to Scheme 6 using ethyl (2S)-2-(tert-butoxycarbonylamino)-4-(1-methyl-5-nitro-benzimidazol-2yl)butanoate depicted in Scheme 1 as starting material) (559 mg, 1.06 mmol, 95% purity) in EtOH (5 mL) was added hydrogen chloride in dioxane (4.00 mol/L, 2.65 mL, 10.6 mmol) and the reaction mixture was stirred at 25° C. for 3h. The solvents were evaporated under reduced pressure to give the crude product, which was purified by preparative chromatography to afford ethyl (2S)-2-amino-4-[5-[bis(2-chloroethyl)amino]-1-methyl-benzimidazol-2-yl]butanoate; dihydrochloride (Example compound 38) (416 mg, 0.877 mmol, 83% yield) as a white solid. HPLC purity: 99%; MS (ESI+) m/z=401 [M+H]⁺.

¹H NMR (600 MHz, DMSO-d6) δ 14.81 (s, 1H), 8.86 (s, 3H), 7.85-7.68 (m, 1H), 7.15 (d, J=9.6 Hz, 1H), 6.92 (d, J=5.0 Hz, 1H), 4.27 (s, 1H), 4.21 (t, J=6.5 Hz, 2H), 3.93 (t, J=4.4 Hz, 3H), 3.84 (q, J=6.0 Hz, 4H), 3.78 (t, J=5.8 Hz, 4H), 3.36 (dq, J=16.9, 6.1 Hz, 2H), 2.44-2.31 (m, 2H), 1.25 (q, J=6.3 Hz, 3H).

### Example 39: (2S)-2-[[(2S)-2-Amino-4-[5-[bis(2-chloroethyl)amino]-1-methyl-benzimidazol-2-yl]butanoyl]amino]-3-(4-fluorophenyl)propanoic acid; dihydrochloride (Example compound 39)

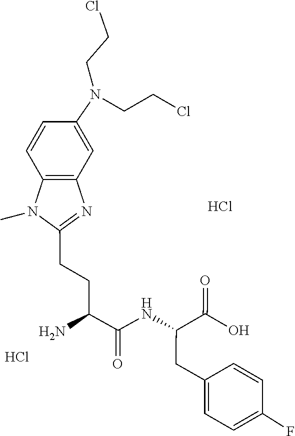 [SMILES: Cn1c(CC[C@H](N)C(=O)N[C@@H](Cc2ccc(F)cc2)C(=O)O)nc2cc(N(CCCl)CCCl)ccc21 ; Cl ; Cl]

Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate; dihydrochloride (Example compound 2) (10.2 mg, 0.015 mmol) was dissolved in HCl (4M in dioxane, 0.0758 mL) and stirred at room temperature for 48 hours. The reaction mixture was purified by preparative chromatography to afford (2S)-2-[[(2S)-2-amino-4-[5-[bis(2-chloroethyl)amino]-1-methyl-benzimidazol-2-yl]butanoyl]amino]-3-(4-fluorophenyl)propanoic acid; dihydrochloride butanoate (Example compound 39) (2.3 mg, 0.00348 mmol, 25% yield) as a white solid. HPLC purity: 99%; MS (ESI+) m/z=538 [M+H]⁺.

¹H NMR (600 MHz, DMSO-d6) δ 14.74 (s, 1H), 13.10 (s, 1H), 9.23 (s, 1H), 8.54 (s, 3H), 7.73 (d, J=9.3 Hz, 1H), 7.36 (ddd, J=8.1, 5.6, 2.1 Hz, 2H), 7.16-7.07 (m, 3H), 6.91 (d, J=2.3 Hz, 1H), 4.47 (td, J=8.4, 4.8 Hz, 1H), 4.04 (q, J=5.9 Hz, 1H), 3.87 (d, J=1.5 Hz, 3H), 3.82 (d, J=6.9 Hz, 4H), 3.76 (d, J=6.2 Hz, 4H), 3.32 (s, 2H), 3.10 (dd, J=14.1, 4.7 Hz, 1H), 2.98 (dd, J=14.1, 8.9 Hz, 1H), 2.38-2.22 (m, 2H).

### Example 40: (2S)-2-[[(2S)-2-Amino-4-[5-[bis(2-chloroethyl)amino]-1-phenyl-benzimidazol-2-yl]butanoyl]amino]-4-methyl-pentanoic acid; dihydrochloride (Example compound 40)

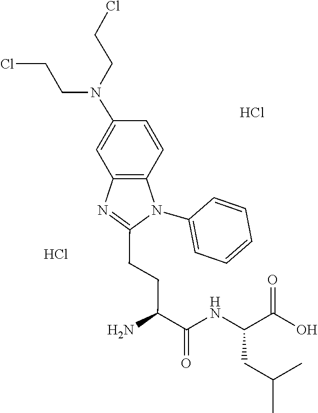 [SMILES: CC(C)C[C@H](NC(=O)[C@@H](N)CCc1nc2cc(N(CCCl)CCCl)ccc2n1-c1ccccc1)C(=O)O ; Cl ; Cl]

Ethyl (2S)-2-[[(2S)-4-[5-[bis(2-chloroethyl)amino]-1-phenyl-benzimidazol-2-yl]-2-(tert-butoxycarbonylamino)butanoyl]amino]-4-methyl-pentanoate (compound 13-11) (90.0 mg, 0.000133 mol) was dissolved in 1,4-dioxane (5.0 mL) and then water (1.0 mL) and LiOH·H₂O (3 eq, 16 mg), were added. The mixture was stirred at 50° C. for 2h.

Water (10 mL) and DCM (10 mL) were added, and the pH was adjusted to 5-6 with HCl (aq.). The different phases of the mixture were separated, and the organic phase was washed with water and dried over MgSO₄. The solvents were removed under vacuum. The residue was redissolved in DCM (2 mL) and then HCl (4M in 1,4-dioxane, 1.0 mL) was added, and the solution stirred for 30 minutes at rt. The mixture was evaporated to dryness to afford (2S)-2-[[(2S)-2-Amino-4-[5-[bis(2-chloroethyl)amino]-1-phenyl-benzimidazol-2-yl]butanoyl]amino]-4-methyl-pentanoic acid; dihydrochloride (Example compound 40) (70 mg, 0.113 mmol, 90% yield) as a solid. HPLC purity: 89%; MS (ESI+) m/z=548 [M+H]⁺.

### Example 41: (2S)-2-Amino-4-[5-[bis(2-chloroethyl)amino]-1-phenyl-benzimidazol-2-yl]butanoic acid (Example compound 41)

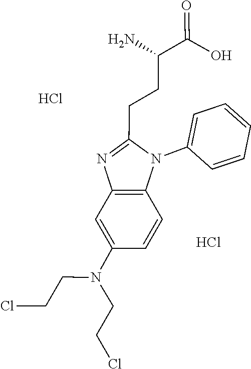 [SMILES: N[C@@H](CCc1nc2cc(N(CCCl)CCCl)ccc2n1-c1ccccc1)C(=O)O ; Cl ; Cl]

Example 41 was synthesized according to Scheme 11 below:

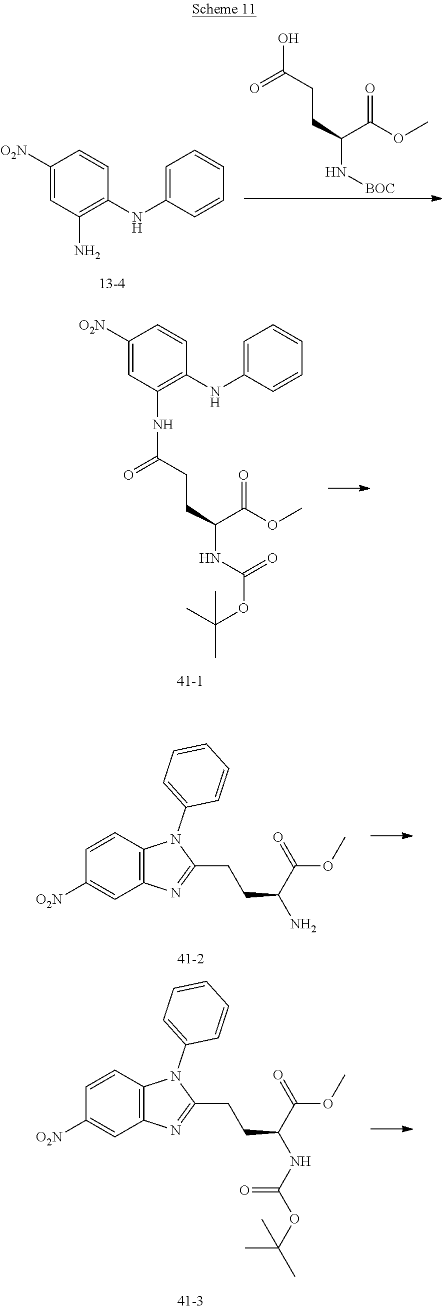 [SMILES: Nc1cc([N+](=O)[O-])ccc1Nc1ccccc1 ; COC(=O)[C@H](CCC(=O)O)NC(=O)OC(C)(C)C ; COC(=O)[C@H](CCC(=O)Nc1cc([N+](=O)[O-])ccc1Nc1ccccc1)NC(=O)OC(C)(C)C ; COC(=O)[C@@H](N)CCc1nc2cc([N+](=O)[O-])ccc2n1-c1ccccc1 ; COC(=O)[C@H](CCc1nc2cc([N+](=O)[O-])ccc2n1-c1ccccc1)NC(=O)OC(C)(C)C]

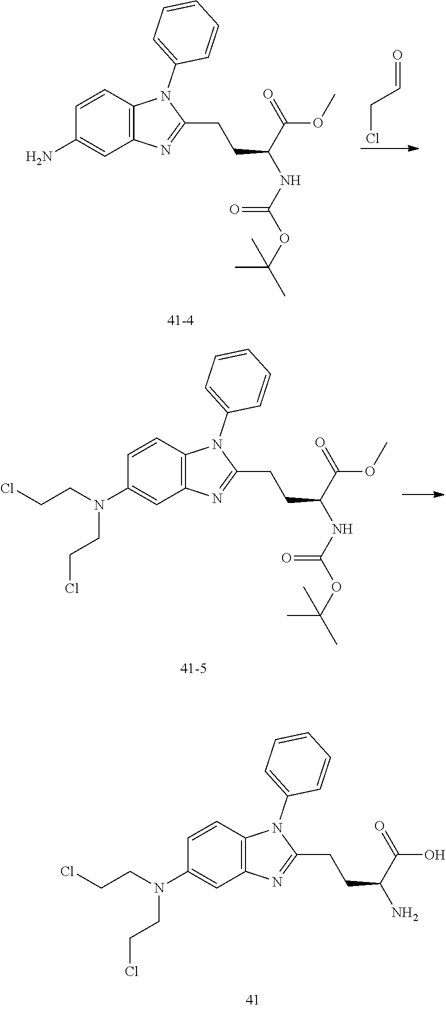

### Methyl (2S)-5-(2-anilino-5-nitro-anilino)-2-(tert-butoxycarbonylamino)-5-oxo-pentanoate (compound 41-1)

Boc-Glu-OMe (1.63 g, 0.00624 mol) was dissolved in dry DMF (15 mL). DIEA (2.97 mL, 0.0170 mol) was added followed by 4-nitro-N1-phenyl-benzene-1,2-diamine (1.30 g, 0.00567 mol; 13-4) and COMU (2.91 g, 0.00681 mol). The reaction was stirred at rt overnight. Most of the DMF was removed by evaporation under vacuum, then toluene (10 mL) was added, and the organic phase was washed with water (3×100 mL). The organic phase was dried over MgSO₄, filtered and the solvent was evaporated to give the title compound (41-1) as a darkbrown, oily, crude product (5.61 g). HPLC purity: 48%; (ESI+) m/z 373 [M+H-BOC]+. The material was used in next step without further purification.

### Methyl (2S)-2-amino-4-(5-nitro-1-phenyl-benzimidazol-2-yl)butanoate (compound 41-2)

To a solution of the crude methyl (2S)-5-(2-anilino-5-nitro-anilino)-2-(tert-butoxycarbonylamino)-5-oxo-pentanoate (5.61 g, 48% purity; 41-1) in EtOH (100 mL) was added 12 M HCl (15 mL) and the mixture was stirred at 70° C. for 45 minutes. The reaction mixture was diluted with water (100 mL), carefully neutralized to pH 7 with 4M NaOH (approx. 45 mL) and extracted with EtOAc (3×100 mL). The organic phase was dried over MgSO₄, filtered and the solvent was evaporated to give the title compound (41-2) as a dark reddish-brown solid product (2.04 g). HPLC purity: 60%; MS (ESI+) m/z 355 [M+H]⁺. The material was used in next step without further purification.

### Methyl (2S)-2-(tert-butoxycarbonylamino)-4-(5-nitro-1-phenyl-benzimidazol-2-yl)butanoate (compound 41-3)

To a solution of the crude methyl (2S)-2-amino-4-(5-nitro-1-phenyl-benzimidazol-2-yl)butanoate (2.04 g) in THF (25 mL) was added TEA (2.38 mL) and tert-butoxycarbonyl tertbutyl carbonate (1.49 g) and the mixture was stirred at room temperature for 1 h. The solvent was evaporated, and the residue was dissolved in EtOAc. The organic phase was washed with 5% citric acid, dried over MgSO₄, filtered and then the solvent was evaporated. The crude was purified by flash chromatography on silica eluting with 20 to 80% EtOAc in petroleum ether as gradient to give the title compound (41-3) (1.13 g, 0.00249 mmol, 44% yield). HPLC purity: 95%; MS (ESI+) m/z 455 [M+H]⁺.

### Methyl (2S)-4-(5-amino-1-phenyl-benzimidazol-2-yl)-2-(tert-butoxycarbonylamino)butanoate (compound 41-4)

To a solution of the methyl (2S)-2-(tert-butoxycarbonylamino)-4-(5-nitro-1-phenyl-benzimidazol-2-yl)butanoate (0.330 g, 0.000581 mol, 80% purity) in MeOH (20 mL) was added Pd/C (10.0%, 0.0734 g, 0.000069 mol) and it was evacuated. Hydrogen gas in balloon was attached and the mixture was stirred at room temperature for 4h. The Pd/C was removed filtration and the solvent was evaporated to dryness to afford methyl (2S)-4-(5-amino-1-phenyl-benzimidazol-2-yl)-2-(tertbutoxycarbonylamino) butanoate (190 mg, 0.000636 mol; 41-4). HPLC purity: 90%; MS (ESI+) m/z 425 [M+H]⁺.

### Methyl (2S)-4-[5-[bis(2-chloroethyl)amino]-1-phenyl-benzimidazol-2-yl]-2-(tert-butoxycarbonylamino)butanoate (compound 41-5)

NaBH₃CN (120 mg) was added to a mixture of methyl (2S)-4-(5-amino-1-phenyl-benzimidazol-2-yl)-2-(tert-butoxycarbonylamino)butanoate (90%, 300 mg, 0.636 mmol, 90% purity), 2-chloroacetaldehyde (50%, 0.24 mL) and TFA (0.19 mL) in EtOH (20 mL). The mixture was stirred at rt. for 3h. 3 extra equivalents of NaBH₃CN, 2-chloroacetaldehyde and TFA were added, and the reaction was stirred at rt for 1h. Toluene and sat. NaHCO₃ was added and the organic phase was separated and washed with water. The solvent was evaporated and the light brown solid residue was purified by flash chromatography on silica eluting with 50 to 100% EtOAc in petroleum·ether to afford methyl (2S)-4-[5-[bis(2-chloroethyl)amino]-1-phenyl-benzimidazol-2-yl]-2-(tert-butoxycarbonylamino)butanoate (190 mg, 0.000311 mol, 49% yield). HPLC purity: 90%; MS (ESI+) m/z=550 [M+H]⁺.

### (2S)-2-Amino-4-[5-[bis(2-chloroethyl)amino]-1-phenyl-benzimidazol-2-yl]butanoic acid; dihydrochloride (Example compound 41)

Methyl (2S)-4-[5-[bis(2-chloroethyl)amino]-1-phenyl-benzimidazol-2-yl]-2-(tert-butoxycarbonylamino)butanoate (190 mg, 0.000311 mol) was dissolved in 1,4-dioxane (5.00 mL), and then water (1.5 mL) and LiOH·H₂O (44 mg) were added. The mixture was stirred at 50° C. for 20 min after which water (10 mL) and DCM (10 mL) was added and the pH was adjusted to 6-7 with 1M HCl (aq.). The phases were separated, and the organic phase was washed with water and dried over MgSO₄. HCl (4M in 1,4-dioxane, 0.50 mL, 0.004 mol) was added to the organic phase and the clear solution stirred for 1h at rt. The solvent phase was removed from the precipitate, which was washed with diethyl ether. It was then redissolved in a small amount of water and immediately freeze dried to afford (2S)-2-amino-4-[5-[bis(2-chloroethyl)amino]-1-phenyl-benzimidazol-2-yl]butanoic acid; dihydrochloride (Example compound 41) (138 mg, 0.000242 mol, 78% yield) as a solid. HPLC purity: 89%; MS (ESI+) m/z=435 [M+H]⁺.

### Example 42: (2S)-2-[[(2S)-2-amino-3-[5-[bis(2-chloroethyl)amino]-1-methyl-benzimidazol-2-yl]propanoyl]amino]-3-methyl-butanoic acid; 2,2,2-trifluoroacetic acid (Example compound 42)

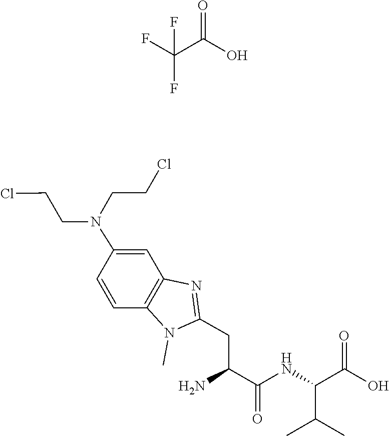 [SMILES: O=C(O)C(F)(F)F ; CC(C)[C@H](NC(=O)[C@@H](N)Cc1nc2cc(N(CCCl)CCCl)ccc2n1C)C(=O)O]

Ethyl (2S)-2-[[(2S)-2-amino-3-[5-[bis(2-chloroethyl)amino]-1-methyl-benzimidazol-2-yl]propanoyl]amino]-3-methyl-butanoate; dihydrochloride (compound 6) (12 mg, 0.02 mmol) was dissolved in a mixture of THF (1 mL), MeOH (0.5 mL), and 0.5M LiOH (aq, 0.13 mL, 0.065 mmol). The reaction mixture was stirred at room temperature over night. The organic solvents were evaporated, and the residue purified by preparative HPLC to afford (2S)-2-[[(2S)-2-amino-3-[5-[bis(2-chloroethyl)amino]-1-methyl-benzimidazol-2-yl]propanoyl]amino]-3-methyl-butanoic acid; 2,2,2-trifluoroacetic acid (Example compound 42) (2.6 mg, 0.00454 mmol, 21% yield) as a solid. HPLC purity: 95%; MS (ESI+) m/z=458 [M+H]⁺.

### Example 43: (2S)-2-[[(2S)-2-amino-4-methyl-pentanoyl]amino]-3-[5-[bis(2-chloroethyl)amino]-1-methyl-benzimidazol-2-yl]propanoic acid; dihydrochloride (Example compound 43)

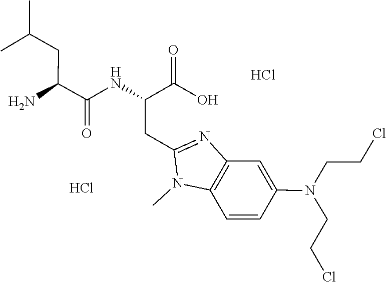 [SMILES: CC(C)C[C@H](N)C(=O)N[C@@H](Cc1nc2cc(N(CCCl)CCCl)ccc2n1C)C(=O)O ; Cl ; Cl]

To ethyl (2S)-2-[[(2S)-2-amino-4-methyl-pentanoyl]amino]-3-[5-[bis(2-chloroethyl)amino]-1-methyl-benzimidazol-2-yl]propanoate; dihydrochloride (Example compound 10) (12 mg, 0.02 mmol) was added 1M HCl (aq) (1 mL) and the reaction was stirred at room temperature overnight. Conc. HCl was added (10 drops). After four hours, an additional 0.3 mL of conc. HCl was added, and the mixture stirred overnight. The mixture was transferred to a round bottomed flask, water was added to the mixture and it was lyophilized to afford (2S)-2-[[(2S)-2-amino-4-methyl-pentanoyl]amino]-3-[5-[bis(2-chloroethyl)amino]-1-ethyl-benzimidazol-2-yl]propanoic acid; dihydrochloride (Example compound 43) (5 mg, 0.00917 mmol, 44% yield) as a solid. HPLC purity: 95%; MS (ESI+) m/z=472 [M+H]⁺.

¹H NMR (600 MHz, DMSO-d6) δ 14.78 (s, 1H), 9.40 (d, J=7.9 Hz, 1H), 8.38 (s, 3H), 7.76 (d, J=9.2 Hz, 1H), 7.16 (dd, J=9.2, 2.4 Hz, 1H), 6.94 (d, J=2.3 Hz, 1H), 4.93 (td, J=7.9, 5.7 Hz, 1H), 3.96 (s, 3H), 3.80 (dt, J=33.0, 6.8 Hz, 9H), 3.64 (dd, J=15.5, 5.9 Hz, 1H), 3.60-3.56 (m, 2H), 1.67-1.60 (m, 1H), 1.50 (t, J=7.2 Hz, 2H), 0.85 (t, J=7.2 Hz, 6H).

### Example 44: (2S)-2-[[(2S)-2-amino-3-[5-[bis(2-chloroethyl)amino]-1-methyl-benzimidazol-2-yl]propanoyl]amino]-4-methyl-pentanoic acid; dihydrochloride (Example compound 44)

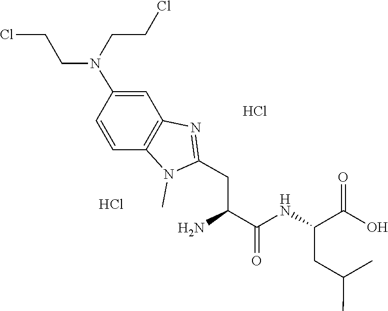 [SMILES: CC(C)C[C@H](NC(=O)[C@@H](N)Cc1nc2cc(N(CCCl)CCCl)ccc2n1C)C(=O)O ; Cl ; Cl]

To ethyl (2S)-2-[[(2S)-2-amino-3-[5-[bis(2-chloroethyl)amino]-1-methyl-benzimidazol-2-yl]propanoyl]amino]-4-methyl-pentanoate; dihydrochloride (Example compound 4) (10 mg, 0.017 mmol) was added 1M HCl (aq) (1 mL) and the reaction was stirred at room temperature overnight. HCl conc. was added (10 drops) and the reaction was stirred overnight. The mixture was transferred to a round bottomed flask, diluted with water and lyophilized to afford (2S)-2-[[(2S)-2-amino-3-[5-[bis(2-chloroethyl)amino]-1-methyl-benzimidazol-2-yl]propanoyl]amino]-4-methyl-pentanoic acid; dihydrochloride (Example compound 44) (5 mg, 0.00917 mmol, 53% yield) as a solid. HPLC purity: 95%; MS (ESI+) m/z=472 [M+H]⁺.

¹H NMR (600 MHz, DMSO-d6) δ 14.61 (s, 1H), 9.11 (d, J=7.7 Hz, 1H), 8.73 (s, 3H), 7.75 (d, J=9.3 Hz, 1H), 7.16 (d, J=9.4 Hz, 1H), 6.92 (d, J=2.4 Hz, 1H), 4.51-4.44 (m, 1H), 4.27 (q, J=7.6 Hz, 1H), 3.94 (s, 3H), 3.87-3.75 (m, 9H), 3.66 (d, J=6.6 Hz, 2H), 1.76-1.68 (m, 1H), 1.55 (dt, J=8.8, 4.6 Hz, 2H), 0.88 (dd, J=19.5, 6.5 Hz, 6H).

### Example 45: Ethyl (2R)-2-[[(2S)-2-amino-4-[5-[bis(2-chloroethyl)amino]-1-methyl-benzimidazol-2-yl]butanoyl]amino]-4-methyl-pentanoate di-2,2,2-trifluoroacetic acid (Example compound 45)

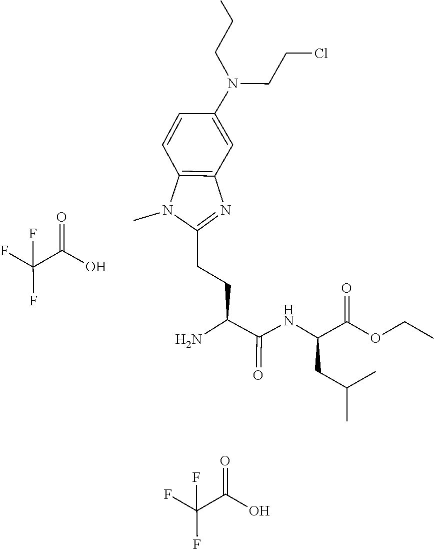 [SMILES: O=C(O)C(F)(F)F ; CCCN(CCCl)c1ccc2c(c1)nc(CC[C@H](N)C(=O)N[C@H](CC(C)C)C(=O)OCC)n2C ; O=C(O)C(F)(F)F]

Example 45 was synthesized according to Scheme 12 below:

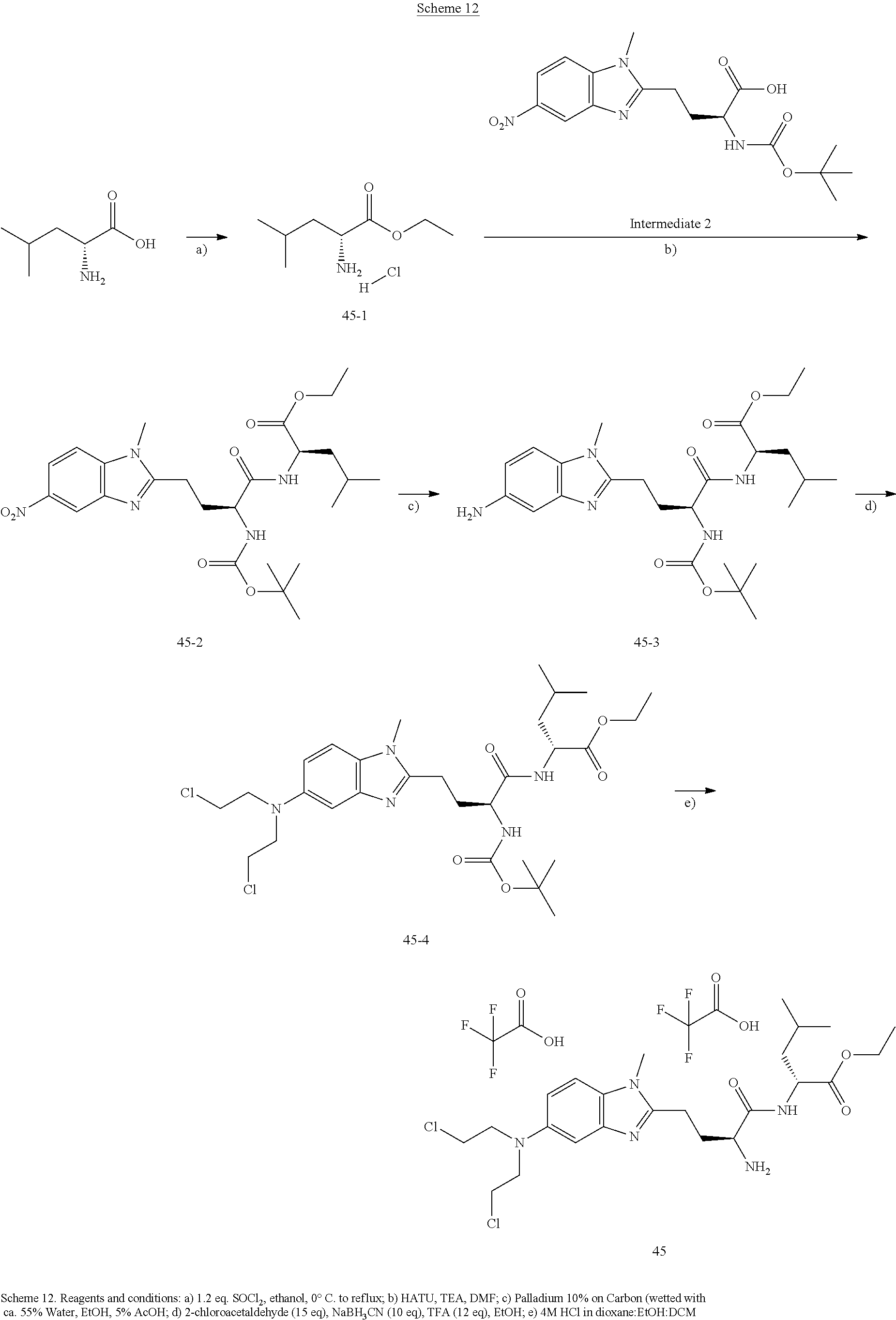

### R-Leucine ethyl ester hydrochloride (compound 45-1)

In a 25 mL flask, (2R)-2-amino-4-methyl-pentanoic acid (1.00 g, 0.00762 mol) and anhydrous ethanol (6.00 mL) were added. The reaction was chilled on ice-bath and thionyl chloride (0.667 mL, 0.00915 mol) was added dropwise giving a slurry. After addition the reaction was stirred at reflux for 2h 30 min. The solvent was evaporated, and the residue was suspended in Et₂O (16 mL) and stirred for 1h. The product was filtered of to give the title compound (45-1) (1.36 g, 0.00693 mol, 91% yield). HPLC purity: 99%; MS (ESI+) m/z=160 [M+H]⁺

### Ethyl ((S)-2-((tert-butoxycarbonyl)amino)-4-(1-methyl-5-nitro-1H-benzo[d]imidazol-2-yl)butanoyl)-D-leucinate (compound 45-2)

To a mixture of (2S)-2-(tert-butoxycarbonylamino)-4-(1-methyl-5-nitro-benzimidazol-2-yl)butanoic acid (Intermediate 2) (0.500 g, 0.00132 mol), R-leucine ethyl ester hydrochloride (0.259 g, 0.00132 mol) and TEA (0.553 mL, 0.00396 mol) in DMF (4.60 mL) was added HATU (0.502 g, 0.00132 mol). The reaction was stirred for 2h and then water (9 mL) was added. The product was oiling out. The liquid was removed by pipette. To the wet semi-solid was added EtOH (5 mL). The semi-solid was dissolved by heating to 80° C. Water (5 mL) was added until sign of precipitation. The heating was turned off. The product was oiling out. The solvent was evaporated, and the residue was dissolved in toluene and washed by water. The organic phase was dried over MgSO₄, filtered and the solvent was evaporated to give the title compound (45-2) (0.575 g, 0.00111 mol, yield: 84%). HPLC purity: 97%; MS (ESI+) m/z=520 [M+H]⁺. The material was used without further purification in next step.

### Ethyl (2R)-2-[[(2S)-4-(5-amino-1-methyl-benzimidazol-2-yl)-2-(tert-butoxycarbonylamino)butanoyl]amino]-4-methyl-pentanoate (compound 45-3)

A mixture of ethyl (2R)-2-[[(2S)-2-(tert-butoxycarbonylamino)-4-(1-methyl-5-nitro-benzimidazol-2-yl)butanoyl]amino]-4-methyl-pentanoate (575 mg, 0.00111 mol) and palladium 10% on carbon (wetted with ca. 55% Water (0.262 g, 0.00111 mol) were stirred in EtOH (25.0 mL) containing AcOH (1.30 mL) overnight. Solid materials were filtered of on celite to give the title compound (542 mg, 1.11 mmol, 100% yield). HPLC purity: 98%%; MS (ESI+) m/z=490 [M+H]⁺. The material was used without further purification in next step.

### Ethyl (2R)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate (compound 45-4)

At room temperature NaBH₃CN (0.348 g, 0.00554 mol) was added to a mixture of ethyl (2R)-2-[[(2S)-4-(5-amino-1-methyl-benzimidazol-2-yl)-2-(tert-butoxycarbonylamino)butanoyl]amino]-4-methyl-pentanoate (0.542 g, 0.00111 mol; 45-3), 2-chloroacetaldehyde (50%, 0.701 mL, 0.00554 mol) and TFA (0.493 mL, 0.00664 mol) in EtOH (30 mL). After 20 minutes of stirring additional NaBH₃CN (0.348 g, 0.00554 mol) and 2-chloroacetaldehyde (5.0%, 0.701 mL, 0.00554 mol) were added. The reaction was further stirred for 20 minutes before 2-chloroacetaldehyde (50%, 0.701 mL, 0.00554 mol) was added. The reaction was further stirred for 2 hours whereafter toluene was added and the organic phase was washed by sodium bicarbonate and brine. The organics were dried over MgSO₄, filtered and the solvent was evaporated. The compound was purified by flash chromatography on spheric silica gel eluting with 2% EtOH in DCM to give the title compound 45-4 (209 mg, 0.340 mmol, 31% yield). HPLC purity: 95%; MS (ESI+) m/z=614 [M+H]⁺.

### Ethyl (2R)-2-[[(2S)-2-amino-4-[5-[bis(2-chloroethyl)amino]-1-methyl-benzimidazol-2-yl]butanoyl]amino]-4-methyl-pentanoate di-2,2,2-trifluoroacetic acid (compound Example 45)

4M HCl in dioxane (1.40 mL) was added to ethyl (2R)-2-[[(2S)-4-[5-[bis(2-chloroethyl)amino]-1-methyl-benzimidazol-2-yl]-2-(tert-butoxycarbonylamino)butanoyl]amino]-4-methyl-pentanoate (0.209 g, 0.000340 mol) in a mixture of DCM (1.40 mL) and EtOH (0.860 mL). The reaction was stirred for 5h and then the solvent was evaporated, giving the crude product which was 90% pure. The crude product was dissolved in MeCN containing 30% water and purified on preparative HPLC, ACE column eluting with 10 to 60% MeCN/water (0.1% TFA) to give the title compound (Example compound 45) (104 mg, 0,140 mmol, 49% yield). HPLC purity: 99%; MS (ESI+) m/z=514 [M+H]⁺.

¹H NMR (600 MHz, DMSO-d6 δ 9.18-8.99 (m, 1H), 8.41 (s, 3H), 7.70-7.59 (m, 1H), 7.13-7.02 (m, 1H), 6.94 (d, J=2.3 Hz, 1H), 4.33 (q, J=7.8 Hz, 1H), 4.13 (q, J=7.1 Hz, 2H), 4.07-4.01 (m, 1H), 3.85-3.71 (m, 11H), 3.17-3.07 (m, 2H), 2.25 (q, J=7.4 Hz, 2H), 1.65-1.52 (m, 3H), 1.21 (t, J=7.1 Hz, 3H), 0.89 (d, J=6.1 Hz, 3H), 0.86 (d, J=6.1 Hz, 3H).

### Example 46: Ethyl (2S)-2-[[(2R)-2-amino-4-[5-[bis(2-chloroethyl)amino]-1-methyl-benzimidazol-2-yl]butanoyl]amino]-4-methyl-pentanoate (Example compound 46)

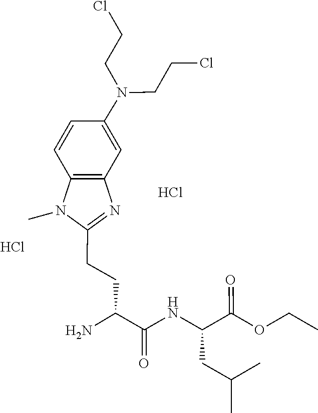 [SMILES: Cl ; CCOC(=O)[C@H](CC(C)C)NC(=O)[C@H](N)CCc1nc2cc(N(CCCl)CCCl)ccc2n1C ; Cl]

Example 46 was synthesized according to Scheme 13 below:

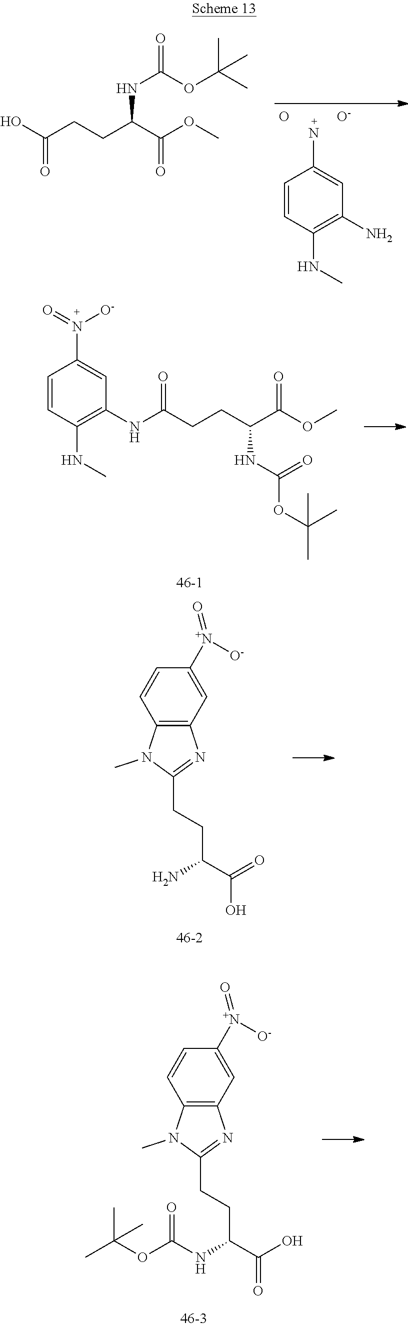

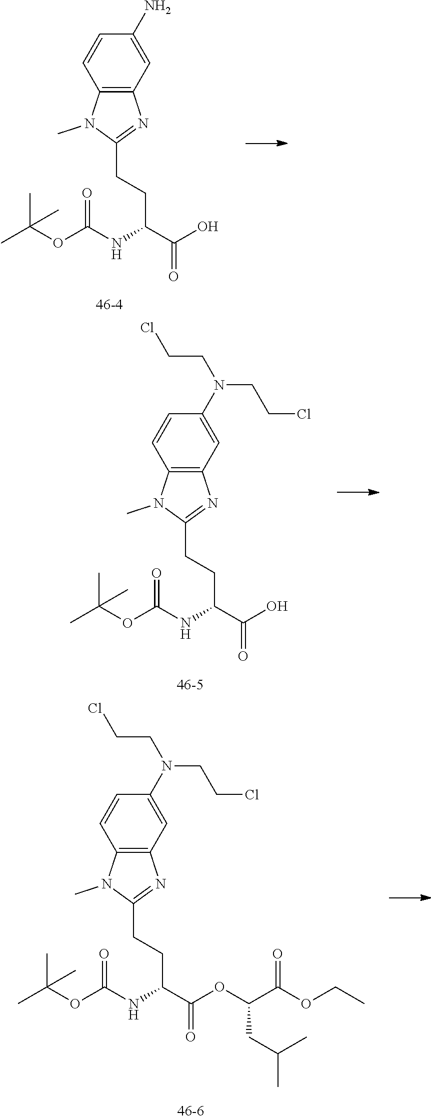 [SMILES: Cn1c(CC[C@@H](NC(=O)OC(C)(C)C)C(=O)O)nc2cc(N)ccc21 ; Cn1c(CC[C@@H](NC(=O)OC(C)(C)C)C(=O)O)nc2cc(N(CCCl)CCCl)ccc21 ; CCOC(=O)[C@H](CC(C)C)OC(=O)[C@@H](CCc1nc2cc(N(CCCl)CCCl)ccc2n1C)NC(=O)OC(C)(C)C]

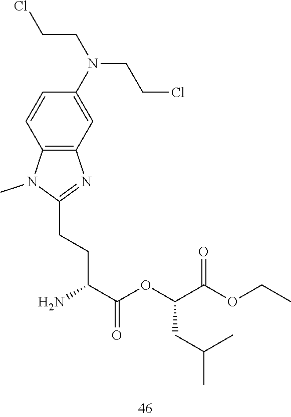 [SMILES: CCOC(=O)[C@H](CC(C)C)OC(=O)[C@H](N)CCc1nc2cc(N(CCCl)CCCl)ccc2n1C]

### Methyl (2R)-2-(tert-butoxycarbonylamino)-5-[2-(methylamino)-5-nitro-anilino]-5-oxo-pentanoate (compound 46-1)

To a solution of N-tert-butoxycarbonyl-D-glutamic acid α-methyl ester (1.00 eq, 2.00 g, 7.65 mmol), 1-methylimidazole (1.00 eq, 0.61 mL, 7.65 mmol) and N1-methyl-4-nitro-benzene-1,2-diamine (1.34 g, 8.037 mmol) in MeCN (20 mL) was slowly added chloro(dimethylamino)-N,N-dimethylmethanaminium hexafluorophosphate (TCFH) (1.10 eq, 2.36 g, 8.42 mmol). The mixture was stirred at room temperature for 1h and then water (200 mL) was added. The organic phase was passed through a phase separator cartridge, evaporated, and dried under vacuum to afford methyl (2R)-2-(tert-butoxycarbonylamino)-5-[2-(methylamino)-5-nitro-anilino]-5-oxo-pentanoate (3.1 g, 7.55 mmol, 99% yield) as a solid. HPLC purity: 99%; MS (ESI+) m/z=843 [2M+Na]+

### (2R)-2-Amino-4-(1-methyl-5-nitro-benzimidazol-2-yl)butanoic acid (compound 46-2)

Methyl (2R)-2-(tert-butoxycarbonylamino)-5-[2-(methylamino)-5-nitro-anilino]-5-oxo-pentanoate (1.00 eq, 3.10 g, 7.55 mmol) was added to hydrochloric acid (4M, aq.) (18.0 eq, 34 mL, 136 mmol). After addition, the mixture was heated and stirred at 100° C. without stopper for 2h. The reaction was allowed to reach room temperature and a small amount of brown solid was filtered off. The filtrate was cooled on an ice bath and 10M NaOH was carefully added until pH2 was reached. After that, 4M NaOH solution was added until pH 5.2 was obtained. The solid obtained was filtered, washed with water and Et₂O and dried under vacuum to afford (2R)-2-amino-4-(1-methyl-5-nitro-benzimidazol-2-yl)butanoic acid (2.3 g, 7.52 mmol, >99% yield) as a solid. HPLC purity: 91%; MS (ESI+) m/z=279 [M+H]⁺

### (2R)-2-(tert-butoxycarbonylamino)-4-(1-methyl-5-nitro-benzimidazol-2-yl)butanoic acid (compound 46-3)

To a solution of (2R)-2-amino-4-(1-methyl-5-nitro-benzimidazol-2-yl)butanoic acid (1.00 eq, 2.30 g, 7.52 mmol) in water (10 mL) was added NaOH (4M, aq) (1.10 eq, 2.1 mL, 8.27 mmol). To the solution was added di-tert-butyl dicarbonate (1.40 eq, 2.4 mL, 10.5 mmol) and the mixture was stirred at room temperature for 2h. A precipitate was formed. Additional 4M NaOH (0.5 eq.) was added, and the reaction was stirred at room temperature for 3h. The pH was adjusted to 9.2 and the reaction mixture was washed with Et₂O (3×300 mL). The organic phase obtained was discarded and the aqueous phase was evaporated to remove the Et₂O residue. 4M HCl was added dropwise to the aqueous phase. The solid obtained was collected, washed with water, and dried to afford (2R)-2-(tert-butoxycarbonylamino)-4-(1-methyl-5-nitro-benzimidazol-2-yl)butanoic acid (1.85 g, 4.59 mmol, 61% yield) as a solid. HPLC purity: 94%; MS (ESI+) m/z=379 [M+H]⁺.

### (2R)-4-(5-Amino-1-methyl-benzimidazol-2-yl)-2-(tert-butoxycarbonylamino)butanoic acid (compound 46-4)

(2R)-2-(tert-Butoxycarbonylamino)-4-(1-methyl-5-nitro-benzimidazol-2-yl)butanoic acid (1.00 eq, 300 mg, 0.769 mmol) was dissolved in ethanol (5 mL) and placed in a H-reactor. Pd/C 10% (0.0500 eq, 8.2 mg, 0.0385 mmol) was added and the reactor was sealed. A nitrogen purge was conducted. H₂ was charged to the reactor at 3 bar and the reaction was stirred at room temperature for 2h. Hydrogen was vented with a nitrogen purge. Catalyst was removed by filtration through Celite. The solvent was evaporated to afford (2R)-4-(5-amino-1-methyl-benzimidazol-2-yl)-2-(tert-butoxycarbonylamino)butanoic acid (235 mg, 0.690 mmol, 94% yield). HPLC purity: 95%; MS (ESI+) m/z=349 [M+H]⁺

### (2R)-4-[5-[bis(2-Chloroethyl)amino]-1-methyl-benzimidazol-2-yl]-2-(tert-butoxycarbonylamino)butanoic acid (compound 46-5)

(2R)-4-(5-Amino-1-methyl-benzimidazol-2-yl)-2-(tert-butoxycarbonylamino)butanoic acid (1.00 eq, 253 mg, 0.690 mmol) was suspended in ethanol (2 mL). Sodium cyanoborohydride (3.00 eq, 130 mg, 2.07 mmol) was added followed by trifluoroacetic acid (1.50 eq, 0.079 mL, 1.03 mmol) and then chloroacetaldehyde (50%, 3.00 eq, 0.26 mL, 2.07 mmol) was added dropwise. The reaction mixture was stirred for 30 minutes. 2 extra equivalents of NaBH₃CN, chloroacetaldehyde and TFA were added, and the reaction was stirred at room for 2h. The pH was adjusted 3.6 by adding 4M NaOH dropwise. The ethanol was evaporated, water was added, and the aqueous phase was extracted with EtOAc. The organic phase was evaporated and the crude (2R)-4-[5-[bis(2-chloroethyl)amino]-1-methyl-benzimidazol-2-yl]-2-(tert-butoxycarbonylamino)butanoic acid (303 mg, 0.557 mmol, 81% yield) was isolated as a solid. HPLC purity: 87%; MS (ESI+) m/z=473 [M+H]⁺

### Ethyl (2S)-2-[[(2R)-4-[5-[bis(2-chloroethyl)amino]-1-methyl-benzimidazol-2-yl]-2-(tert-butoxycarbonylamino)butanoyl]amino]-4-methyl-pentanoate (compound 46-6)

1-(3-Dimethylaminopropyl)-3-ethylcarbodiimide hydrochloride (1.15 eq, 41 mg, 0.211 mmol) was added to a mixture of (2R)-4-[5-[bis(2-chloroethyl)amino]-1-methyl-benzimidazol-2-yl]-2-(tert-butoxycarbonylamino)butanoic acid (1.00 eq, 100 mg, 0.184 mmol), ethyl (2S)-2-amino-4-methyl-pentanoate (1.15 eq, 34 mg, 0.211 mmol), ethyl cyanoglyoxylate-2-oxime (1.00 eq, 26 mg, 0.184 mmol) and 4-methylmorpholine (1.15 eq, 23 uL, 0.211 mmol) in MeCN (3 mL). The reaction mixture was stirred at room temperature overnight. The reaction mixture was filtrated and purified by preparative chromatography. The pure fractions were freeze dried to afford ethyl (2S)-2-[[(2R)-4-[5-[bis(2-chloroethyl)amino]-1-methyl-benzimidazol-2-yl]-2-(tert-butoxycarbonylamino)butanoyl]amino]-4-methyl-pentanoate (17 mg, 0.0275 mmol, 15% yield). HPLC purity: 99%; MS (ESI+) m/z=614 [M+H]⁺.

### Ethyl (2S)-2-[[(2R)-2-amino-4-[5-[bis(2-chloroethyl)amino]-1-methyl-benzimidazol-2-yl]butanoyl]amino]-4-methyl-pentanoate; dihydrochloride (Example compound 46)

Ethyl (2S)-2-[[(2R)-4-[5-[bis(2-chloroethyl)amino]-1-methyl-benzimidazol-2-yl]-2-(tert-butoxycarbonylamino)butanoyl]amino]-4-methyl-pentanoate (1.00 eq, 17.1 mg, 0.0275 mmol) was dissolved in MeCN (2 mL) and hydrochloric acid (4M in dioxane) (5.00 eq, 35 uL, 0.139 mmol) was added. The reaction was stirred at room temperature for 1h. The mixture was filtered and purified by preparative chromatography and the pure fractions were pooled and freeze dried to afford ethyl (2S)-2-[[(2R)-2-amino-4-[5-[bis(2-chloroethyl)amino]-1-methyl-benzimidazol-2-yl]butanoyl]amino]-4-methyl-pentanoate; dihydrochloride (Example compound 46) (14.3 mg, 0.0234 mmol, 84% yield) as a solid. HPLC purity: 96%; MS (ESI+) m/z=514 [M+H]⁺.

¹H NMR (600 MHz, DMSO-d6) δ 14.81 (s, 1H), 9.23 (d, J=7.6 Hz, 1H), 8.59 (s, 3H), 7.75 (d, J=9.1 Hz, 1H), 7.15 (d, J=9.2 Hz, 1H), 6.94 (d, J=2.3 Hz, 1H), 4.30-4.24 (m, 1H), 4.11 (q, J=7.1 Hz, 2H), 4.04 (s, 1H), 3.89 (s, 3H), 3.83 (t, J=6.7 Hz, 4H), 3.77 (t, J=6.6 Hz, 4H), 3.2-3-3 (m, 2H), 2.32 (dp, J=29.8, 7.6 Hz, 2H), 1.69-1.57 (m, 2H), 1.53 (ddd, J=13.8, 8.5, 5.6 Hz, 1H), 1.20 (t, J=7.1 Hz, 3H), 0.87 (dd, J=21.4, 6.3 Hz, 6H).

### Example 47: Ethyl (2R)-2-[[(2R)-2-amino-4-[5-[bis(2-chloroethyl)amino]-1-methyl-benzimidazol-2-yl]butanoyl]amino]-4-methyl-pentanoate; dihydrochloride (Example compound 47)

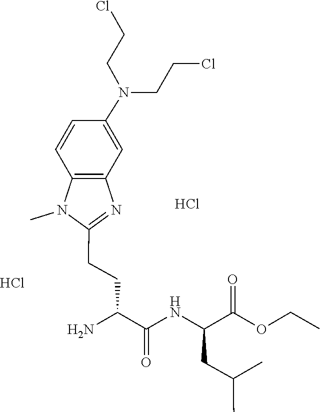 [SMILES: Cl ; CCOC(=O)[C@@H](CC(C)C)NC(=O)[C@H](N)CCc1nc2cc(N(CCCl)CCCl)ccc2n1C ; Cl]

Example 47 was synthesized according to Scheme 13 but using D-Leucine, ethyl ester, hydrochloride (1.15 eq, 41 mg, 0.211 mmol) as starting material in the peptide coupling with compound 46-5 to give ethyl (2R)-2-[[(2R)-4-[5-[bis(2-chloroethyl) amino]-1-methyl-benzimidazol-2-yl]-2-(tert-butoxycarbonylamino)butanoyl]amino]-4-methyl-pentanoate; 2,2,2-trifluoroacetic acid (27 mg, 0.0375 mmol, 20% yield), isolated as a solid. HPLC purity: 99%; MS (ESI+) m/z=614 [M+H]⁺.

The ethyl (2R)-2-[[(2R)-2-amino-4-[5-[bis(2-chloroethyl)amino]-1-methyl-benzimidazol-2-yl]butanoyl]amino]-4-methyl-pentanoate; dihydrochloride (Example compound 47) (6 mg, 0.0097 mmol, 25% yield) was isolated as a solid. HPLC purity: 95%; MS (ESI+) m/z=514 [M+H]⁺.

¹H NMR (600 MHz, DMSO-d6) δ 14.79 (s, 1H), 9.25 (d, J=7.0 Hz, 1H), 8.69-8.47 (m, 3H), 7.75 (d, J=9.1 Hz, 1H), 7.15 (d, J=9.2 Hz, 1H), 6.93 (s, 1H), 4.31 (dt, J=11.3, 5.7 Hz, 1H), 4.16-4.04 (m, 3H), 3.90 (s, 3H), 3.83 (d, J=6.7 Hz, 4H), 3.78 (d, J=6.4 Hz, 4H), 3.38-3.33 (m, 2H), 2.32 (dp, J=23.6, 7.6, 7.0 Hz, 2H), 1.75 (h, J=6.9 Hz, 1H), 1.64 (td, J=12.3, 10.8, 5.1 Hz, 1H), 1.55 (ddd, J=14.0, 9.4, 4.8 Hz, 1H), 1.18 (t, J=7.2 Hz, 3H), 0.90 (dd, J=25.1, 6.5 Hz, 6H).

### Example 48: Ethyl (2S)-2-[[(2S)-2-amino-4-[6-[bis(2-chloroethyl)amino]-3-methyl-imidazo[4,5-b] pyridin-2-yl]butanoyl]amino]-4-methyl-pentanoate di-2,2,2-trifluoroacetic acid (Example compound 48)

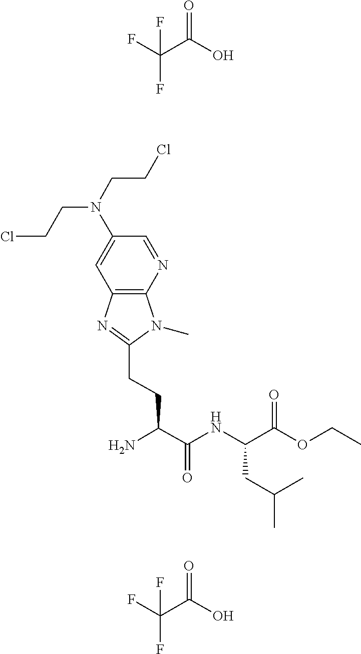 [SMILES: O=C(O)C(F)(F)F ; CCOC(=O)[C@H](CC(C)C)NC(=O)[C@@H](N)CCc1nc2cc(N(CCCl)CCCl)cnc2n1C ; O=C(O)C(F)(F)F]

Example 48 was synthesized according to Scheme 14 below:

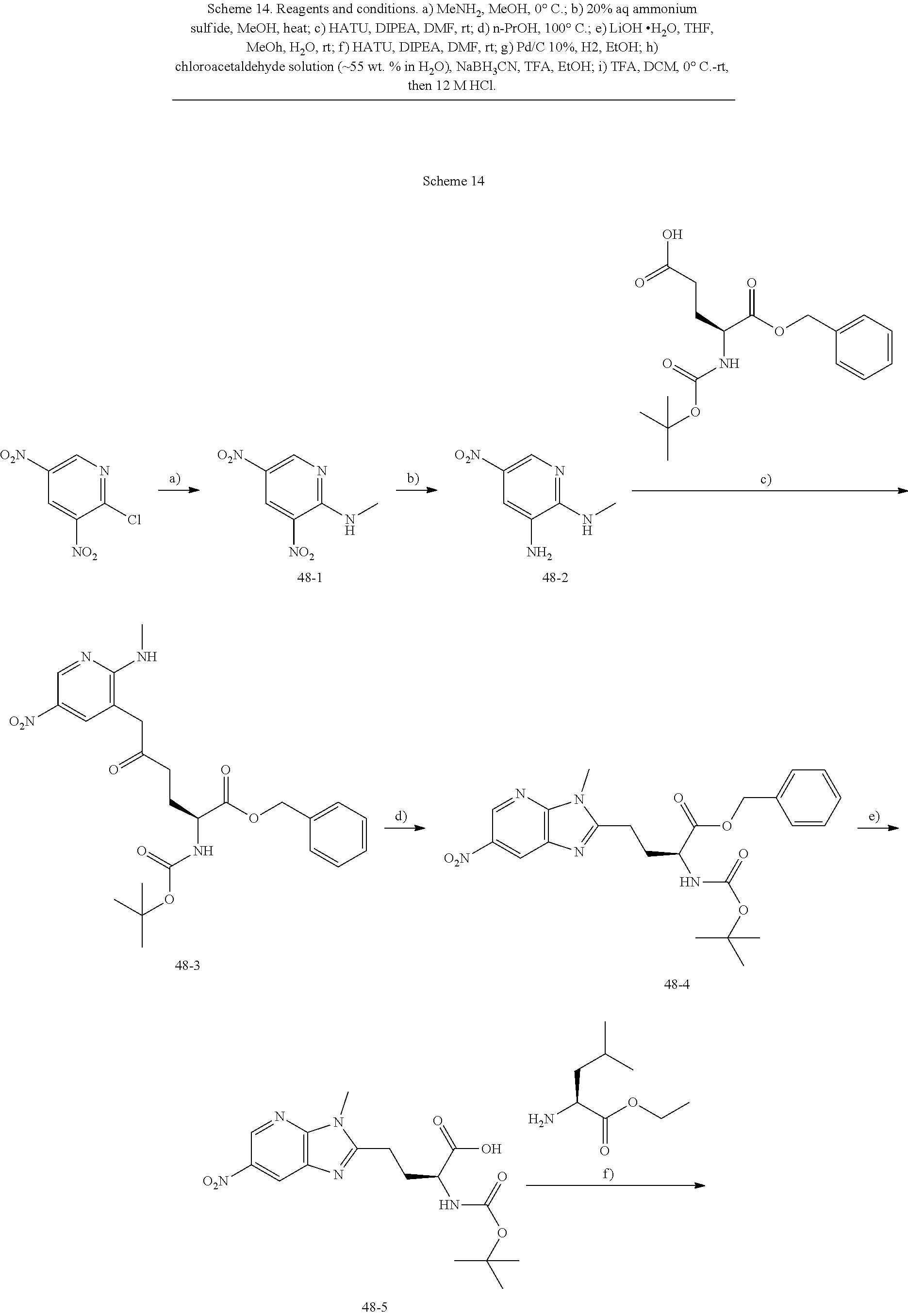

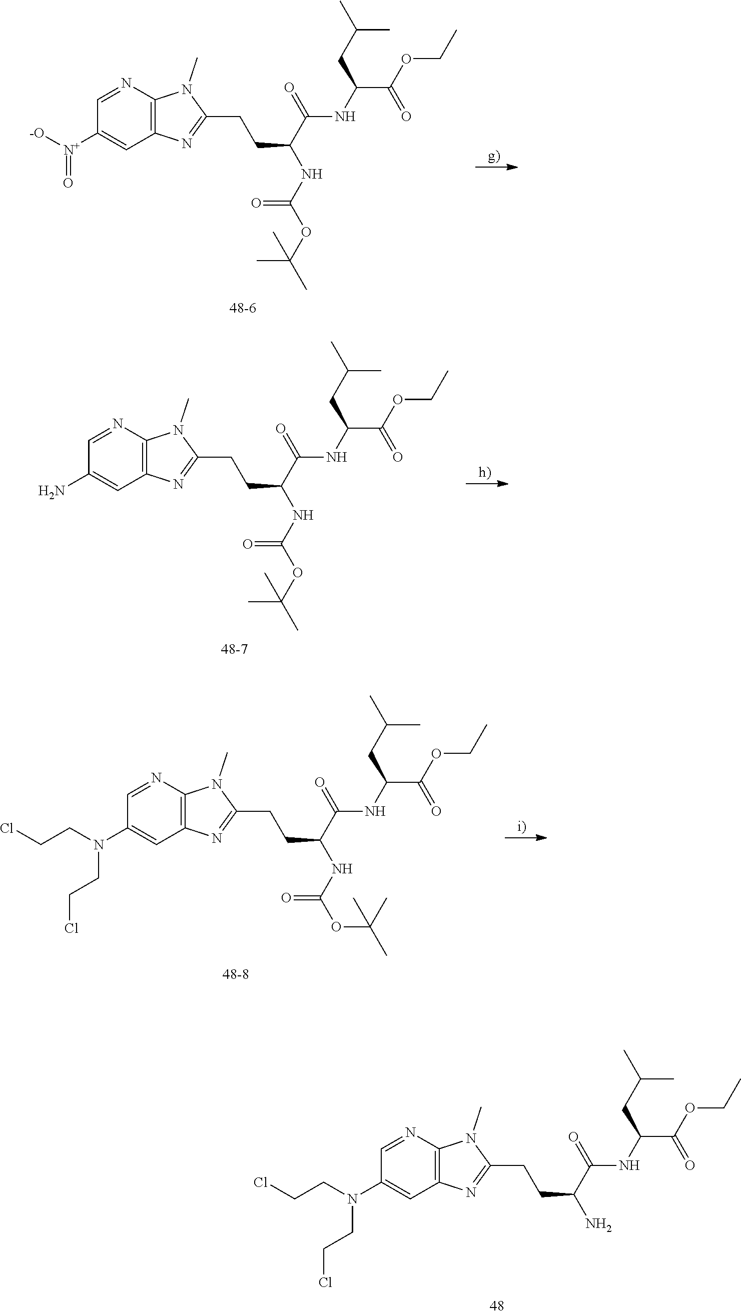

### N-Methyl-3,5-dinitro-pyridin-2-amine (compound 48-1)

To a suspension of 2-chloro-3,5-dinitropyridine (4.0 g, 0.0196 mol) in MeOH (40 mL) was added dropwise, at 0-5° C., a mixture of an aqueous solution of MeNH₂ (4.32 mL, 40 vol %, 0.059 mol) and MeOH (16 mL). A yellow precipitate was formed, and was collected by filtration, washed with MeOH and water, and dried under vacuum overnight to give N-methyl-3,5-dinitro-pyridin-2-amine (3.00 g, 0.0151 mol, 77% yield). HPLC purity: 95%; MS (ESI+) m/z=199 [M+H]⁺.

### N2-Methyl-5-nitro-pyridine-2,3-diamine (compound 48-2)

N-Methyl-3,5-dinitro-pyridin-2-amine (2.80 g, 14.1 mmol) was suspended in methanol (60 mL), and a 20% aqueous solution of ammonium sulfide (24.1 mL) was added. The temperature was increased to 75° C. and the mixture stirred for 2.5 h. The reaction was then stirred at rt overnight. The mixture was placed in an ice bath and a precipitate was formed. The solid was collected by filtration and dried overnight under vacuum to give N2-Methyl-5-nitro-pyridine-2,3-diamine (3.39 g, more than theoretical yield) as red-brownish solid. The material was taken to the next step without further purification. HPLC purity: 90%; MS (ESI+) m/z=169 [M+H]⁺.

### Benzyl (2S)-2-(tert-butoxycarbonylamino)-5-[[2-(methylamino)-5-nitro-3-pyridyl]amino]-5-oxo-pentanoate (compound 48-3)

To a solution of (4S)-5-benzyloxy-4-(tert-butoxycarbonylamino)-5-oxo-pentanoic acid (1.00 g, 2.97 mmol) in dimethylformamide (7.50 mL) were added HATU (1.36 g, 3.57 mmol) and DIEA (1.04 mL, 5.45 mmol) and the reaction was stirred for 2 min before N2-methyl-5-nitro-pyridine-2,3-diamine (compound 48-2) (0.500 g, 2.97 mmol) was added. The reaction was stirred at rt for 22 h. EtOAc was added, and the organic phase was washed with brine (3×), dried over magnesium sulfate, decanted and concentrated to give benzyl (2S)-2-(tert-butoxycarbonylamino)-5-[[2-(methylamino)-5-nitro-3-pyridyl] amino]-5-oxo-pentanoate (2.74 g, more than theoretical yield). The material was taken to the next step without further purification. HPLC purity: 80%; MS (ESI+) m/z=488 [M+H]⁺.

### Benzyl (2S)-2-(tert-butoxycarbonylamino)-4-(3-methyl-6-nitro-imidazo[4,5-b]pyridin-2-yl)butanoate (compound 48-4)

Benzyl (2S)-2-(tert-butoxycarbonylamino)-5-[[2-(methylamino)-5-nitro-3-pyridyl]amino]-5-oxo-pentanoate (1.07 g, 2.19 mmol) in 1-propanol (20.0 mL) was heated at 100° C. for 6 days (slower reaction as compared to when performed on a smaller scale). The 1-propanol was evaporated and EtOAc was added. The organic phase was washed with brine, dried over magnesium sulfate and concentrated. The crude material was purified by flash chromatography (SiO₂, 12 g, petroleum ether/EtOAc 0-100%) to give benzyl (2S)-2-(tert-butoxycarbonylamino)-4-(3-methyl-6-nitro-imidazo[4,5-b] pyridin-2-yl)butanoate (280 mg, 27% yield). HPLC purity: 90%; MS (ESI+) m/z=470 [M+H]⁺.

### (2S)-2-(tert-Butoxycarbonylamino)-4-(3-methyl-6-nitro-imidazo[4,5-b]pyridin-2-yl) butanoic acid (compound 48-5)

To a solution of benzyl (2S)-2-(tert-butoxycarbonylamino)-4-(3-methyl-6-nitro-imidazo[4,5-b] pyridin-2-yl)butanoate (0.280 g, 0.596 mmol) in tetrahydrofuran (1.20 mL)/methanol (1.20 mL) containing water (0.2 mL) was added LiOH—H₂O (0.0425 g, 1.01 mmol). The reaction was stirred at rt for 2 h. EtOAc and 5% citric acid were added, and the phases separated. The organic phase was dried over magnesium sulfate, filtered and the solvent was evaporated to give (2S)-2-(tert-butoxycarbonylamino)-4-(3-methyl-6-nitro-imidazo[4,5-b]pyridin-2-yl) butanoic acid (0.240 g). The material was taken to the next step without further purification. HPLC purity: 95%; MS (ESI+) m/z=380 [M+H]⁺.

### Ethyl (2S)-2-[[(2S)-2-(tert-butoxycarbonylamino)-4-(3-methyl-6-nitro-imidazo[4,5-b]pyridin-2-yl)butanoyl]amino]-4-methyl-pentanoate (compound 48-6)

To a solution of (2S)-2-(tert-butoxycarbonylamino)-4-(3-methyl-6-nitro-imidazo[4,5-b]pyridin-2-yl)butanoic acid (0.240 g, 0.633 mmol) in N,N-dimethylformamide (1.50 mL) were added ethyl (2S)-2-amino-4-methyl-pentanoate; hydrochloride (0.124 g, 0.633 mmol) and HATU (0.289 g, 0.759 mmol) followed by N,N-diisopropylethylamine (0.204 g, 1.58 mmol). The reaction was then stirred at rt overnight. EtOAc was added and the organic phase was washed with brine (3×), dried over MgSO₄, filtered and concentrated. The crude material was purified by flash chromatography (SiO₂, petroleum ether/EtOAc 0-100%) to give ethyl (2S)-2-[[(2S)-2-(tert-butoxycarbonylamino)-4-(3-methyl-6-nitro-imidazo[4,5-b]pyridin-2-yl)butanoyl]amino]-4-methyl-pentanoate (0.340 g). HPLC purity: 90%; MS (ESI+) m/z=521 [M+H]⁺.

### Ethyl (2S)-2-[[(2S)-4-(6-amino-3-methyl-imidazo[4,5-b]pyridin-2-yl)-2-(tert-butoxy carbonylamino)butanoyl]amino]-4-methyl-pentanoate (compound 48-7)

A mixture of ethyl (2S)-2-[[(2S)-2-(tert-butoxycarbonylamino)-4-(3-methyl-6-nitro-imidazo[4,5-b]pyridin-2-yl)butanoyl]amino]-4-methyl-pentanoate (0.340 g, 0.653 mmol) and Pd/C 10% (0.0695 g, 0.653 mmol) in ethanol (20.0 mL) was stirred under H₂ (atmospheric pressure) overnight. The catalyst was filtered off and the solvent evaporated to give ethyl (2S)-2-[[(2S)-4-(6-amino-3-methyl-imidazo[4,5-b]pyridin-2-yl)-2-(tert-butoxy carbonylamino)butanoyl]amino]-4-methyl-pentanoate (0.273 g, 85% yield) of the title compound as a transparent, sticky oil. The material was taken to the next step without further purification. HPLC purity: 90%; MS (ESI+) m/z=491 [M+H]⁺.

### Ethyl (2S)-2-[[(2S)-4-[6-[bis(2-chloroethyl)amino]-3-methyl-imidazo[4,5-b]pyridin-2-yl]-2-(tert-butoxycarbonylamino)butanoyl]amino]-4-methyl-pentanoate (compound 48-8)

To ethyl (2S)-2-[[(2S)-4-(6-amino-3-methyl-imidazo[4,5-b]pyridin-2-yl)-2-(tert-butoxy carbonyl amino)butanoyl]amino]-4-methyl-pentanoate (170 mg, 0.346 mmol), chloroacetaldehyde (0.22 mL, 50% solution in water) and TFA (0.12 mL) were added at rt. Sodium cyanoborohydride (109 mg, 1.73 mmol) was then added and the reaction was stirred at rt for 30 min, at which time point 50% conversion was observed. Again, the same amounts of all the above reagents were added and the reaction stirred for another 30 min. The volatiles were evaporated under vacuum. The crude product was dissolved in ethyl acetate and washed with aqueous saturated sodium carbonate solution, brine, dried over sodium sulfate, filtered, and concentrated. The crude material was purified by flash chromatography (12 g silica column, 70-90% MeOH in DCM) to afford ethyl (2S)-2-[[(2S)-4-[6-[bis(2-chloroethyl)amino]-3-methyl-imidazo[4,5-b]pyridin-2-yl]-2-(tert-butoxycarbonylamino)butanoyl]amino]-4-methyl-pentanoate (75 mg, 35% yield) of the title compound as a thick colourless oil. HPLC purity: 95%; MS (ESI+) m/z=615 [M+H]⁺.

### Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate; di-2,2,2-trifluoroacetic acid (Example compound 48)

To a solution of ethyl (2S)-2-[[(2S)-4-[6-[bis(2-chloroethyl)amino]-3-methyl-imidazo[4,5-b]pyridin-2-yl]-2-(tert-butoxycarbonylamino)butanoyl]amino]-4-methyl-pentanoate (compound 48-8) (35 mg, 0.057 mmol) in DCM (2 mL) was added TFA (0.1 mL) at 5° C. The reaction was stirred at 5° C. for 30 min before it was allowed to warm to rt. Additional TFA (0.2 mL) was then added and the reaction stirred at rt for 3 h. The crude material was purified by reverse phase acidic preparative HPLC (10-90% acetonitrile, 0.1% TFA in water). The pure fractions were concentrated to afford ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate; di-2,2,2-trifluoroacetic acid (Example compound 48) (11.3 mg, 39% yield) as a solid. HPLC purity: 95%; MS (ESI+) m/z=515 [M+H]⁺.

¹H NMR (400 MHz, DMSO-d6) δ 9.04 (d, J=7.2 Hz, 1H), 8.57-8.26 (m, 3H), 8.10 (d, J=2.6 Hz, 1H), 7.43 (d, J=2.7 Hz, 1H), 4.38-4.30 (m, 1H), 4.12-4.08 (m, 2H), 4.05-4.02 (m, 1H), 3.81-3.73 (m, 11H), 3.13 (t, J=8.0 Hz, 2H), 2.39-2.22 (m, 2H), 1.77-1.67 (m, 1H), 1.65-1.52 (m, 2H), 1.17 (t, J=7.1 Hz, 3H), 0.93 (d, J=6.6 Hz, 3H), 0.89 (d, J=6.5 Hz, 3H).

### Example 49: (2S)-2-amino-4-[5-[bis(2-chloro-1,1,2,2-tetradeutero-ethyl)amino]-1-methyl-benzimidazol-2-yl]butanoic acid; dihydrochloride (Example compound 49)

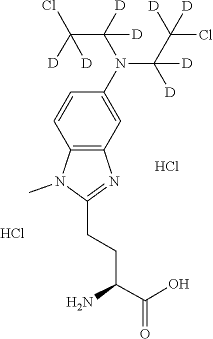 [SMILES: Cl ; [2H]C([2H])(Cl)C([2H])([2H])N(c1ccc2c(c1)nc(CC[C@H](N)C(=O)O)n2C)C([2H])([2H])C([2H])([2H])Cl ; Cl]

Example 49 was synthesized according to Scheme 15 below:

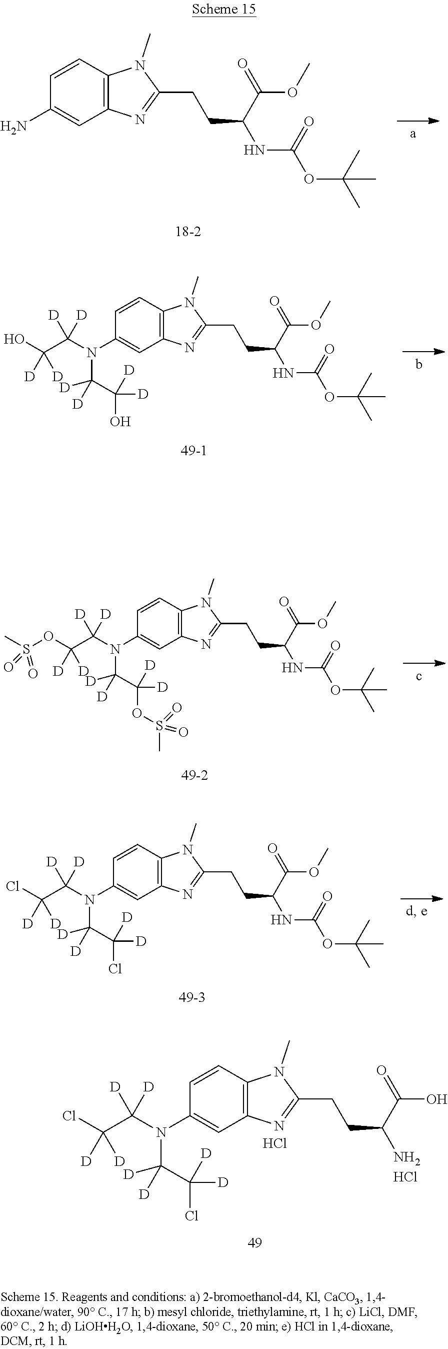

### Methyl (2S)-4-[5-[bis(1,1,2,2-tetradeutero-2-hydroxy-ethyl)amino]-1-methyl-benz imidazol-2-yl]-2-(tert-butoxycarbonylamino)butanoate (compound 49-1)

To a solution of methyl (2S)-4-(5-amino-1-methyl-benzimidazol-2-yl)-2-(tert-butoxycarbonylamino)butanoate (compound 18-2) (50 mg, 0.138 mmol) in a 1,4-dioxane (1.00 mL)/water (0.500 mL) mixture in a closed reaction tube were added CaCO₃ (21.5 mg, 0.214 mmol) and a catalytic amount of KI (5.1 mg, 0.0306 mmol) followed by 2-bromoethanol-d4 (39.5 mg, 0.306 mmol). The reaction was heated at 90° C. for 17 hours after which the mixture was diluted with acetonitrile and purified by preparative HPLC on an XBridge column eluting with 10 to 80% MeCN in water containing 50 mM NH₄HCO₃. The title compound (49-1) was obtained as a white solid, containing up to 19% monoalkylated and 2% overalkylated by-products (29 mg). MS (ESI+) m/z=459 [M+H]⁺.

### Methyl (2S)-4-[5-[bis(1,1,2,2-tetradeutero-2-methylsulfonyloxy-ethyl)amino]-1-methyl-benzimidazol-2-yl]-2-(tert-butoxycarbonylamino)butanoate (compound 49-2)

Methyl (2S)-4-[5-[bis(1,1,2,2-tetradeutero-2-hydroxy-ethyl)amino]-1-methyl-benz imidazol-2-yl]-2-(tert-butoxycarbonylamino)butanoate (29 mg, 0.06 mmol) was dissolved in dry dichloromethane (1.00 mL) and cooled to 0° C. Triethylamine (20 mg, 0.20 mmol) and mesyl chloride (0.0096 mL, 0.124 mmol) were then added and the mixture stirred at room temperature until complete conversion. Water was added and the DCM phase washed and dried to give the title compound (49-2) as a brown oily crude product which was used in the next step without further purification (31 mg). MS (ESI+) m/z=615 [M+H]⁺.

### Methyl (2S)-4-[5-[bis(2-chloro-1,1,2,2-tetradeutero-ethyl)amino]-1-methyl-benz imidazol-2-yl]-2-(tert-butoxycarbonylamino)butanoate (compound 49-3)

Methyl (2S)-4-[5-[bis(1,1,2,2-tetradeutero-2-methylsulfonyloxy-ethyl)amino]-1-methyl-benzimidazol-2-yl]-2-(tert-butoxycarbonylamino)butanoate (21 mg, 0.035 mmol) was dissolved in dry dimethylformamide (0.50 mL) then LiCl (30 mg, 0.70 mmol) was added and the mixture stirred at 60° C. until complete conversion. The mixture was purified by flash chromatography on silica using 50 to 100% EtOAc in petroleum ether to give an off-white oily product (14 mg, 92% yield). HPLC purity: 90%; MS (ESI+) m/z=495 [M+H]⁺.

### (2S)-2-Amino-4-[5-[bis(2-chloro-1,1,2,2-tetradeutero-ethyl)amino]-1-methyl-benz imidazol-2-yl]butanoic acid dihydrochloride (Example compound 49)

Methyl (2S)-4-[5-[bis(2-chloro-1,1,2,2-tetradeutero-ethyl)amino]-1-methyl-benz imidazol-2-yl]-2-(tert-butoxycarbonylamino)butanoate (13 mg, 0.026 mmol) was dissolved in 1,4-dioxane (1.0 L) then water (0.3 mL) and LiOH·H₂O (26 mg, 0.063 mmol) was added. The mixture was stirred at 50° C. for 20 minutes after which water (2 mL) and dichloromethane (1 mL) was added and the pH was set to 5-6 with aqueous HCl solution. The phases were separated, and the organic phase washed with water and dried. To the dried organic phase excess HCl in 1,4-dioxane (4 mol/L) was added and the clear solution stirred for 30 minutes at room temperature. The mixture was concentrated to dryness to afford (2S)-2-amino-4-[5-[bis(2-chloro-1,1,2,2-tetradeutero-ethyl)amino]-1-methyl-benz imidazol-2-yl]butanoic acid dihydrochloride (Example compound 49) (6 mg, 55% yield) as a solid. HPLC purity: 97%; MS (ESI+) m/z=381 [M+H]⁺.

### Example 50: Ethyl (2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoate; dihydrochloride (Example compound 50)

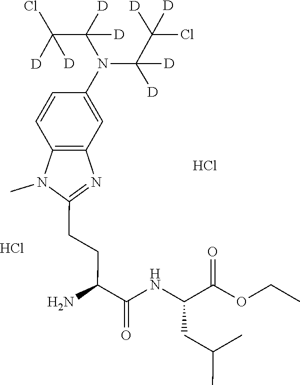 [SMILES: Cl ; [2H]C([2H])(Cl)C([2H])([2H])N(c1ccc2c(c1)nc(CC[C@H](N)C(=O)N[C@@H](CC(C)C)C(=O)OCC)n2C)C([2H])([2H])C([2H])([2H])Cl ; Cl]

Example 50 was synthesized according to Scheme 16 below:

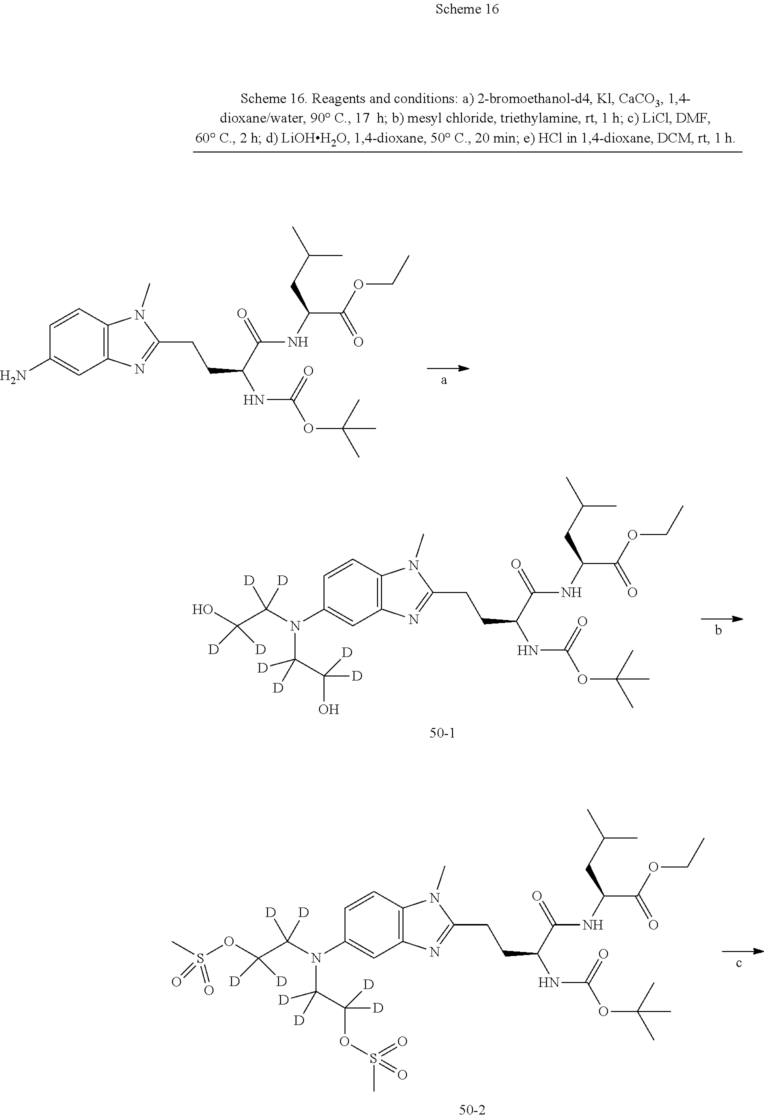

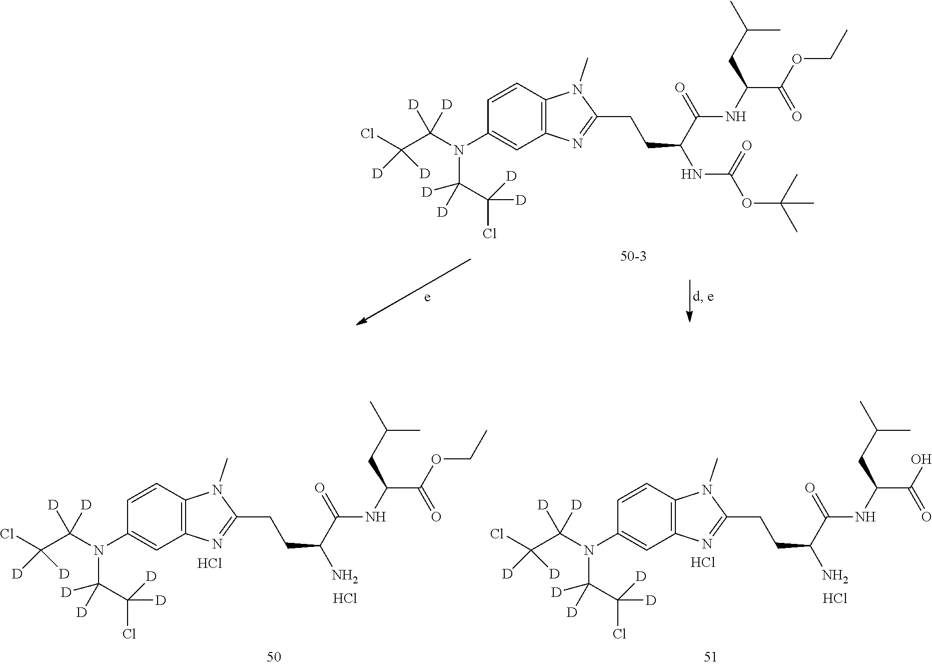

### Ethyl (2S)-2-[[(2S)-4-[5-[bis(1,1,2,2-tetradeutero-2-hydroxy-ethyl)amino]-methyl-benzimidazol-2-yl]-2-(tert-butoxycarbonylamino)butanoyl]amino]-1-methyl-pentanoate (compound 50-1)

The title compound (50-1) was prepared from ethyl (2S)-2-[[(2S)-4-(5-amino-1-methyl-benzimidazol-2-yl)-2-(tert-butoxycarbonylamino)butanoyl]amino]-4-methyl-pentanoate (prepared as described in step 2, towards the synthesis of Example 10 compound 1), according to the procedure described for synthesis of compound 49-1 (39 mg, 65% yield). HPLC purity: 80%; MS (ESI+) m/z=586 [M+H]⁺.

### Ethyl (2S)-2-[[(2S)-4-[5-[bis(1,1,2,2-tetradeutero-2-hydroxy-ethyl)amino]-1-methyl-benzimidazol-2-yl]-2-(tertbutoxycarbonylamino)butanoyl]amino]-4-methyl-pentanoate (compound 50-2)

The title compound (50-2) was prepared from compound 50-1 according to the procedure described for synthesis of compound 49-2 (26 mg, 71% yield). MS (ESI+) m/z=742 [M+H]⁺.

### Ethyl (2S)-2-[[(2S)-4-[5-[bis(2-chloro-1,1,2,2-tetradeutero-ethyl)amino]-1-methyl-benzimidazol-2-yl]-2-(tert-butoxycarbonylamino)butanoyl]amino]-4-methyl-pentanoate (compound 5-3)

The title compound (50-3) was prepared from compound 50-2 according to the procedure described for synthesis of compound 49-3 (20 mg, 92% yield). HPLC purity: 90%; MS (ESI+) m/z=622 [M+H]⁺.

### Ethyl (2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoate (Example 10 compound 50)

The title compound (Example compound 50) was prepared from compound 50-3 according to the procedure described for step 5 in the synthesis of Example compound 1, with slightly modified conditions (DCM as solvent, 1h as reaction time) (9 mg, 4% yield). HPLC purity: 95%; MS (ESI+) m/z=522 [M+H]⁺.

### Example 51: (2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoic acid; dihydrochloride (Example compound 51)

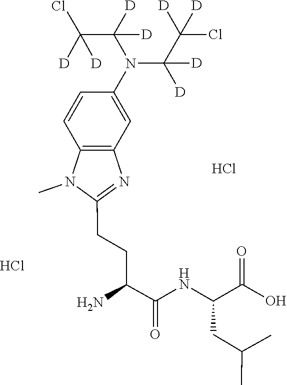 [SMILES: Cl ; [2H]C([2H])(Cl)C([2H])([2H])N(c1ccc2c(c1)nc(CC[C@H](N)C(=O)N[C@@H](CC(C)C)C(=O)O)n2C)C([2H])([2H])C([2H])([2H])Cl ; Cl]

Example 51 was synthesized according to Scheme 16. The compound was prepared from compound 50-3 according to the procedure described as last steps in the synthesis of Example compound 49 (8 mg, 68% yield). HPLC purity: 95%; MS (ESI+) m/z=494 [M+H]⁺.

### Biological Examples

### Biological Example 1—Assessment of In Vitro Cytotoxic Potency in a MM.1S Cell Viability Assay

Methods: MM.1S Cells were cultivated in RPMI medium 1640 (IX) supplemented with 10 μg/μL penicillin/streptomycin, 10% heat inactivated FBS, 0.1 mM Sodium Pyruvate, 1×MEM NEAA (ThermoFisher) and 1 mM HEPES. Cells were grown to reach log phase before being exposed to the test compounds.

Test compounds (Example compounds 1 to 36, 38 to 41 and 45 to 48) and comparative compounds (bendamustine, busulfan, melphalan, chlorambucil and 4-hydroperoxy cyclophosphamide) were dissolved in 100% DMSO and added to a 384 assay plate. Additional DMSO was added to maintain the same final DMSO concentration (0.2%) in all wells. 25 μL cell suspension with a concentration of 100000 cells/mL was added to each well. The cells were cultured in a CO₂ incubator at 37° C. for 72 h. Plates were equilibrated to room temperature for 30 minutes before adding 20 μL CellTiter-Glo® 2.0 (Promega) reagent to all wells. The plate was then allowed to stabilize for 20 minutes before recording the luminescence (emission filter 700 nm) in an Envision plate reader. The plate reader data was normalized to negative controls (no treatment) vs positive controls (cells treated with 400 μM Chlorpromazine for 72 h) and IC₅₀ was derived from a 4 Parameter Logistic regression curve fitting.

Results: Test compounds were evaluated on their ability to inhibit MM.1S cell proliferation and cause cytotoxicity in vitro. Dose responses could be extracted from experiments and are shown in Table 1 below. The test compounds displayed good cytotoxicity towards MM.1S cells. Example compounds 1 to 16, 21 to 32, 34 to 36, 38 and 45 to 48 were found to be more cytotoxic than bendamustine, busulfan, melphalan, chlorambucil and 4-hydroperoxy cyclophosphamide in this assay.

TABLE 1
Cytotoxicity in MM.1S cells

| | | MM.1S, | |
|---|---|---|---|
| | MM.1S | IC50, | |
| | IC50 | st dev | |
| Example No | (μM) | (μM) | MM.1S, n |
| 1 | 0.0068 | 0.0022 | 20 |
| 2 | 0.0078 | 0.0029 | 7 |
| 3 | 0.0068 | 0.0012 | 2 |
| 4 | 0.0066 | 0.0017 | 2 |
| 5 | 0.0226 | 0.0079 | 2 |
| 6 | 0.0987 | 0.0381 | 4 |
| 7 | 0.0229 | 0.0108 | 2 |
| 8 | 0.0144 | 0.0031 | 2 |
| 9 | 0.0225 | 0.0025 | 2 |
| 10 | 0.0192 | 0.0037 | 8 |
| 11 | 0.0147 | 0.0011 | 2 |
| 12 | 0.0125 | 0.0045 | 2 |
| 13 | 0.049 | 0.0249 | 7 |
| 14 | 0.0087 | 0.0012 | 2 |
| 15 | 0.0091 | 0.0007 | 2 |
| 16 | 0.025 | 0.0076 | 2 |
| 17 | 39.967 | 4.5325 | 2 |
| 18 | 20.244 | 20.968 | 14 |
| 19 | 4.0199 | 0.6051 | 2 |
| 20 | 5.753 | 1.149 | 6 |
| 21 | 0.0266 | 0.0016 | 3 |
| 22 | 0.0313 | 0.0018 | 2 |
| 23 | 0.0418 | 0.0017 | 2 |
| 24 | 0.1561 | 0.0247 | 2 |
| 25 | 0.0185 | 0.002 | 3 |
| 26 | 0.0068 | 0.0019 | 2 |
| 27 | 0.0082 | 0.0014 | 2 |
| 28 | 0.352 | — | 1 |
| 29 | 0.0014 | — | 1 |
| 30 | 0.0397 | 0.0081 | 4 |
| 31 | 0.181 | 0.008 | 2 |
| 32 | 0.0143 | 0.002 | 3 |
| 33 | 79.872 | 13.613 | 2 |
| 34 | 0.0683 | 0.0087 | 8 |
| 35 | 0.0867 | 0.0087 | 2 |
| 36 | 0.0575 | 0.0047 | 2 |
| 38 | 0.052 | 0.0158 | 7 |
| 39 | >1.996 | — | 1 |
| 40 | 7.177 | 0.709 | 2 |
| 41 | 16.507 | 4.3134 | 2 |
| 45 | 0.512 | 0.1113 | 3 |
| 46 | 0.0591 | 0.0137 | 3 |
| 47 | 1.7124 | 0.7595 | 3 |
| 48 | 0.205 | 0.0262 | 2 |
| Bendamustine | 23.963 | 6.7392 | 12 |
| Melphalan | 4.2451 | 0.7017 | 10 |
| Busulfan | 180.3 | — | 1 |
| Chlorambucil | 6.873 | 6.4333 | 2 |
| 4-hydroperoxy | 7.2953 | — | 1 |
| cyclophosphamide | | | |

### Biological Example 2—Assessment of In Vitro Cytotoxicity in Haematological Cell Lines, Human Peripheral Blood Mononuclear Cells and Normal Fibroblasts

Methods: Cell lines used for these experiments are listed in Table 2 below. All cells were cultivated in RPMI medium 1640 (IX). All media was supplemented with 10 μg/μl penicillin/streptomycin, 10% heat inactivated FBS, 0.1 mM Sodium Pyruvate, 1×MEM NEAA (ThermoFisher) and 1 mM HEPES. The DERL-2 cells had higher FBS (20%) and 40 ng/mL IL-2 was added. hPBMCs were thawed and allowed to acclimatise overnight and then stimulated with 10 μg/mL PHA-M and 40 ng/mL IL-2 for 4 hours before being exposed to test compounds. All cells, except for hPBMCs, were grown to reach log phase before being exposed to the test compounds.

TABLE 2
Cell lines used in in vitro cytotoxicity experiments

| Cell line ID | Description |
|---|---|
| U937 | Histiocytic Lymphoma |
| THP-1 | Acute Myeloid Leukemia |
| DOHH-2 | B Cell Lymphoma |
| HG-3 | Chronic Lymphocytic Leukemia (CLL) |
| SC-1 | B cell Lymphoma |
| SU-DHL-8 | B-cell non-Hodgkin Lymphoma |
| Toledo | Diffuse Large B Cell Lymphoma |
| DERL-2 | T cell Lymphoma |
| SUP-T1 | T cell Lymphoma |
| AMO-1 | Multiple Myeloma |
| EJM | Multiple Myeloma |
| KMS-12-BM | Multiple Myeloma |
| MM.1S | Multiple Myeloma |
| OPM-2 | Multiple Myeloma |
| RPMI8226 | Multiple Myeloma (resistant) |
| U266 | Multiple Myeloma |
| SK-MM-1 | Plasma Cell Leukemia |
| hPBMC | Human Peripheral blood mononuclear cells |
| BJ | Fibroblasts (normal cells) |

Test compounds were dissolved in 100% DMSO and added to a 384 assay plate. Additional DMSO was added to maintain the same final DMSO concentration (0.2%) in all wells. 25 μL cell suspension with a concentration of 100000 cells/mL was added to each well. The cells were cultured in a CO₂ incubator at 37° C. for 72 h. Plates were equilibrated to room temperature for 30 minutes before adding 20 μL CellTiter-Glo® 2.0 (Promega) reagent to all wells. The plate was then allowed to stabilize for 20 minutes before recording the luminescence (Em filter 700 nm) in an Envision plate reader. The plate reader data was normalized to negative controls (no treatment) vs positive controls (cells treated with 400 μM Chlorpromazine for 72 h) and IC₅₀ was derived from a 4 parameter Logistic regression curve fitting.

Results: Test compounds were evaluated on their ability to inhibit cell proliferation in vitro using several different cell lines of haematological origin. Cytotoxicity was also evaluated in non-malignant human peripheral blood mononuclear cells (hPBMC) and fibroblasts (BJ). Table 3 shows the dose responses extracted from the experiments for all compounds tested. In the table, the results are given as Cytotoxicity IC50 μM+/−standard deviation (number of tests). nd=values not determined.

Tables 3a and b. Cytotoxicity of Test Compounds in Normal and Cancerous Cell Lines

TABLE 3a

| Compound: | 1 | 2 | 10 | 13 |
|---|---|---|---|---|
| THP-1 | 0.016 +/− | nd | nd | nd |
| | 0 (1) | | | |
| U937 | 0.0098 +/− | 0.0142 +/− | 0.0194 +/− | nd |
| | 0.0025 (4) | 0.0042 (2) | 0.0049 (2) | |
| AMO-1 | 0.0497 +/− | nd | nd | 0.9646 +/− |
| | 0.0196 (3) | | | 0.2196 (3) |
| EJM | 0.0271 +/− | nd | nd | 0.356 +/− |
| | 0.0059 (2) | | | 0.0451 (2) |
| KMS-12-BM | 0.0079 +/− | nd | nd | 0.1373 +/− |
| | 0.002 (2) | | | 0.0142 (2) |
| MM.1S | 0.0068 +/− | 0.0078 +/− | 0.0192 +/− | 0.049 +/− |
| | 0.0022 (20) | 0.0029 (7) | 0.0037 (8) | 0.0249 (7) |
| OPM-2 | 0.1734 +/− | nd | nd | 2.1102 +/− |
| | 0.0576 (2) | | | 0.6334 (2) |
| RPMI-8226 | 0.0906 +/− | 0.0415 +/− | 0.3352 +/− | 1.7213 +/− |
| | 0.0549 (6) | 0.004 (2) | 0.0102 (2) | 0.391 (2) |
| SK-MM-1 | 0.0347 +/− | 0.0383 +/− | 0.0624 +/− | nd |
| | 0.024 (5) | 0.0316 (3) | 0.0288 (2) | |
| U266 | 0.0214 +/− | nd | nd | 0.4002 +/− |
| | 0.0085 (2) | | | 0.136 (2) |
| DOHH-2 | 0.0042 +/− | 0.0053 +/− | 0.0639 +/− | nd |
| | 0.0005 (4) | 0.0007 (2) | 0.0237 (2) | |
| HG-3 | 0.0257 +/− | 0.0257 +/− | 0.0713 +/− | nd |
| | 0.0052 (4) | 0.0103 (2) | 0.0069 (2) | |
| SC-1 | 0.0133 +/− | 0.021 +/− | 0.1686 +/− | 0.1344 +/− |
| | 0.0051 (5) | 0.0044 (2) | 0.0874 (2) | 0 (1) |
| SU-DHL-8 | 0.0199 +/− | 0.0204 +/− | 0.0221 +/− | nd |
| | 0.0032 (4) | 0.0009 (2) | 0.0054 (2) | |
| Toledo | 0.0045 +/− | 0.0066 +/− | 0.0085 +/− | nd |
| | 0.0026 (4) | 0.0021 (2) | 0.0003 (2) | |
| DERL-2 | 0.006 +/− | 0.0078 +/− | 0.0226 +/− | nd |
| | 0.0007 (3) | 0.0011 (3) | 0.0058 (3) | |
| SUP-T1 | 0.0127 +/− | 0.0178 +/− | 0.0356 +/− | nd |
| | 0.004 (3) | 0.0053 (3) | 0.0076 (3) | |
| BJ | 1.0745 +/− | nd | nd | >1.996 +/− |
| | 0 (1) | | | (1) |
| hPBMC | 0.0054 +/− | 0.0075 +/− | 0.0264 +/− | nd |
| | 0.0021 (2) | 0.002 (2) | 0.0097 (2) | |

TABLE 3b

| | | | | Benda- | |
|---|---|---|---|---|---|
| Compound: | 30 | 34 | 38 | mustine | Melphalan |
| THP-1 | nd | nd | nd | 40.204 +/− | 4.3058 +/− |
| | | | | 28.995 (2) | 0 (1) |
| U937 | nd | 0.3966 +/− | 0.0253 +/− | 27.061 +/− | nd |
| | | 0.0382 (2) | 0.0008 (2) | 7.409 (3) | |
| AMO-1 | 0.6528 +/− | 0.7576 +/− | nd | 56.977 +/− | 8.3424 +/− |
| | 0.0666 (3) | 0.1497 (3) | | 8.7068 (4) | 1.8996 (3) |
| EJM | 0.1734 +/− | 0.6368 +/− | nd | 64.64 +/− | 40.017 +/− |
| | 0.0191 (2) | 0.0192 (2) | | 2.4078 (3) | 1.2365 (2) |
| KMS-12-BM | 0.045 +/− | 0.1046 +/− | nd | 27.469 +/− | 9.829 +/− |
| | 0.0164 (2) | 0.0047 (2) | | 6.1077 (3) | 1.1877 (2) |
| MM.1S | 0.0397 +/− | 0.0683 +/− | 0.052 +/− | 23.963 +/− | 4.2451 +/− |
| | 0.0081 (4) | 0.0087 (8) | 0.0158 (7) | 6.7392 (12) | 0.7017 (10) |
| OPM-2 | 0.3218 +/− | 0.9609 +/− | nd | 81.878 +/− | 10.176 +/− |
| | 0.0557 (2) | 0.336 (2) | | 7.837 (3) | 1.7559 (2) |
| RPMI-8226 | 0.4924 +/− | 1.4476 +/− | 0.6764 +/− | 111.12 +/− | 32.486 +/− |
| | 0.0701 (2) | 0.2263 (2) | 0.1445 (2) | 34.705 (10) | 3.8841 (2) |
| SK-MM-1 | nd | 0.6039 +/− | 0.1752 +/− | 73.498 +/− | nd |
| | | 0.4592 (3) | 0.0525 (2) | 21.704 (3) | |
| U266 | 0.1314 +/− | 0.2661 +/− | nd | 83 +/− | 18.85 +/− |
| | 0.0024 (2) | 0.1386 (2) | | 41.993 (3) | 3.4229 (2) |
| DOHH-2 | nd | 0.5428 +/− | 0.0281 +/− | 10.701 +/− | nd |
| | | 0.1497 (2) | 0.0099 (2) | 5.9963 (2) | |
| HG-3 | nd | 0.8877 +/− | 0.0498 +/− | 25.787 +/− | nd |
| | | 0.1628 (2) | 0.0236 (2) | 13.589 (2) | |
| SC-1 | 0.0537 +/− | 1.0624 +/− | 0.0772 +/− | 25.141 +/− | 8.6422 +/− |
| | 0 (1) | 0.2814 (3) | 0.0116 (2) | 7.3961 (3) | 0 (1) |
| SU-DHL-8 | nd | 0.2022 +/− | 0.0714 +/− | 14.264 +/− | nd |
| | | 0.1099 (2) | 0.0244 (2) | 3.6275 (3) | |
| Toledo | nd | 0.3276 +/− | 0.0162 +/− | 12.519 +/− | nd |
| | | 0.0368 (2) | 0.0047 (2) | 6.2427 (3) | |
| DERL-2 | nd | nd | nd | 21.071 +/− | 1.8822 +/− |
| | | | | 3.1768 (3) | 0.3506 (3) |
| SUP-T1 | nd | nd | nd | 33.527 +/− | 2.9645 +/− |
| | | | | 11.372 (3) | 0.371 (3) |
| BJ | >1.996 +/− | >1.996 +/− | nd | >598.8 +/− | 103.64 +/− |
| | (1) | (1) | | (2) | 38.599 (2) |
| hPBMC | nd | nd | nd | 15.803 +/− | 3.6018 +/− |
| | | | | 6.6175 (2) | 1.8923 (2) |

It is seen that the compounds of the invention are consistently more cytotoxic than the two compounds of the prior art.

### Biological Example 3—Assessment of Cellular Retention/Efflux Example 18 (after Treatment with Example 1), Bendamustine and Melphalan

Methods: MMA1S cells at 2×10⁶ cells/mL were seeded at 250 μL per well in a 96-well plate. Cells were incubated in the presence of a test compound for 5, 15, and 60 min before pelleting cells by centrifugation at +4° C. for 5 min. Cell media was immediately harvested to a second plate and stored at −80° C. until further analysis. Pelleted cells were washed once with ice cold PBS before storage at −80° C. until further analysis.

Cells were lysed and protein in the media were precipitated using acetonitrile dimethylformamide, 9:1 containing internal standard. Samples were centrifuged and the supernatant was analyzed with LC-MS/MS (ACQUITY UPLC-Xevo TQ-S micro) to determine compound concentration.

Example compound 1, bendamustine and melphalan were used as test compounds.

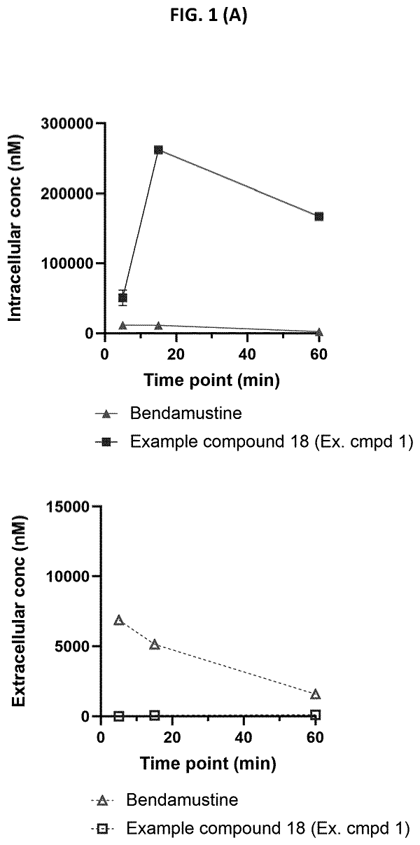

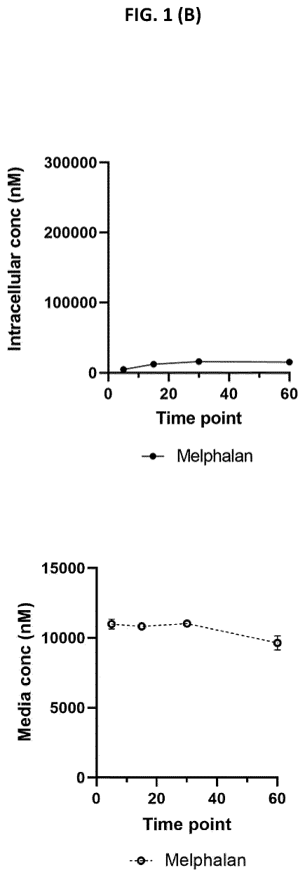

Results: The results are shown in FIG. 1. FIG. 1(A) shows the intracellular and extracellular concentrations of bendamustine and the metabolite compound formed from Example compound 1 (Example compound 18). It is seen that Example compound 18 is rapidly formed inside the cells after treatment with Example compound 1 and low levels of it are found in the media outside the cells. These results indicate that the hydrolysis of Example compound 1 took place inside the cells and that the metabolite of Example compound 1 was retained in the cells. Low intracellular concentrations could be detected of bendamustine and the level decreases over time. The extracellular concentration of bendamustine was quite high and decreased overtime.

In FIG. 1(B), there are shown the intracellular and extracellular concentrations of melphalan over the time course of the experiment. Following in vitro treatment of MM.1S cells with melphalan, a low intracellular concentration of melphalan was detected. The extracellular concentration of melphalan was high and constant throughout the experiment.

### Biological Example 4—In Vivo Assessment of Test Compound Cytotoxicity in a Chicken Embryo Xenograft Model

Methods: Inoculation of Fertilized White Leghorn eggs was performed with 3×10⁶ SU-DHL-4 tumour cells on day 9 (post-fertilisation). Viable eggs were injected with 100 μL of either Example compound 1 (8.4 uM (0.008 mg/kg), 33.5 uM (0.033 mg/kg), 167.5 uM (0.164 mg/kg)), bendamustine (8.4 uM (0.005 mg/kg), 33.5 uM (0.02 mg/kg), 167.5 uM (0.1 mg/kg)) or vehicle on day 11, 13, 15 and 17. On day 18, tumours were removed and weighed.

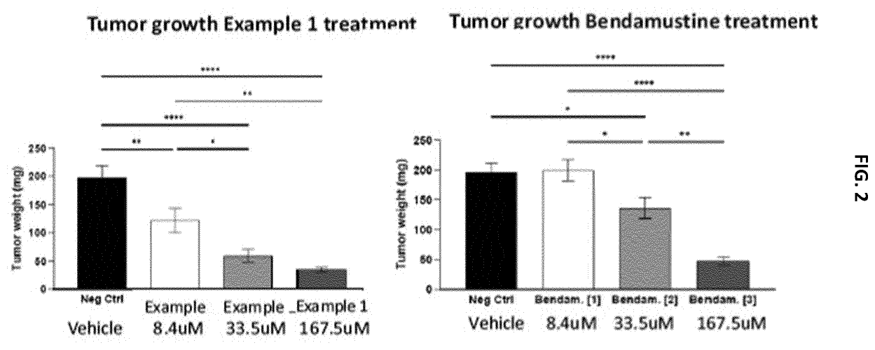

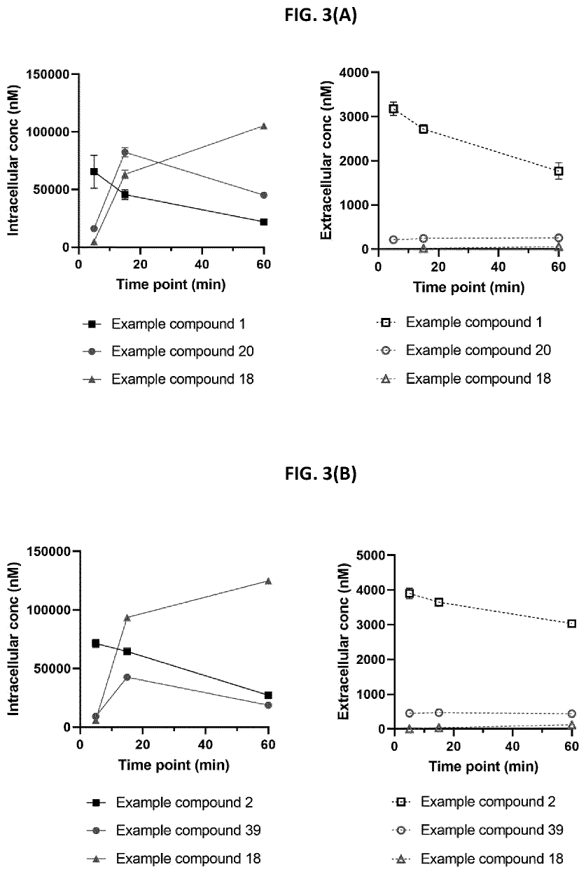

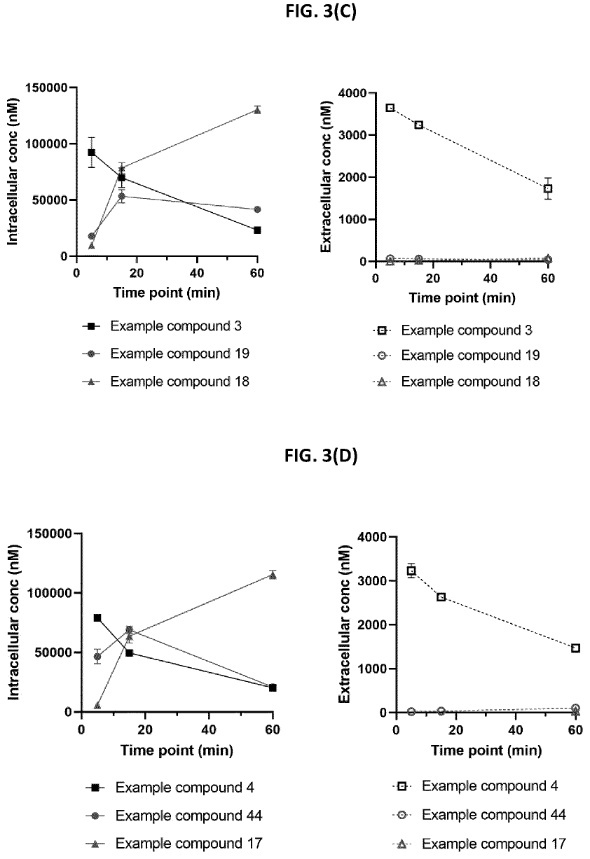

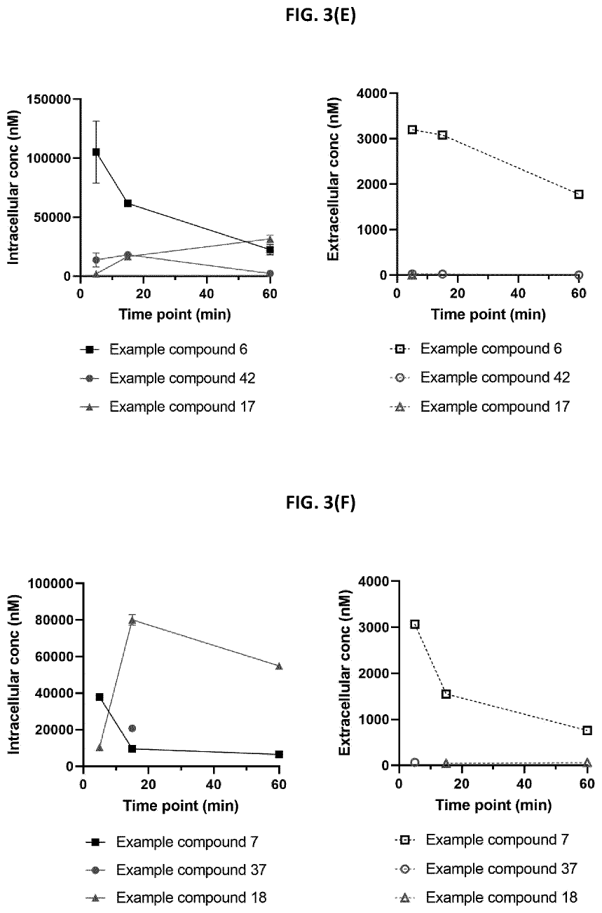

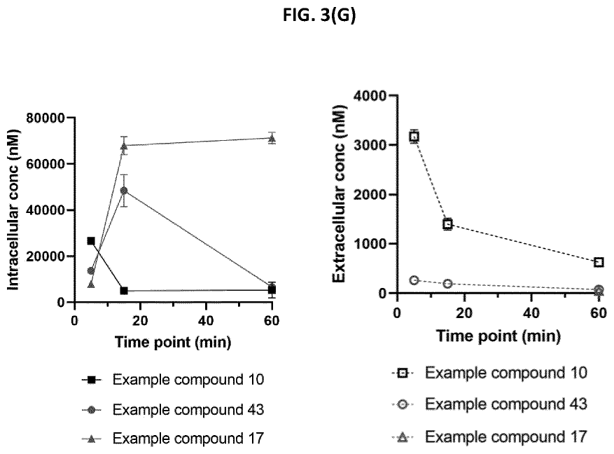

Results: A dose dependent effect on tumour weight was apparent for Example compound 1 (FIG. 2). For Example compound 1, tumour reduction compared to vehicle was 38%, 70% and 82% at 8.4 μM, 33.5 μM and 167.5 μM, respectively. For bendamustine, tumour reduction compared to vehicle was only apparent at 33.5 μM and 167.5 μM, at 30% and 76% reduction respectively. Thus, Example compound 1 shows potent tumour cytotoxicity in this model.

### Biological Example 5—Assessment of Cellular Retention/Efflux of PDC Compounds and Metabolites

Method: Method described in Biological example 3.

Example compounds 1, 2, 3, 4, 6, 7 and 10 were investigated. Intracellular metabolites are formed from all compounds tested. The compound used for cell treatment was metabolised to the ester-hydrolysed compound (here called Metabolite A) and to the amide-hydrolysed compound (called Metabolite B). The example compound structures and the structures of the two metabolites for the respective compounds were as shown in Table 4. The Metabolites B formed under the conditions tested were compounds with structure as Examples 17 and 18:

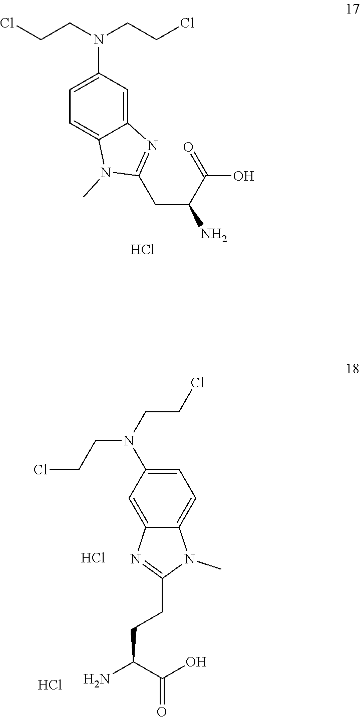 [SMILES: Cn1c(C[C@H](N)C(=O)O)nc2cc(N(CCCl)CCCl)ccc21 ; Cl ; Cl ; Cn1c(CC[C@H](N)C(=O)O)nc2cc(N(CCCl)CCCl)ccc21 ; Cl]

TABLE 4

| Ex | Structure | Metabolite A | Metabolite B |
|---|---|---|---|
| 1 | 1 | 20 | 18 |
| | 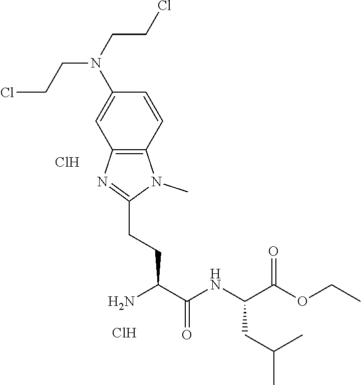 [SMILES: CCOC(=O)[C@H](CC(C)C)NC(=O)[C@@H](N)CCc1nc2cc(N(CCCl)CCCl)ccc2n1C ; Cl ; Cl] | 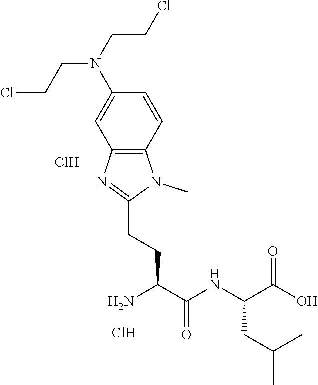 [SMILES: CC(C)C[C@H](NC(=O)[C@@H](N)CCc1nc2cc(N(CCCl)CCCl)ccc2n1C)C(=O)O ; Cl ; Cl] | |
| 2 | 2 | 39 | 18 |
| | 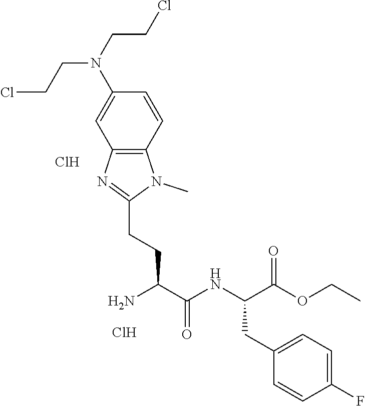 [SMILES: CCOC(=O)[C@H](Cc1ccc(F)cc1)NC(=O)[C@@H](N)CCc1nc2cc(N(CCCl)CCCl)ccc2n1C ; Cl ; Cl] | 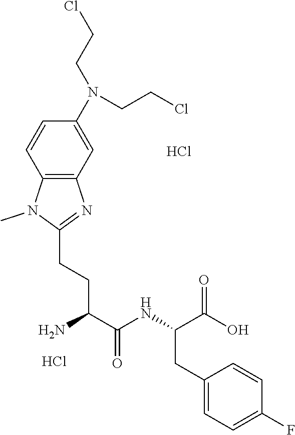 [SMILES: Cn1c(CC[C@H](N)C(=O)N[C@@H](Cc2ccc(F)cc2)C(=O)O)nc2cc(N(CCCl)CCCl)ccc21 ; Cl ; Cl] | |
| 3 | 3 | 19 | 18 |
| | 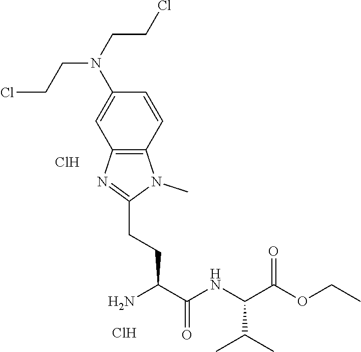 [SMILES: CCOC(=O)[C@@H](NC(=O)[C@@H](N)CCc1nc2cc(N(CCCl)CCCl)ccc2n1C)C(C)C ; Cl ; Cl] | 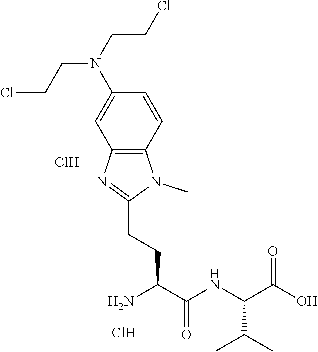 [SMILES: CC(C)[C@H](NC(=O)[C@@H](N)CCc1nc2cc(N(CCCl)CCCl)ccc2n1C)C(=O)O ; Cl ; Cl] | |
| 4 | 4 | 44 | 17 |
| | 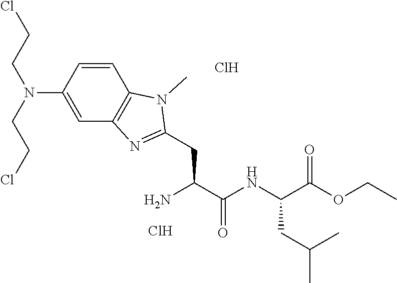 [SMILES: CCOC(=O)[C@H](CC(C)C)NC(=O)[C@@H](N)Cc1nc2cc(N(CCCl)CCCl)ccc2n1C ; Cl ; Cl] | 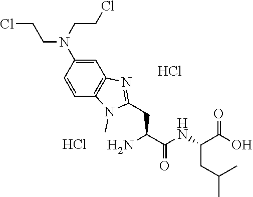 [SMILES: CC(C)C[C@H](NC(=O)[C@@H](N)Cc1nc2cc(N(CCCl)CCCl)ccc2n1C)C(=O)O ; Cl ; Cl] | |
| 6 | 6 | 42 | 17 |
| | 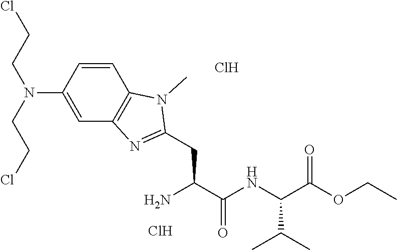 [SMILES: CCOC(=O)[C@@H](NC(=O)[C@@H](N)Cc1nc2cc(N(CCCl)CCCl)ccc2n1C)C(C)C ; Cl ; Cl] | 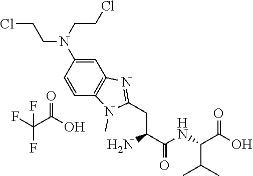 | |
| 7 | 7 | 37 | 18 |
| | 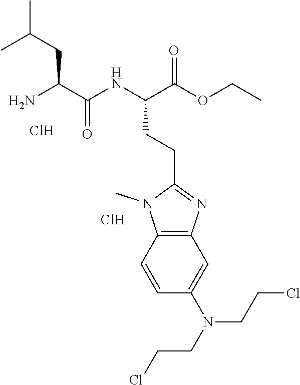 [SMILES: CCOC(=O)[C@H](CCc1nc2cc(N(CCCl)CCCl)ccc2n1C)NC(=O)[C@@H](N)CC(C)C ; Cl ; Cl] | 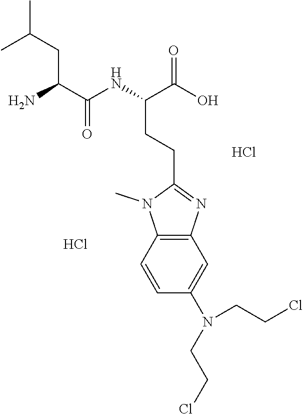 [SMILES: CC(C)C[C@H](N)C(=O)N[C@@H](CCc1nc2cc(N(CCCl)CCCl)ccc2n1C)C(=O)O ; Cl ; Cl] | |
| 10 | 10 | 43 | 17 |
| | 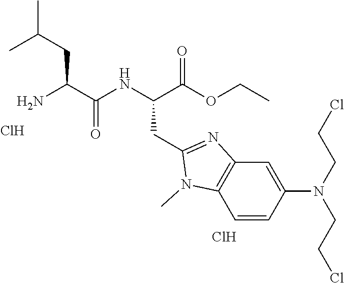 [SMILES: Cl ; CCOC(=O)[C@H](Cc1nc2cc(N(CCCl)CCCl)ccc2n1C)NC(=O)[C@@H](N)CC(C)C ; Cl] | 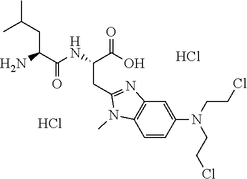 [SMILES: CC(C)C[C@H](N)C(=O)N[C@@H](Cc1nc2cc(N(CCCl)CCCl)ccc2n1C)C(=O)O ; Cl ; Cl] | |

The intracellular concentration of the metabolites slowly decreased over time. Little or undetectable extracellular concentrations of the metabolites can be detected in the media outside cells. These results indicate that hydrolysis of the Example compounds took place inside the cells and that the metabolites of the Example compounds were retained in the cells.

### Biological Example 6—Assessment of Direct DNA Damage by Test Compounds

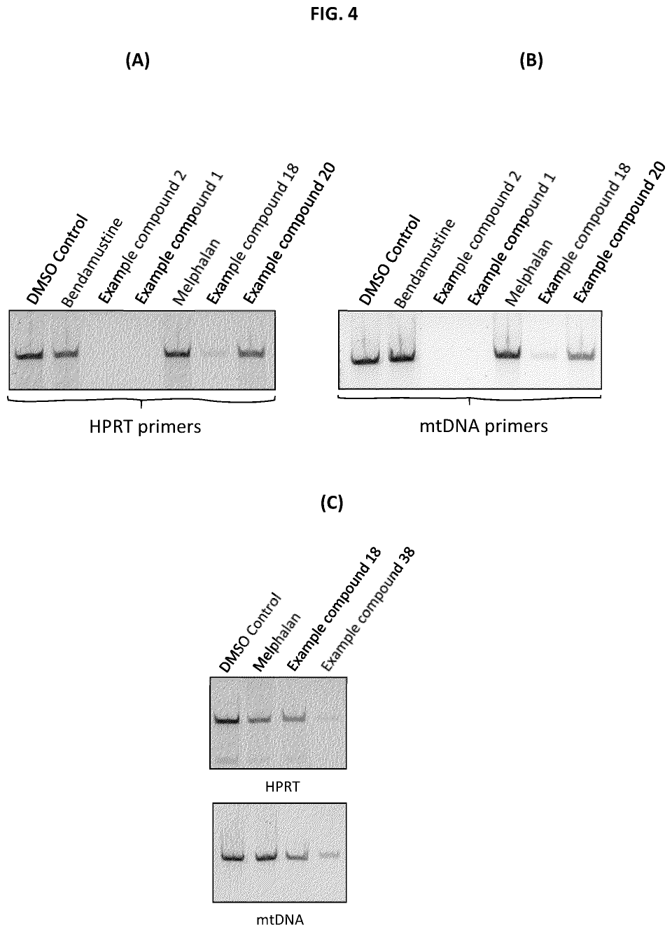

Method: DNA was prepared from MM.1S cells by QIAGEN genomic tip 20/G according to protocol described in Furda et al., Methods Mol Biol, 2012. 1 μg of DNA was treated with 0.25 μM compound for 30 minutes at 37° C. DMSO was used as control treatment. 20 ng of the treated DNA was PCR amplified using Phusion Hot Start II High Fidelity PCR Master Mix (Thermo Scientific) and primers directed to either HPRT or primers directed to mitochondrial DNA (mtDNA) was used, generating 10.4 or 8.9 kb fragments, respectively. The PCR reactions were separated on a 1% agarose gel and visualized by ChemiDoc™ MP Imaging System (BioRad). Example compounds 1, 2 and 38 were investigated, as were compounds 18 and 20, which are metabolites of compound 1. They were compared with bendamustine, melphalan and DMSO control. The results for compounds 1, 2, 18 and 20 with the HPRT primers are shown in FIG. 4(A); the results with the mtDNA primers are shown in FIG. 4(B). The results for compounds 18 and 38 with the HPRT primers and the mtDNA primers are shown in FIG. 4(C).

Results: It is seen in FIG. 4 that Melphalan and Bendamustine had no or very minor effect on DNA damage on both nuclear and mitochondrial DNA at the concentration tested. Example compounds 1 and 2 had a strong DNA damaging effect on both nuclear and mitochondrial DNA, as shown by the fact that no PCR bands could be detected on the gel. Example compounds 18 and 38 effectively damaged both nuclear and mitochondrial DNA and only a weak PCR band could be detected. Example compound 20 also had a DNA damaging effect but to a lesser degree compared to Example compounds 1, 2 and 18, but greater than Bendamustine or Melphalan.

### Biological Example 7—Assessment of DNA Fragmentation Caused by Test Compounds

Method: 1×10⁶ MM.1S cells (CRL-2974, ATCC) were treated with Example compound 1 or Bendamustine at 0.006, 0.06, 0.6 or 6 μM for 24 h. After incubation, the cells were washed with PBS (10010-015, Gibco), fixed in Flow Cytometry Fixation Buffer (FC004, R&D Systems) for 10 min and stored in 70% ethanol at −20° C. overnight. The deoxythymidine analog (BrdUTP) was present in the experiment and served to label the DNA break sites. Next day, the cells were washed and stained using an APO-BrdU™ TUNEL Assay Kit, according to the manufacturer's protocol (A23210, Invitrogen), followed by the analysis using BD FACSCanto™ II. The percentage of TUNEL positive cells indicates the percentage of cells with fragmented DNA.

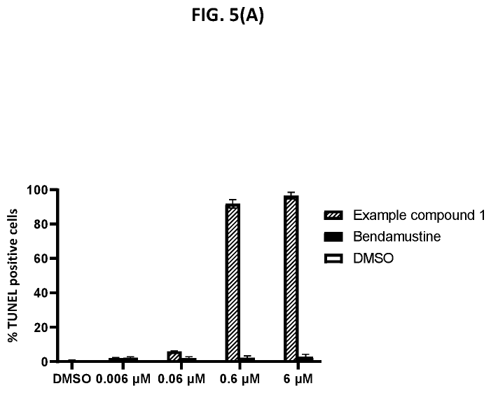

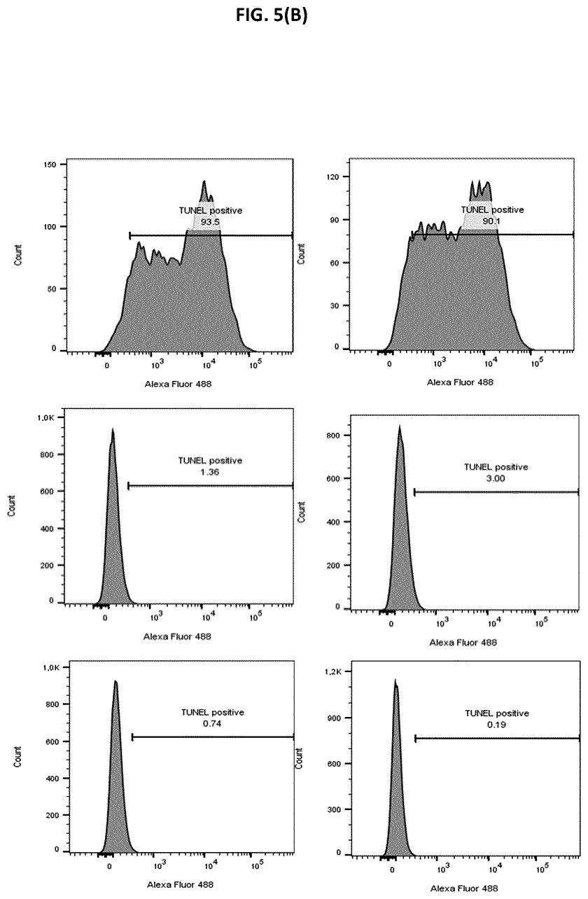

Results: The dose response effect of Example compound 1 and Bendamustine on DNA damage at 24-h incubation is shown in FIG. 5(A). All data were expressed as mean with SD (n=2). 24-h exposure to Example compound 1 induced DNA damage in a dose-dependent manner, while no obvious effect of Bendamustine on DNA damage was found upon 24-h incubation. Representative FACS histograms of Alexa fluor 488 intensity after 24-h treatment of 0.6 μM test compound or Bendamustine (n=2) are shown in FIG. 5(B). The top pair of FACS histograms are for Example compound 1; the middle pair are for Bendamustine and the bottom pair are for the DMSO control. Noticeably, the treatment with 0.6 μM Example compound 1 for 24 h caused DNA breaks in >90% of tested cells, but that was not the case for Bendamustine. Example compound 1 is more potent in causing DNA damage.

1. A compound according to formula (I), or a pharmaceutically acceptable salt, ester, amide or carbamate thereof, including a salt of such an ester, amide or carbamate 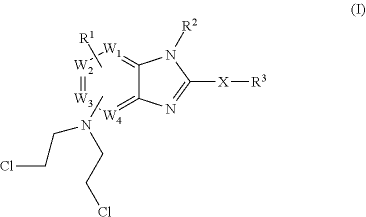
wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is a group according to formula (11): 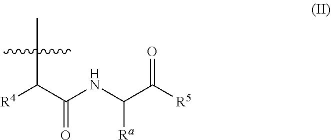
or formula (III): 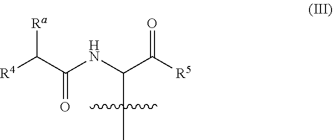
wherein,
R⁴ is selected from the group consisting of N(R^{c})(R^{d}) and formula (IV): 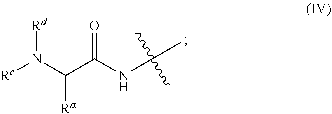
and
when R⁴ is formula (IV), R⁵ is R⁵; and when R⁴ is N(R^{c})(R^{d}), R⁵ is selected from the group consisting of R^{b} and formula (V): 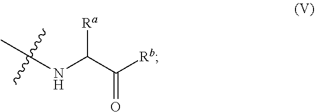
wherein each R¹ is independently selected from the group consisting of H;
C₁₋₆alkyl; —CH₂-phenyl; or —CH₂-3 to 12-membered heterocyclyl comprising 1, 2, 3 or 4 heteroatoms selected from N, O and S; wherein said C₁₋₆alkyl is optionally substituted by 1, 2 or 3 substituents independently selected from the group consisting of —OH, —OC₁₋₆alkyl, —NH₂, —NHC(═NH)NH₂, —C(O)OH, —C(O)NH₂, —SH, —SCH₃, and halogen; and said phenyl or heterocyclyl is optionally substituted by 1, 2 or 3 substituents independently selected from the group consisting of halogen, —NH₂, —OH, —OC₁₋₆alkyl and —NO₂;
R^{b} is selected from the group consisting of —OH; —N(R^{e})(R^{f}); and —OC₁₋₆alkyl optionally substituted by one or more groups selected from halogen, —OH, —CN, —N(R^{e})(R^{f}), —C₆₋₁₀aryl, or 3 to 12 membered heterocycle comprising one or more O, N or S atoms and optionally substituted by 1, 2 or 3 halogens, and/or wherein said alkyl is optionally interrupted by 1, 2 or 3 O, N or S atoms;
R^{c} and R^{d} are each independently selected from the group consisting of H; —C₁₋₆alkyl, C(O)C₁₋₆alkyl; and —CH₂-phenyl, wherein said alkyl or said phenyl is optionally substituted by 1, 2 or 3 groups selected from halogen; and
R^{e} and R^{f} are each independently selected from the group consisting of H and —C₁₋₆alkyl, wherein said alkyl is optionally substituted by 1, 2 or 3 groups selected from halogen; or R^{e} and R^{f} together with the nitrogen atom to which they are attached form a 4-, 5- or 6-membered heterocycle which is optionally substituted by 1, 2 or 3 groups selected from halogen.

2. A compound as claimed in claim 1 which has the structure (Ia): 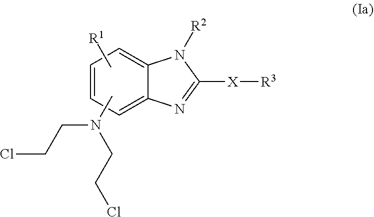
for example structure (Ib):

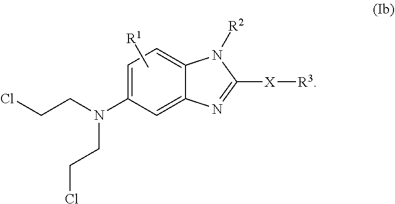

3. A compound as claimed in claim 1 or claim 2 wherein R¹ is H.

4. A compound as claimed in any one of claims 1 to 3 wherein R² is selected from the group consisting of phenyl optionally substituted by 1, 2 or 3 halogens; and —C₁₋₆alkyl optionally substituted by 1, 2 or 3 halogens; for example —C₁₋₄alkyl optionally substituted by 1, 2 or 3 halogens, for example methyl.

5. A compound as claimed in any one of claims 1 to 4 wherein X is —CH₂— or —CH₂—CH₂—.

6. A compound as claimed in any one of claims 1 to 5 wherein R³ is a group according to formula (IIa): 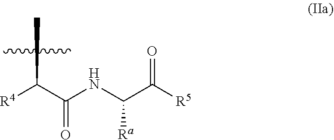
or formula (IIIa):

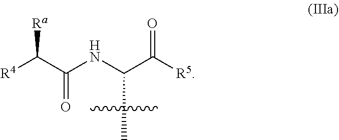

7. A compound as claimed in any one of claims 1 to 6 wherein R⁴ is selected from the group consisting of N(R^{c})(R^{d}) and formula (IVa):

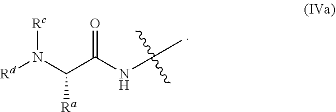

8. A compound as claimed in any one of claims 1 to 7 wherein, when R⁴ is formula (IV) or (IVa), R⁵ is R^{b}; and when R⁴ is N(R^{c})(R^{d}), R⁵ is selected from the group consisting of R^{b} and formula (Va), wherein formula (Va) has the following structure,

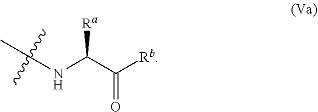

9. A compound as claimed in any one of claims 1 to 8 wherein R⁴ is NH₂.

10. A compound as claimed in any one of claims 1 to 9 wherein each R^{a} is independently selected from the group consisting of C₁₋₆alkyl and —CH₂-phenyl; wherein said C₁₋₆alkyl is optionally substituted by —OH; —NH₂; —NHC(═NH)NH₂; —C(O)OH; —C(O)NH₂; —SH; —SCH₃; or halogen (for example, F or Cl); and said phenyl is optionally substituted by 1, 2 or 3 substituents independently selected from the group consisting of halogen (for example, F or Cl); —NH₂; —OH; —O—C₁₋₆alkyl; and —NO₂.

11. A compound as claimed in claim 10 wherein each R^{a} is selected from the group consisting of —C₁₋₆alkyl and —CH₂-phenyl; wherein said phenyl is optionally substituted by 1, 2 or 3 halogen substituents.

12. A compound as claimed in any one of claims 1 to 11 wherein when R⁴ is formula (IV) or (IVa), R⁵ is —OC₁₋₆alkyl; and when R⁴ is NH₂, R⁵ is —OC₁₋₆alkyl or formula (V) or (Va).

13. A compound as claimed in claim 12 wherein when R^{b} is —OC₁₋₆alkyl, it is selected from the group consisting of methoxy, ethoxy and isopropoxy.

14. A compound as claimed in claim 1, wherein the compound is selected from the group consisting of:
Ethyl (2S)-2-[[(2S)-2-amino-4-[5-[bis(2-chloroethyl)amino]-1-methyl-benzimidazol-2-yl]butanoyl]amino]-4-methyl-pentanoate (Example compound 1);
Ethyl (2S)-2-[[(2S)-2-amino-4-[5-[bis(2-chloroethyl)amino]-1-methyl-benzimidazol-2-yl]butanoyl]amino]-3-(4-fluorophenyl)propanoate (Example compound 2);
Ethyl (2S)-2-[[(2S)-2-amino-4-[5-[bis(2-chloroethyl)amino]-1-methyl-benzimidazol-2-yl]butanoyl]amino]-3-methyl-butanoate (Example compound 3);
Ethyl (2S)-2-[[(2S)-2-amino-3-[5-[bis(2-chloroethyl)amino]-1-methyl-benzimidazol-2-yl]propanoyl]amino]-4-methyl-pentanoate (Example compound 4);
Ethyl (2S)-2-[[(2S)-2-amino-3-[5-[bis(2-chloroethyl)amino]-1-methyl-benzimidazol-2-yl]propanoyl]amino]-3-(4-fluorophenyl)propanoate (Example compound 5);
Ethyl (2S)-2-[[(2S)-2-amino-3-[5-[bis(2-chloroethyl)amino]-1-methyl-benzimidazol-2-yl]propanoyl]amino]-3-methyl-butanoate (Example compound 6);
Ethyl (2S)-2-[[(2S)-2-amino-4-methyl-pentanoyl]amino]-4-[5-[bis(2-chloroethyl)amino]-1-methyl-benzimidazol-2-yl]butanoate (Example compound 7);
Ethyl (2S)-2-[[(2S)-2-amino-3-(4-fluorophenyl)propanoyl]amino]-4-[5-[bis(2-chloroethyl)amino]-1-methyl-benzimidazol-2-yl]butanoate (Example compound 8);
Ethyl (2S)-2-[[(2S)-2-amino-3-methyl-butanoyl]amino]-4-[5-[bis(2-chloroethyl)amino]-1-methyl-benzimidazol-2-yl]butanoate (Example compound 9);
Ethyl (2S)-2-[[(2S)-2-amino-4-methyl-pentanoyl]amino]-3-[5-[bis(2-chloroethyl)amino]-1-methyl-benzimidazol-2-yl]propanoate (Example compound 10);
Ethyl (2S)-2-[[(2S)-2-amino-3-(4-fluorophenyl)propanoyl]amino]-3-[5-[bis(2-chloroethyl)amino]-1-methyl-benzimidazol-2-yl]propanoate (Example compound 11);
Ethyl (2S)-2-[[(2S)-2-amino-3-methyl-butanoyl]amino]-3-[5-[bis(2-chloroethyl)amino]-1-methyl-benzimidazol-2-yl]propanoate (Example compound 12);
Ethyl (2S)-2-[[(2S)-2-amino-4-[5-[bis(2-chloroethyl)amino]-1-phenyl-benzimidazol-2-yl]butanoyl]amino]-4-methyl-pentanoate (Example compound 13);
Methyl (2S)-2-[[(2S)-2-amino-3-[5-[bis(2-chloroethyl)amino]-1-methyl-benzimidazol-2-yl]propanoyl]amino]-4-methyl-pentanoate (Example compound 14);
Isopropyl (2S)-2-[[(2S)-2-amino-3-[5-[bis(2-chloroethyl)amino]-1-methyl-benzimidazol-2-yl]propanoyl]amino]-4-methyl-pentanoate (Example compound 15); ethyl (2S)-2-[[(2S)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 16);
Ethyl (2S)-2-[[(2R)-2-[[(2S)-2-amino-3-(4-fluorophenyl)propanoyl]amino]-4-methyl-pentanoyl]amino]-4-[5-[bis(2-chloroethyl)amino]-1-methyl-benzimidazol-2-yl]butanoate (Example compound 21)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-3-(4-fluorophenyl)propanoate (Example compound 22)
Ethyl (2S)-2-[[(2S)-2-[[(2S)-2-amino-4-[5-[bis(2-chloroethyl)amino]-1-methyl-benzimidazol-2-yl]butanoyl]amino]-4-methyl-pentanoyl]amino]-4-methyl-pentanoate (Example compound 23)
2-Morpholinoethyl (2S)-2-[[(2S)-2-amino-4-[5-[bis(2-chloroethyl)amino]-1-methyl-benzimidazol-2-yl]butanoyl]amino]-4-methyl-pentanoate (Example compound 24) 2-Isopropoxyethyl (2S)-2-[[(2S)-2-amino-4-[5-[bis(2-chloroethyl)amino]-1-methyl-benzimidazol-2-yl]butanoyl]amino]-4-methyl-pentanoate (Example compound 25) Isopropyl (2S)-2-[[(2S)-2-amino-4-[5-[bis(2-chloroethyl)amino]-1-methyl-benzimidazol-2-yl]butanoyl]amino]-4-methyl-pentanoate (Example compound 26)
Methyl (2S)-2-[[(2S)-2-amino-4-[5-[bis(2-chloroethyl)amino]-1-methyl-benzimidazol-2-yl]butanoyl]amino]-1-methyl-pentanoate (Example compound 27)
3-(Dimethylamino)propyl (2S)-2-[[(2S)-2-amino-4-[5-[bis(2-chloroethyl)amino]-1-methyl-benzimidazol-2-yl]butanoyl]amino]-4-methyl-pentanoate (Example compound 28)
(2-Methoxy-1-methyl-ethyl) (2S)-2-[[(2S)-2-amino-4-[5-[bis(2-chloroethyl)amino]-1-methyl-benzimidazol-2-yl]butanoyl]amino]-4-methyl-pentanoate (Example compound 29)
(2S)-2-[[(2S)-2-Amino-4-[5-[bis(2-chloroethyl)amino]-1-methyl-benzimidazol-2-yl]butanoyl]amino]-N,N,4-trimethyl-pentanamide (Example compound 30)
(2S)-2-[[(2S)-2-amino-4-[5-[bis(2-chloroethyl)amino]-1-methyl-benzimidazol-2-yl]butanoyl]amino]-N,4-dimethyl-pentanamide (Example compound 31)
Ethyl (2S)-2-[[(2S)-4-[5-[bis(2-chloroethyl)amino]-1-methyl-benzimidazol-2-yl]-2-(methylamino)butanoyl]amino]-4methyl-pentanoate (Example compound 32)
Ethyl (2S)-2-[[(2S)-2-acetamido-4-[5-[bis(2-chloroethyl)amino]-1-methyl-benzimidazol-2-yl]butanoyl]amino]-3-(4-fluorophenyl)propanoate (Example compound 34)
Ethyl (2S)-2-[[(2S)-2-acetamido-3-[5-[bis(2-chloroethyl)amino]-1-methyl-benzimidazol-2-yl]propanoyl]amino]-4-ethyl-pentanoate (Example compound 35)
Ethyl (2S)-2-[[(2S)-2-acetamido-3-[5-[bis(2-chloroethyl)amino]-1-ethyl-benzimidazol-2-yl]propanoyl]amino]-3-(4-fluorophenyl)propanoate (Example compound 36)
Ethyl (2S)-2-amino-4-[5-[bis(2-chloroethyl)amino]-1-methyl-benzimidazol-2-yl]butanoate (Example compound 38)
Ethyl (2R)-2-[[(2S)-2-amino-4-[5-[bis(2-chloroethyl)amino]-1-methyl-benzimidazol-2-yl]butanoyl]amino]-4-methyl-pentanoate (Example compound 45)
Ethyl (2S)-2-[[(2R)-2-amino-4-[5-[bis(2-chloroethyl)amino]-1-methyl-benzimidazol-2-yl]butanoyl]amino]-4-methyl-pentanoate (Example compound 46)
Ethyl (2)-2-[[(2)-2-amino-4-[5-[bis(2-chloroethyl)amino]-1-methyl-benzimidazol-2-yl]butanoyl]amino]-4-methyl-pentanoate (Example compound 47)
Ethyl (2S)-2-[[(2S)-2-amino-4-[6-[bis(2-chloroethyl)amino]-3-methyl-imidazo[4,5-b]pyridin-2-yl]butanoyl]amino]-4-methyl-pentanoate (Example compound 48); and
Ethyl (2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoate (Example compound 50)
or a pharmaceutically acceptable salt, amide or carbamate thereof, including a salt of such a amide or carbamate.

15. A compound as claimed in claim 1, wherein the compound is selected from the group consisting of:
(2S)-2-[[(2S)-2-amino-4-[5-[bis(2-chloroethyl)amino]-1-methyl-benzimidazol-2-yl]butanoyl]amino]-3-methyl-butanoic acid (Example compound 19); and
(2S)-2-[[(2S)-2-amino-4-[5-[bis(2-chloroethyl)amino]-1-methyl-benzimidazol-2-yl]butanoyl]amino]-4-methyl-pentanoic acid (Example compound 20);
(2S)-2-[[(2S)-4-[5-[bis(2-chloroethyl)amino]-1-methyl-benzimidazol-2-yl]-2-(methylamino)butanoyl]amino]-4-methyl-pentanoic acid (Example compound 33)
(2S)-2-[[(2S)-2-Amino-4-methyl-pentanoyl]amino]-4-[5-[bis(2-chloroethyl)amino]-1-methyl-benzimidazol-2-yl]butanoic acid (Example compound 37)
(2S)-2-[[(2S)-2-Amino-4-[5-[bis(2-chloroethyl)amino]-1-methyl-benzimidazol-2-yl]butanoyl]amino]-3-(4-fluorophenyl)propanoic acid (Example compound 39)
(2S)-2-[[(2S)-2-Amino-4-[5-[bis(2-chloroethyl)amino]-1-phenyl-benzimidazol-2-yl]butanoyl]amino]-4-methyl-pentanoic acid (Example compound 40)
2S)-2-[[(2S)-2-amino-3-[5-[bis(2-chloroethyl)amino]-1-methyl-benzimidazol-2-yl]propanoyl]amino]-3-methyl-butanoic acid (Example compound 42)
(2S)-2-[[(2S)-2-amino-4-methyl-pentanoyl]amino]-3-[5-[bis(2-chloroethyl)amino]-1-methyl-benzimidazol-2-yl]propanoic acid (Example compound 43)
(2S)-2-[[(2S)-2-amino-3-[5-[bis(2-chloroethyl)amino]-1-methyl-benzimidazol-2-yl]propanoyl]amino]-4-methyl-pentanoic acid (Example compound 44); and
(2S)-2-[[(2S)-2-amino-4-[5-[bis(2-chloro-1,1,2,2-tetradeutero-ethyl)amino]-1-methyl-benzimidazol-2-yl]butanoyl]amino]-4-methyl-pentanoic acid (Example compound 51)
or a salt thereof.

16. A pharmaceutical composition comprising a compound as defined in any one of claims 1 to 15, together with a pharmaceutically acceptable carrier.

17. A pharmaceutical composition as claimed in claim 16 which further comprises an additional therapeutic agent, for example a steroid, checkpoint inhibitor, nuclear transport inhibitor, anti-apoptotic inhibitor, adoptive cell therapy, bi-specific T-cell engager (BiTE), immunomodulatory imide drug (IMiD), proteasome inhibitor (PI), histone deacetylase (HDAC) inhibitor, peptide drug conjugate (PDC), an alkylator or DNA intercalator.

18. A compound as claimed in any one of claims 1 to 15, or a pharmaceutical composition as claimed in claim 16 or 17, for use as a medicament.

19. A compound as claimed in any one of claims 1 to 15, or a pharmaceutical composition as claimed in claim 16 or 17, for use in the treatment or prophylaxis of cancer.

20. A compound or composition for use as claimed in claim 19 wherein the cancer is selected from multiple myeloma, osteosarcoma, breast cancer, lung cancer, ovarian cancer, leukaemia and lymphoma; for example multiple myeloma, acute myeloid leukemia or lymphoma.

21. A compound or composition for use as claimed in claim 19 or 20, wherein the compound or composition is administered (for example simultaneously, sequentially or separately) with one or more additional therapeutic agent(s), for example a steroid, checkpoint inhibitor, nuclear transport inhibitor, anti-apoptotic inhibitor, adoptive cell therapy, bi-specific T-cell engager (BiTE), immunomodulatory imide drug (IMiD), proteasome inhibitor (PI), histone deacetylase (HDAC) inhibitor, peptide drug conjugate (PDC), an alkylator or DNA intercalator.

22. A method for treating a patient which comprises administering a pharmaceutically effective amount of a compound as claimed in any one of claims 1 to 15 or a pharmaceutical composition as claimed in claim 16 or 17.

23. A method for the treatment or prophylaxis of cancer, comprising administering an effective amount of a compound as claimed in any one of claims 1 to 15 or a pharmaceutical composition as claimed in claim 16 or 17, for example wherein said cancer is any one of multiple myeloma, osteosarcoma, breast cancer, lung cancer, ovarian cancer, leukaemia and lymphoma; for example multiple myeloma, acute myeloid leukemia or lymphoma.

24. The use of a compound as claimed in any one of claims 1 to 15 for the manufacture of a medicament for the treatment or prophylaxis of cancer, for example wherein said cancer is any one of multiple myeloma, osteosarcoma, breast cancer, lung cancer, ovarian cancer, leukaemia and lymphoma; for example multiple myeloma, acute myeloid leukemia or lymphoma.

25. A compound according to formula (I), or a salt or solvate thereof 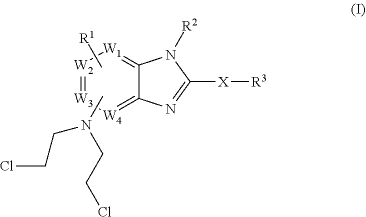
wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl, optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen; and
R³ is formula (VIa): 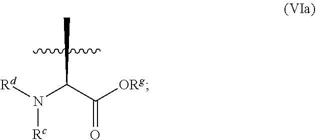
wherein, R^{c} and R^{d} are each independently selected from the group consisting of H, —C₁₋₆alkyl, C(O)C₁₋₆alkyl and —CH₂-phenyl, wherein said alkyl or said phenyl is optionally substituted by 1, 2 or 3 groups selected from halogen; and
R^{g} is selected from the group consisting of H and —C₁₋₆alkyl, wherein said alkyl is optionally substituted by 1, 2 or 3 groups selected from halogen.

26. A compound as claimed in claim 25 wherein the structure is (Ia) or (Ib) as shown in claim 2, or R¹, R² or X are as recited in any of claims 3 to 5; R^{c} and R^{d} are each H; and R^{g} is H.

27. A compound as claimed in claim 25 wherein the compound is selected from the group consisting of:
(2S)-2-amino-3-[5-[bis(2-chloroethyl)amino]-1-methyl-benzimidazol-2-yl]propanoic acid (Example compound 17);
(S)-2-amino-4-(5-(bis(2-chloroethyl)amino)-1-methyl-1H-benzo[d]imidazol-2-yl)butanoic acid (Example compound 18);
Ethyl (2S)-2-amino-4-[5-[bis(2-chloroethyl)amino]-1-methyl-benzimidazol-2-yl]butanoate (Example compound 38)
(2S)-2-Amino-4-[5-[bis(2-chloroethyl)amino]-1-phenyl-benzimidazol-2-yl]butanoic acid (Example compound 41); and
(2S)-2-amino-4-[5-[bis(2-chloro-1,1,2,2-tetradeutero-ethyl)amino]-1-methyl-benzimidazol-2-yl]butanoic acid (Example compound 49);
or a salt thereof.

28. A compound according to formula (I), or a salt or solvate thereof 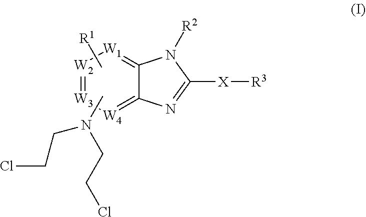
wherein,
X is C₁₋₆alkylene;
W₁, W₂, W₃ and W₄ are each CH, or one of W₁, W₂, W₃ and W₄ is N and the others are CH;
R¹ is selected from the group consisting of H; C₁₋₄alkyl optionally substituted by 1, 2 or 3 groups independently selected from halogen; and halogen;
R² is selected from the group consisting of H; phenyl optionally substituted by 1, 2 or 3 groups independently selected from halogen; and C₁₋₆alkyl optionally substituted by 1, 2 or 3 groups independently selected from halogen;
R³ is formula (VI): 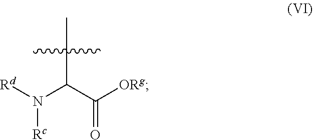
wherein, R^{c} and R^{d} are each independently selected from the group consisting of H, —C₁₋₆alkyl, C(O)C₁₋₆alkyl and —CH₂-phenyl, wherein said alkyl or said phenyl is optionally substituted by 1, 2 or 3 groups selected from halogen; and
R^{g} is selected from the group consisting of H and —C₁₋₆alkyl, wherein said alkyl is optionally substituted by 1, 2 or 3 groups selected from halogen,
for use in the treatment of a cancer selected from the group consisting of multiple myeloma, osteosarcoma, breast cancer, lung cancer, ovarian cancer, leukaemia and lymphoma; for example multiple myeloma, acute myeloid leukemia or lymphoma.

29. A method for the treatment of a cancer selected from the group consisting of multiple myeloma, osteosarcoma, breast cancer, lung cancer, ovarian cancer, leukaemia and lymphoma; for example multiple myeloma, acute myeloid leukemia or lymphoma, comprising administering an effective amount of a compound as recited in claim 28.
